(19) United States
(12) Patent Application Publication
(10) Pub. No.: US 2007/0208005 A1
(43) Pub. Date: Sep. 6, 2007

(54) TETRAHYDROBENZAZEPINES AS ANTAGONISTS AND/OR REVERSE AGONISTS OF THE HISTAMINE H3 RECEPTOR

(76) Inventors: Christopher Allan Parr, Middlesex (GB); Paula Louise Pickering, Essex (GB); Sanjeet Singh Sehmi, Essex (GB); David Matthew Wilson, Essex (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/573,732

(22) PCT Filed: Aug. 12, 2005
(86) PCT No.: PCT/EP05/08841

§ 371 Date: Feb. 15, 2007

(30) Foreign Application Priority Data
Aug. 16, 2004 (GB) 0418267.1

(51) Int. Cl. A61K 31/55; C07D 413/02; C07D 403/02

(52) U.S. Cl. 514/217.01; 540/594

(57) ABSTRACT

The present invention relates to novel benzazepine derivatives having pharmaceutical activity, processes for their preparation, to compositions containing them and to their use in the treatment of neurological and psychiatric disorders.

The present invention relates to novel benzazepine derivatives having pharmacological activity, processes for their preparation, to compositions containing them and to their use in the treatment of neurological and psychiatric disorders.

JP 2001226269 and WO 00/23437 (Takeda Chem Ind Ltd) describe a series of benzazepine derivatives which are claimed to be useful in the treatment of obesity. DE 2207430, U.S. Pat. No. 4,210,749 and FR 2171879 (Pennwalt Corp) and GB 1268243 (Wallace and Tieman Inc) all describe a series of benzazepine derivatives which are claimed as being antagonists for narcotics (such as morphine or codeine) and also anti-histamines and anticholinergic agents. WO 02/14513 (Takeda Chem Ind Ltd) describe a series of benzazepine derivatives with GPR12 activity which are claimed to be useful in the treatment of attention deficit disorder, narcolepsy or anxiety. WO 02/02530 (Takeda Chem Ind Ltd) describe a series of benzazepine derivatives as GPR14 antagonists which are claimed to be useful in the treatment of hypertension, atherosclerosis and cardiac infarction. WO 01/03680 (Isis Innovation Ltd) describe a series of benzazepine derivatives which are claimed as effective agents in the preparation of cells for transplantation in addition to the inhibition of diseases such as diabetes. WO 00/21951 (SmithKline Beecham plc) discloses a series of tetrahydrobenzazepine derivatives as modulators of dopamine D3 receptors which are claimed to be useful as antipsychotic agents. WO 01/87834 (Takeda Chem Ind Ltd) describe a series of benzazepine derivatives as MCH antagonists which are claimed to be useful in the treatment of obesity. WO 02/15934 (Takeda Chem Ind Ltd) describe a series of benzazepine derivatives as urotensin II receptor antagonists which are claimed to be useful in the treatment of neurodegenerative disorders. WO 04/018432 (Eli Lilly and Company) describe a series of substituted azepines as histamine H3 receptor antagonists. WO2004/056369 (Glaxo Group Limited) describe a series of benzazepine derivatives as histamine H3 antagonists for the treatment of neurological and psychiatric disorders. WO 2004/05639 (Glaxo Group Ltd.) describes a series of benzazepine derivatives and their use in the treatment of neurological disorders. U.S. Pat. No. 5,932,590 and WO99/28314 (Merck & Co. Inc.) disclose the use of 1,2,3,4-tetrahydroisoquinolines and homologous compounds as farnesyl-protein transferase inhibitors which are claimed to be useful in the treatment of cancer and other diseases.

The histamine H3 receptor is predominantly expressed in the mammalian central nervous system (CNS), with minimal expression in peripheral tissues except on some sympathetic nerves (Leurs et al., (1998), Trends Pharmacol. Sci. 19, 177-183). Activation of H3 receptors by selective agonists or histamine results in the inhibition of neurotransmitter release from a variety of different nerve populations, including histaminergic and cholinergic neurons (Schlicker et al., (1994), Fundam. Clin. Pharmacol. 8, 128-137). Additionally, in vitro and in vivo studies have shown that H3 antagonists can facilitate neurotransmitter release in brain areas such as the cerebral cortex and hippocampus, relevant to cognition (Onodera et al., (1998), In: The Histamine H3 receptor, ed Leurs and Timmerman, pp 255-267, Elsevier Science B.V.). Moreover, a number of reports in the literature have demonstrated the cognitive enhancing properties of H3 antagonists (e.g. thioperamide, clobenpropit, ciproxifan and GT-2331) in rodent models including the five choice task, object recognition, elevated plus maze, acquisition of novel task and passive avoidance (Giovanni et al., (1999), Behav. Brain Res. 104, 147-155). These data suggest that novel H3 antagonists and/or inverse agonists such as the current series could be useful for the treatment of cognitive impairments in neurological diseases such as Alzheimer's disease and related neurodegenerative disorders.

The present invention provides, in a first aspect, a compound of formula (I) or a pharmaceutically acceptable salt thereof: 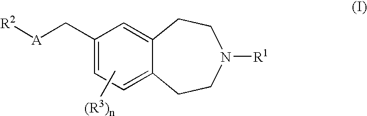
wherein:
R¹ represents C₁₋₆ alkyl or —C₃₋₇ cycloalkyl, wherein the C₃₋₇ cycloalkyl group may optionally be substituted by C₁₋₃ alkyl;
A represents a bond, O, S or NR⁷;
R⁷ represents hydrogen, C₁₋₆ alkyl or aryl;
R² represents -aryl, -heteroaryl, —C₃₋₈ cycloalkyl-Y—C₃₋₈ cycloalkyl, —C₃₋₈ cycloalkyl-Y-aryl, —C₃₋₈ cycloalkyl-Y-heteroaryl, —C₃₋₈ cycloalkyl-Y-heterocyclyl, -aryl-Y—C₃₋₈ cycloalkyl, -aryl-Y-aryl, -aryl-Y-heteroaryl, -aryl-Y-heterocyclyl, -heteroaryl-Y—C₃₋₈ cycloalkyl, -heteroaryl-Y-aryl, -heteroaryl-Y-heteroaryl, -heteroaryl-Y-heterocyclyl, -heterocyclyl-Y—C₃₋₈ cycloalkyl, -heterocyclyl-Y-aryl, -heterocyclyl-Y-heteroaryl or -heterocyclyl-Y-heterocyclyl, such that R² is linked to A via a carbon atom;
Y represents a bond, C₁₋₆ alkyl, CO, CONH, COC₂₋₆ alkenyl, O, SO₂ or NHCOC₁₋₆ alkyl;
R³ represents halogen, C₁₋₆ alkyl, C₁₋₆ alkoxy, cyano, amino or trifluoromethyl;
n is 0, 1 or 2;
wherein said alkyl, cycloalkyl, aryl, heteroaryl and heterocyclyl groups of R² may be optionally substituted by one or more substituents (e.g. 1, 2 or 3) which may be the same or different, and which are selected from the group consisting of halogen, hydroxy, cyano, nitro, ═O, SO₂H, —R⁴, —CO₂R⁴, —COR⁴, —NR⁵R⁶, —C₁₋₆ alkyl-NR⁵R⁶, —C₃₋₈ cycloalkyl-NR⁵R⁶, —CONR⁵R⁶, —NR⁵COR⁶, —NR⁵SO₂R⁶, —OCONR⁵R⁶, —NR⁵CO₂R⁶, —NR⁴CONR⁵R⁶ or —SO₂NR⁵R⁶—SHR⁸, -alkyl-OR⁸, —SOR⁸, —OR⁹, —SO₂R⁹, —OSO₂R⁹, -alkyl-SO₂R⁹, -alkyl-CONHR⁹, -alkyl-SONHR⁹, -alkyl-COR¹⁰, —CO-alkyl-R¹⁰, —O-alkyl-R¹¹ (wherein R⁴, R⁵ and R⁶ independently represent hydrogen, C₁₋₆ alkyl, —C₃₋₈ cycloalkyl, —C₁₋₆ alkyl-C₃₋₈ cycloalkyl, aryl, heterocyclyl or heteroaryl, wherein R⁸ represents C₁₋₆ alkyl, wherein R⁹ represents C₁₋₆ alkyl or aryl, wherein R¹⁰ represents aryl, wherein R¹¹ represents C₃₋₈ cycloalkyl or aryl, and
wherein —NR⁵R⁶ may represent a nitrogen containing heterocyclyl group);
wherein said R⁴, R⁵′, R⁶ R⁸, R⁹, R¹⁰ and R¹¹ groups may be optionally substituted by one or more substituents (e.g. 1, 2 or 3) which may be the same or different, and which are selected from the group consisting of halogen, hydroxy, C₁₋₆ alkyl, C₁₋₆ alkoxy, cyano, amino, ═O or trifluoromethyl;
or solvates thereof.

In one aspect:

R¹ represents —C₃₋₇ cycloalkyl optionally substituted by C₁₋₃ alkyl;

A represents a bond, O, S or NR⁷;

R⁷ represents hydrogen, C₁₋₆ alkyl or aryl;

R² represents -aryl, -heteroaryl, —C₃₋₈ cycloalkyl-Y—C₃₋₈ cycloalkyl, —C₃₋₈ cycloalkyl-Y-aryl, —C₃₋₈ cycloalkyl-Y-heteroaryl, —C₃₋₈ cycloalkyl-Y-heterocyclyl, -aryl-Y—C₃₋₈ cycloalkyl, -aryl-Y-aryl, -aryl-Y-heteroaryl, -aryl-Y-heterocyclyl, -heteroaryl-Y—C₃₋₈ cycloalkyl, -heteroaryl-Y-aryl, -heteroaryl-Y-heteroaryl, -heteroaryl-Y-heterocyclyl, -heterocyclyl-Y—C₃₋₈ cycloalkyl, -heterocyclyl-Y-aryl, -heterocyclyl-Y-heteroaryl or -heterocyclyl-Y-heterocyclyl, such that R² is linked to A via a carbon atom;

Y represents a bond, C₁₋₆ alkyl, CO, CONH, COC₂₋₆ alkenyl, O, SO₂ or NHCOC₁₋₆ alkyl;

R³ represents halogen, C₁₋₆ alkyl, C₁₋₆ alkoxy, cyano, amino or trifluoromethyl;

n is 0, 1 or 2;

wherein said alkyl, cycloalkyl, aryl, heteroaryl and heterocyclyl groups of R² may be optionally substituted by one or more substituents (e.g. 1, 2 or 3) which may be the same or different, and which are selected from the group consisting of halogen, hydroxy, cyano, nitro, ═O, C₁₋₆ alkyl, haloC₁₋₆ alkyl, haloC₁₋₆ alkoxy, C₁₋₆ alkoxy, arylC₁₋₆ alkoxy, C₁₋₆ alkylthio, C₁₋₆ alkoxyC₁₋₆ alkyl, C₃₋₇ cycloalkylC₁₋₆ alkoxy, C₁₋₆ alkanoyl, C₁₋₆ alkoxycarbonyl, C₁₋₆ alkylsulfonyl, C₁₋₆ alkylsulfinyl, C₁₋₆ alkylsulfonyloxy, C₁₋₆ alkylsulfonylC₁₋₆ alkyl, sulfonyl, arylsulfonyl, arylsulfonyloxy, arylsulfonylC₁₋₆ alkyl, aryloxy, C₁₋₆ alkylsulfonamido, C₁₋₆ alkylamino, C₁₋₆ alkylamido, —R⁴, —CO₂R⁴, —COR⁴, C₁₋₆ alkylsulfonamidoC₁₋₆ alkyl, C₁₋₆ alkylamidoC₁₋₆ alkyl, arylsulfonamido, arylcarboxamido, arylsulfonamidoC₁₋₆ alkyl, arylcarboxamidoC₁₋₆ alkyl, aroyl, aroylC₁₋₆ alkyl, arylC₁₋₆ alkanoyl, or a group —NR⁵R⁶, —C₁₋₆ alkyl-NR⁵R⁶, —C₃₋₈ cycloalkyl-NR⁵R⁶, —CONR⁵R⁶, —NR⁵COR⁶, —NR⁵SO₂R⁶, —OCONR⁵R⁶, —NR⁵CO₂R⁶, —NR⁴CONR⁵R⁶ or —SO₂NR⁵R⁶ (wherein R⁴, R⁵ and R⁶ independently represent hydrogen, C₁₋₆ alkyl, —C₃₋₈ cycloalkyl, —C₁₋₆ alkyl-C₃₋₈ cycloalkyl, aryl, heterocyclyl or heteroaryl or —NR⁵R⁶ may represent a nitrogen containing heterocyclyl group, wherein said R⁴, R⁵ and R⁶ groups may be optionally substituted by one or more substituents (e.g. 1, 2 or 3) which may be the same or different, and which are selected from the group consisting of halogen, hydroxy, C₁₋₆ alkyl, C₁₋₆ alkoxy, cyano, amino, ═O or trifluoromethyl); or solvates thereof.

In aspects in which R² is -heteroaryl, -heteroaryl-Y—C₃₋₈ cycloalkyl, -heteroaryl-Y-aryl, -heteroaryl-Y-heteroaryl, -heteroaryl-Y-heterocyclyl, -heterocyclyl-Y—C₃₋₈ cycloalkyl, -heterocyclyl-Y-aryl, -heterocyclyl-Y-heteroaryl or -heterocyclyl-Y-heterocyclyl, R² is linked to A via a carbon atom. In other words, the atom in the heteroaryl or heterocyclyl group that is linked to A is a carbon atom.

The term ‘C_{x-y} alkyl’ as used herein as a group or a part of the group refers to a linear or branched saturated hydrocarbon group containing from x to y carbon atoms. For example, C₁₋₆ alkyl refers to a linear or branched saturated hydrocarbon group containing from 1 to 6 carbon atoms. Examples of C₁₋₆ alkyl groups include methyl, ethyl, n-propyl, isopropyl, n-butyl, isobutyl, sec-butyl, tert butyl, n-pentyl, isopentyl, neopentyl or hexyl and the like.

The term ‘C₂₋₆ alkenyl’ as used herein refers to a linear or branched hydrocarbon group containing one or more carbon-carbon double bonds and having from 2 to 6 carbon atoms. Examples of such groups include ethenyl, propenyl, butenyl, pentenyl or hexenyl and the like.

The term ‘C₁₋₆ alkoxy’ as used herein refers to an —O—C₁₋₆ alkyl group wherein C₁₋₆ alkyl is as defined herein. Examples of such groups include methoxy, ethoxy, propoxy, butoxy, pentoxy or hexoxy and the like.

The term ‘C_{x-y} cycloalkyl’ as used herein refers to a saturated monocyclic hydrocarbon ring of x to y carbon atoms. For example, C₃₋₈ cycloalkyl refers to a saturated monocyclic hydrocarbon ring of 3 to 8 carbon atoms. Examples C₃₋₈ cycloalkyl groups include cyclopropyl, cyclobutyl, cyclopentyl, cyclohexyl, cycloheptyl or cyclooctyl and the like.

The term ‘halogen’ as used herein refers to a fluorine, chlorine, bromine or iodine atom.

The term ‘haloC₁₋₆ alkyl’ as used herein refers to a C₁₋₆ alkyl group as defined herein wherein at least one hydrogen atom is replaced with halogen. Examples of such groups include fluoroethyl, trifluoromethyl or trifluoroethyl and the like.

The term ‘halo C₁₋₆ alkoxy’ as used herein refers to a C₁₋₆ alkoxy group as herein defined wherein at least one hydrogen atom is replaced with halogen. Examples of such groups include difluoromethoxy or trifluoromethoxy and the like.

The term ‘aryl’ as used herein refers to a C₆₋₁₂ monocyclic or bicyclic hydrocarbon ring wherein at least one ring is aromatic. Examples of such groups include phenyl, naphthyl or tetrahydronaphthalenyl and the like.

The term ‘aryloxy’ as used herein refers to an —O-aryl group wherein aryl is as defined herein. Examples of such groups include phenoxy and the like.

The term ‘heteroaryl’ as used herein refers to a 5-6 membered monocyclic aromatic or a fused 8-10 membered bicyclic aromatic ring which monocyclic or bicyclic ring contains 1 to 4 heteroatoms selected from oxygen, nitrogen and sulphur. Examples of such monocyclic aromatic rings include thienyl, furyl, furazanyl, pyrrolyl, triazolyl, tetrazolyl, imidazolyl, oxazolyl, thiazolyl, oxadiazolyl, isothiazolyl, isoxazolyl, thiadiazolyl, pyranyl, pyrazolyl, pyrimidyl, pyridazinyl, pyrazinyl, pyridyl, triazinyl, tetrazinyl and the like. Examples of such fused aromatic rings include quinolinyl, isoquinolinyl, quinazolinyl, quinoxalinyl, pteridinyl, cinnolinyl, phthalazinyl, naphthyridinyl, indolyl, isoindolyl, azaindolyl, indolizinyl, indazolyl, purinyl, pyrrolopyridinyl, furopyridinyl, benzofuranyl, isobenzofuranyl, benzothienyl, benzoimidazolyl, benzoxazolyl, benzoisoxazolyl, benzothiazolyl, benzoisothiazolyl, benzoxadiazolyl, benzothiadiazolyl and the like.

The term ‘heterocyclyl’ refers to a 4-7 membered monocyclic ring or a fused 8-12 membered bicyclic ring which may be saturated or partially unsaturated, which monocyclic or bicyclic ring contains 1 to 4 heteroatoms selected from oxygen, nitrogen or sulphur. Examples of such monocyclic rings include pyrrolidinyl, azetidinyl, pyrazolidinyl, oxazolidinyl, piperidinyl, piperazinyl, morpholinyl, thiomorpholinyl, thiazolidinyl, hydantoinyl, valerolactamyl, oxiranyl, oxetanyl, dioxolanyl, dioxanyl, oxathiolanyl, oxathianyl, dithianyl, dihydrofuranyl, tetrahydrofuranyl, dihydropyranyl, tetrahydropyranyl, tetrahydropyridinyl, tetrahydropyrimidinyl, tetrahydrothiophenyl, tetrahydrothiopyranyl, diazepanyl, azepanyl and the like. Examples of such bicyclic rings include indolinyl, isoindolinyl, benzopyranyl, quinuclidinyl, 2,3,4,5-tetrahydro-1H-3-benzazepine, tetrahydroisoquinolinyl and the like.

The term “nitrogen containing heterocyclyl” is intended to represent any heterocyclyl group as defined above which contains a nitrogen atom.

In one embodiment, R¹ represents:

—C₃₋₇ cycloalkyl (e.g. cyclobutyl, cyclopentyl or cyclohexyl) optionally substituted by a C₁₋₃ alkyl (e.g. methyl) group; or

—C₁₋₆ alkyl (e.g. methyl, ethyl, n-propyl, n-butyl, 2-methyl propyl)

More particularly, R¹ represents unsubstituted —C₃₋₇ cycloalkyl (e.g. cyclobutyl, cyclopentyl or cyclohexyl) or —C₁₋₆ alkyl (e.g. 2-methyl propyl).

In a more particular embodiment, R¹ represents unsubstituted cyclobutyl or cyclopentyl, particularly unsubstituted cyclobutyl.

In another embodiment, A represents a bond or O. In a more particular embodiment A represents a bond.

In a further embodiment, R² represents:

-aryl;

-heteroaryl;

-aryl-Y-heteroaryl;

-heteroaryl-Y-heteroaryl;

-aryl-Y-heterocyclyl; or

-heteroaryl-Y-heterocyclyl.

In one aspect in which the aryl, heteroaryl and heterocyclyl groups of R² are optionally substituted by one or more (e.g. 1, 2 or 3) substituents, the number of cyclic groups comprising the substituted R² group may not exceed three.

In a further aspect, the aryl, heteroaryl and heterocyclyl groups of R² may optionally be substituted by one or more (e.g. 1, 2 or 3) substituents which may be the same or different, and which are selected from the group consisting of halogen, hydroxy, cyano, nitro, ═O, —R⁴ and —CONR⁵R⁶.

In another aspect, Y represents a bond, CO or CONH.

More particularly, R² represents:

-aryl (e.g. -phenyl) optionally substituted by one or more substitutents selected from cyano, —CO₂R⁴ (e.g. —CO₂H) and —CONR⁵R⁶ (e.g. —CONH₂, —CON(H)(Me), —CON(Me)₂, —CONH(Et), —CONH(1-methyl ethyl));

-heteroaryl (e.g. -pyrazinyl, -pyridazinyl, -pyridinyl or imidazolyl) optionally substituted by one or more substitutents selected from halogen (e.g. bromine), cyano, —CO₂R⁴ (CO₂H) and —CONR⁵R⁶ (e.g. —CONH₂, —CON(H)(Me), —CON(Me)₂, —CONH(Et), —CONH(1-methyl ethyl));

-aryl-Y-heteroaryl (e.g. -phenyl-oxadiazolyl, -phenyl-thiadiazolyl, -phenyl-1H-pyrazolyl, -phenyl-1H-triazolyl) optionally substituted by one or more substitutents selected from —R⁴ (e.g. C₁₋₆ alkyl) or ═O groups;

-heteroaryl-Y-heteroaryl (e.g. -pyridinyl-oxadiazolyl, -pyrazinyl-oxadiazolyl) optionally substituted by one or more —R⁴ (e.g. C₁₋₆ alkyl) groups;

-aryl-Y-heterocyclyl (e.g. -phenyl-pyrrolidinyl, -phenyl-CO-pyrrolidinyl, -phenyl-CO-piperidinyl, -phenyl-CO-morpholinyl, -phenyl-CONH-tetrahydro-2H-pyranyl) optionally substituted by one or more ═O groups; or -heteroaryl-Y-heterocyclyl (e.g. pyridinyl-pyrrolidinyl, pyrazinyl-pyrrolidinyl, pyridinyl-imidazolidinyl, pyridinyl-oxazolidinyl, pyridinyl-CO-pyrrolidinyl, pyrazinyl-CO-pyrrolidinyl) optionally substituted by one or more substitutents selected from —R⁴ (e.g. C₁₋₆ alkyl) or =0 groups.

Even more particularly, R² represents:

-aryl (e.g. -phenyl) optionally substituted by one or more substitutents selected from cyano, —CO₂R⁴ (e.g. —CO₂H) and —CONR⁵R⁶ (e.g. —CONH₂, —CON(H)(Me), —CON(Me)₂, —CONH(Et), —CONH(1-methyl ethyl));

-heteroaryl (e.g. -pyrazin-2-yl, -pyridazin-3-yl, -pyridin-2-yl, -pyridin-3-yl or imidazol-1-yl) optionally substituted by one or more substitutents selected from halogen (e.g. bromine), cyano, —CO₂R⁴ (CO₂H) and —CONR⁵R⁶ (e.g. —CONH₂, —CON(H)(Me), —CON(Me)₂, —CONH(Et), —CONH(1-methyl ethyl));

-aryl-Y-heteroaryl (e.g. -phenyl-1,2,4-oxadiazol-5-yl, -phenyl-1,2,3-thiadiazol-4-yl, -phenyl-1H-pyrazol-1-yl, -phenyl-1H-triazol-1-yl) optionally substituted by one or more substitutents selected from —R⁴ (e.g. C₁₋₆ alkyl) or ═O groups (e.g. -phenyl-1,2,4-oxadiazol-5(2H)-one);

-heteroaryl-Y-heteroaryl (e.g. -pyridin-3-yl-1,2,4-oxadiazol-5-yl, -pyridin-2-yl-1,2,4-oxadiazol-5-yl or -pyrazin-2-yl-1,2,4-oxadiazol-5-yl) optionally substituted by one or more C₁₋₆ alkyl (e.g. methyl);

-aryl-Y-heterocyclyl (e.g. -phenyl-pyrrolidin-1-yl, -phenyl-CO-pyrrolidin-1-yl, -phenyl-CO-piperidin-1-yl, -phenyl-CO-morpholin-4-yl, -phenyl-CONH-tetrahydro-2H-pyran-4-yl) optionally substituted by one or more ═O groups (e.g. -phenyl-1-pyrrolidin-2-one); or

-heteroaryl-Y-heterocyclyl (e.g. pyridin-2-yl-pyrrolidinyl, pyridin-3-yl-pyrrolidinyl, pyrazin-2-yl-pyrrolidinyl, pyridin-3-yl-imidazolidinyl, pyridin-3-yl-1,3-oxazolidinyl, pyridin-2-yl-CO-pyrrolidinyl, pyrazin-2-yl-CO-pyrrolidinyl) optionally substituted by one or more substitutents selected from C₁₋₆ alkyl (e.g. methyl) or ═O groups (e.g. -pyridin-2-yl-1-pyrrolidin-2-one, -pyrazin-2-yl-1-pyrrolidin-2-one, -pyridin-3-yl-1-(3-methyl-2-imidazolidinone), -pyridin-3-yl-1,3-oxazolidin-2-one).

More particularly, R² represents -heteroaryl (e.g. -pyrazin-2-yl) optionally substituted by one or more —CONR⁵R⁶ (e.g. —CON(H)(Me), —CONH(Et)) groups.

Most particularly, R² represents N-methyl-2-pyrazinecarboxamide or N-ethyl-2-pyrazinecarboxamide.

In embodiments in which R² represents a monosubstituted 6 membered monocyclic aryl or a 6 membered monocyclic heteroaryl, the substitutent may be attached in the position para to the attachment of the aryl or heteroaryl to A.

In embodiments in which R² represents a 6 membered monocyclic aryl or a 6 membered monocyclic heteroaryl linked to a heteroaryl or heterocyclyl group through Y, the —Y-heteroaryl or —Y-heterocyclyl group may be attached in the para position.

In embodiments in which R² represents an -aryl-Y-heterocyclyl group or a -heteroaryl-Y-heterocyclyl group, and in which the heterocyclyl is a nitrogen containing heterocyclyl, the nitrogen containing heterocyclyl is linked to Y through a nitrogen atom.

In one embodiment, R⁴ represents H or C₁₋₆alkyl (e.g. methyl).

In another embodiment R⁶ represents H or C₁₋₆alkyl (e.g. methyl, ethyl, 1-methyl ethyl).

In a further embodiment R⁶ represents H or C₁₋₆alkyl (e.g. methyl, ethyl, 1-methyl ethyl).

In another embodiment, n represents 0 or 1, more particularly 0.

When n represents 1, R³ may represent a halogen (e.g. iodine) atom or a cyano group.

In one aspect, the invention provides a compound of formula (I) or a pharmaceutically acceptable salt or solvate thereof, wherein:

R¹ represents unsubstituted —C₃₋₇ cycloalkyl;

A represents a bond or 0;

R² represents -aryl, -heteroaryl, -aryl-Y-heteroaryl, -aryl-Y-heterocyclyl, -heteroaryl-Y-aryl, -heteroaryl-Y-heteroaryl, -heteroaryl-Y-heterocyclyl, such that R² is linked to A via a carbon atom;

Y represents a bond, CO or CONH;

n is 0;

wherein said aryl, heteroaryl and heterocyclyl groups of R² may be optionally substituted by one or more substituents (e.g. 1, 2 or 3) which may be the same or different, and which are selected from the group consisting of halogen, hydroxy, cyano, nitro, ═O, —R⁴ and —CONR⁵R⁶; and

wherein R⁴, R⁵ and R⁶ independently represent H or C₁₋₆alkyl.

Compounds according to the invention include examples E1-E60 as shown below, or a pharmaceutically acceptable salt thereof.

More particularly, compounds according to the invention include: 5-[(3-Cyclobutyl-2,3,4,5-tetrahydro-1H-3-benzazepin-7-yl)methyl]-N-methyl-2-pyrazinecarboxamide; or

5-[(3-Cyclobutyl-2,3,4,5-tetrahydro-1H-3-benzazepin-7-yl)methyl]-N-ethyl-2-pyrazinecarboxamide.

Compounds of formula (I) may form acid addition salts with acids, such as conventional pharmaceutically acceptable acids, for example maleic, hydrochloric, hydrobromic, phosphoric, acetic, fumaric, salicylic, sulphate, citric, lactic, mandelic, tartaric and methanesulphonic. Salts, solvates and hydrates of compounds of formula (I) therefore form an aspect of the invention.

A pharmaceutically acceptable acid addition salt can be formed by reaction of the free base with a suitable inorganic or organic acid (such as hydrobromic, hydrochloric, sulfuric, nitric, phosphoric, succinic, maleic, formic, acetic, propionic, fumaric, citric, tartaric, lactic, benzoic, salicylic, glutamaic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated for example by crystallisation and filtration.

The invention includes within its scope all possible stoichiometric and non-stoichiometric forms of the salts of the compounds of the invention including hydrates and solvates.

Certain compounds of formula (I) are capable of existing in stereoisomeric forms. It will be understood that the invention encompasses all geometric and optical isomers of these compounds and the mixtures thereof including racemates. Tautomers also form an aspect of the invention.

The present invention also provides a process for the preparation of a compound of formula (I) or a pharmaceutically acceptable salt thereof, which process comprises:
(a) reacting a compound of formula (II) 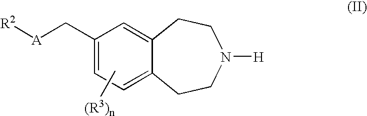
wherein R², R³, A and n are as defined above, with a compound of formula R^{1′}-L¹, wherein R^{1′} is as defined above for R¹ or a group convertible thereto and L¹ represents a suitable leaving group such as a halogen atom (e.g. bromine, iodine or tosylate);
(b) reacting a compound of formula (II) as defined above, with a ketone of formula R^{1″}=O, wherein R^{1″} is =C₁₋₆ alkyl or =C₃₋₇ cycloalkyl, wherein the C₃₋₇ cycloalkyl group may optionally be substituted by C₁₋₃ alkyl;
(c) hydrolysis decarboxylation of a compound of formula (XI) in which A is a bond and R² is -heteroaryl, -heteroaryl-Y-aryl, -heteroaryl-Y-heteroaryl, -heteroaryl-Y-heterocyclyl or heteroaryl-Y—C₃₋₈cycloalkyl, wherein the heteroaryl ring attached to A contains a nitrogen atom ortho to the carbon bonded to A 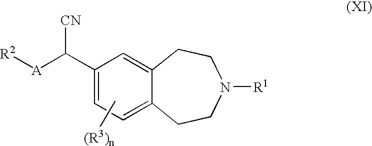
wherein R¹, R³ and n are as defined above;
(d) deprotecting a compound of formula (I) which is protected; or
(e) interconversion from one compound of formula (I) to another.

Process (a) typically comprises the use of a suitable base, such as potassium carbonate in an appropriate solvent such as 2-butanone optionally in the presence of a catalyst such as potassium iodide at an appropriate temperature such as reflux.

Process (b) typically comprises the use of reductive conditions (such as treatment with a borohydride e.g. sodium triacetoxyborohydride), optionally in the presence of an acid, such as acetic acid, in an appropriate solvent such as dichloromethane at a suitable temperature such as room temperature.

Process (c) involves reaction of the compound of formula (XI) in the presence of an acid (e.g. aqueous hydrogen bromide) at a suitable temperature, such as reflux, followed by neutralisation and treatment with a base (e.g. aqueous sodium hydroxide).

In process (d), examples of protecting groups and the means for their removal can be found in T. W. Greene ‘Protective Groups in Organic Synthesis’ (J. Wiley and Sons, 1991). Suitable amine protecting groups include sulphonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid in dioxan or trifluoroacetic acid in dichloromethane) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid.

Process (e) may be performed using conventional interconversion procedures such as epimerisation, oxidation, reduction, alkylation, nucleophilic or electrophilic aromatic substitution, ester or nitrile hydrolysis, amide bond formation or transition metal mediated coupling reactions. Examples of transition metal mediated coupling reactions useful as interconversion procedures include the following: Palladium catalysed coupling reactions between organic electrophiles, such as aryl halides, and organometallic reagents, for example boronic acids (Suzuki cross-coupling reactions); Palladium catalysed amination and amidation reactions between organic electrophiles, such as aryl halides, and nucleophiles, such as amines and amides; Copper catalysed amidation reactions between organic electrophiles (such as aryl halides) and nucleophiles such as amides; and Copper mediated coupling reactions between phenols and boronic acids.

Compounds of formula (II) wherein A represents a bond may be prepared in accordance with the following scheme: 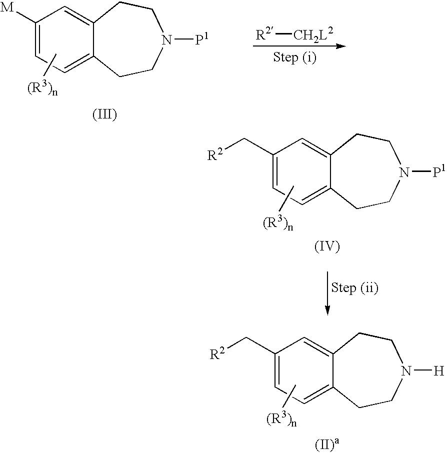
wherein R², R³ and n are as defined above, R^{2′} is as defined above for R² or a group convertible thereto, P¹ represents a suitable protecting group such as Boc, M represents a metal species used in cross-coupling reactions, for example an Sn, B or Zn metal and L² represents a leaving group such as a halide, for example bromine.

Step (i) may be performed under palladium catalysis using standard conditions suitable for the coupling of organic electrophiles, such as a benzyl halide with an organometallic reagent for example a boronic acid. When the organometallic reagent is a boronic acid or boronic ester (Suzuki cross-coupling reaction) the cross-coupling reaction may be performed using a palladium catalyst such as tetrakis(triphenylphosphine)palladium (0), in the presence of a suitable base, for example aqueous sodium carbonate, in a suitable solvent, such as toluene at a suitable temperature, such as reflux. When the organometallic reagent is a organostannane (Stille reaction) or organozinc species the cross-coupling reaction may be performed using a palladium catalyst such as tetrakis(triphenylphosphine)palladium (0), in a suitable solvent, such as toluene or dimethylformamide, at a suitable temperature, such as reflux.

Step (ii) typically comprises a deprotection reaction and may be performed in an analogous manner to that described for process (d), for example, when P¹ represents trifluoroacetyl the deprotection reaction comprises a base catalysed hydrolysis reaction.

Compounds of formula (II) wherein A represents O, S or NR⁷ may be prepared in accordance with the following scheme: 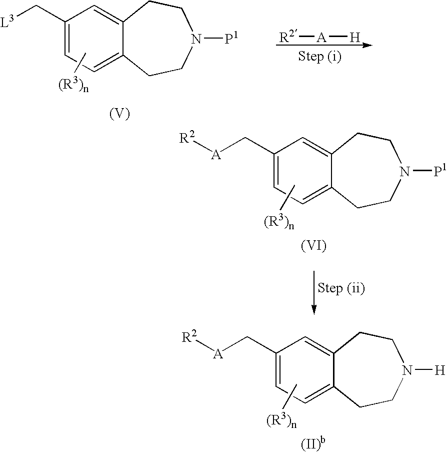
wherein R², R², R³, n and P¹ are as defined above and L³ represents a leaving group such as a halide or triflate group.

Step (i) may be performed in an analogous manner to that described for process (a).

Step (ii) typically comprises a deprotection reaction and may be performed in an analogous manner to that described for process (d).

It would be appreciated by those skilled in the art that when A represents NR⁷ and R⁷ represents hydrogen, a compound of formula (VI) may also be protected with a protecting group which may be selectively removed in preference to P¹ thereby providing a protected compound of formula (II) requiring an additional deprotection reaction.

Compounds of formula (III) wherein M is boron may be prepared in an analogous manner to those described in WO 2004/056369A1.

Compounds of formula (IV) may be prepared in accordance with the following scheme: 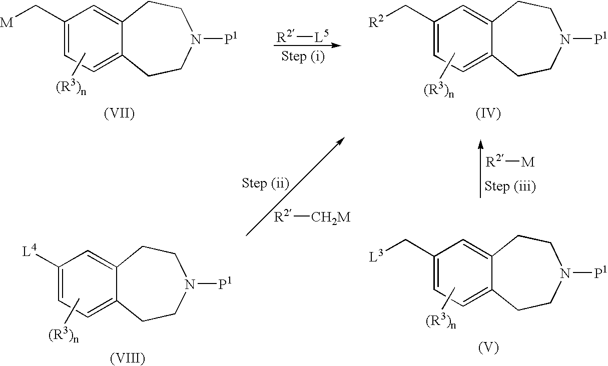
wherein R², R², R³M, n, P¹ and L³ are as defined above and L⁴ and L⁵ independently represent a leaving group such as a halide or triflate group.

Formation of a compound of formula (IV) from a compound of formula (V), (VII) and (VIII) may be performed under palladium catalysis using standard conditions suitable for the coupling of organic electrophiles, such as aryl halides and aryl triflates with an organometallic reagent for example a boronic acid. When the organometallic reagent is a boronic acid or boronic ester (Suzuki cross-coupling reaction) the cross-coupling reaction may be performed using a palladium catalyst such as tetrakis(triphenylphosphine)palladium (0), in the presence of a suitable base, for example aqueous sodium carbonate, in a suitable solvent, such as toluene at a suitable temperature, such as reflux. When the organometallic reagent is an organostannane (Stille reaction) or organozinc species the cross-coupling reaction may be performed using a palladium catalyst such as tetrakis(triphenylphosphine)palladium (0), in a suitable solvent, such as toluene or dimethylformamide, at a suitable temperature, such as reflux.

Compounds of formula (V) and formula (VII) may be prepared according to the following scheme: 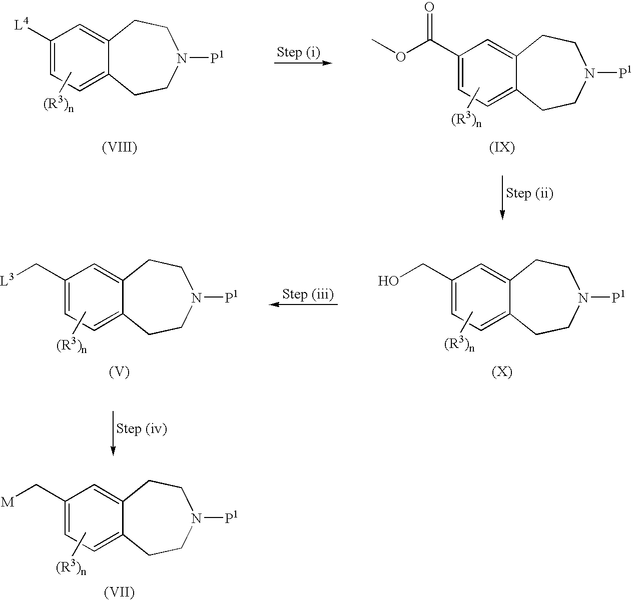
wherein R³, n, P¹, L³ and L⁴ are as defined above.

Step (i) may be performed via a palladium catalysed carbonylation reaction, for example using palladium X, in an atmosphere of carbon monoxide in a suitable solvent, for example dimethylformamide or dimethylsulfoxide, in the presence of methanol, at a suitable temperature, such as with heating.

Step (ii) may be performed under reducing conditions, for example using LiAlH₄ in a suitable solvent, for example ether, at a suitable temperature.

Step (iii), the transformation of an alcohol into a leaving group such as a halogen, for example bromine or a mesylate group according to process (e).

Step (iv) may be performed via reacting a compound of formula (V) wherein L³ is a halogen via a lithium halogen exchange reaction using an organolithium such as butyllithium, followed by quenching of this species with either a boron electrophile, such as trimethylborate, a tin electrophile, such as trimethyltin chloride or a zinc electrophile, for example zinc bromide. Suitable solvents for such reactions include tetrahydrofuran and diethylether.

Compounds of formula (VIII) in which L⁴ is a triflate group may be prepared as outlined in Bioorg. Med. Chem. Lett.; 10; 22; 2000; 2553-2556.

Compounds of formula (XI) may be prepared as outlined in the following scheme. 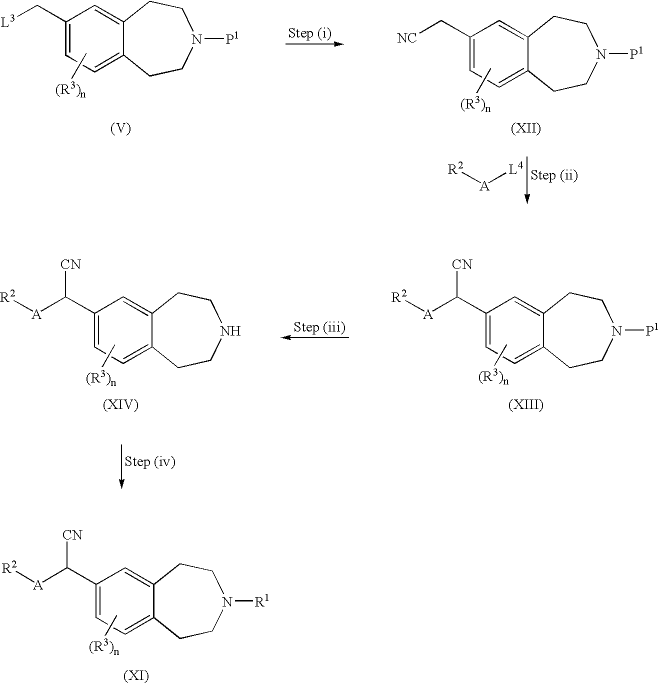
wherein R¹, R³, n, P¹ are as defined above and L³ and L⁴ represent leaving groups, such as bromine and R² is a heteroaryl group and A represents a bond wherein the point of attachment to A is adjacent to a nitrogen atom, for example, R² is a 2-pyridyl group.

Step (i) may be performed using an inorganic cyanide salt, for example, sodium cyanide, in a suitable solvent, for example ethanol, at an appropriate temperature, for example reflux.

Step (ii) may be performed using a base, for example, sodium hydride, in a suitable solvent, for example N,N-dimethylformamide, at an appropriate temperature, for example room temperature.

Step (iii) may be performed according to process (d).

Step (iv) may be performed according to process (a) or (b).

Compounds of formula (I) and their pharmaceutically acceptable salts have affinity for and are antagonists and/or inverse agonists of the histamine H3 receptor and are believed to be of potential use in the treatment of neurological diseases including Alzheimer's disease, dementia, age-related memory dysfunction, mild cognitive impairment, cognitive deficit, epilepsy, pain of neuropathic origin including neuralgias, neuritis and back pain, and inflammatory pain including osteoarthritis, rheumatoid arthritis, acute inflammatory pain and back pain, migraine, Parkinson's disease, multiple sclerosis, stroke and sleep disorders including narcolepsy; psychiatric disorders including schizophrenia (particularly cognitive deficit of schizophrenia), attention deficit hypereactivity disorder, depression and addiction; and other diseases including obesity and gastro-intestinal disorders.

It will also be appreciated that compounds of formula (I) are expected to be selective for the histamine H3 receptor over other histamine receptor subtypes, such as the histamine H1 receptor. Generally, compounds of the invention may be at least 10 fold selective for H3 over H1, such as at least 100 fold selective.

Thus the invention also provides a compound of formula (I) or a pharmaceutically acceptable salt thereof, for use as a therapeutic substance in the treatment or prophylaxis of the above disorders, in particular cognitive impairments in diseases such as Alzheimer's disease and related neurodegenerative disorders.

The invention further provides a method of treatment or prophylaxis of the above disorders, in mammals including humans, which comprises administering to the sufferer a therapeutically effective amount of a compound of formula (I) or a pharmaceutically acceptable salt thereof.

In another aspect, the invention provides the use of a compound of formula (I) or a pharmaceutically acceptable salt thereof in the manufacture of a medicament for use in the treatment of the above disorders.

When used in therapy, the compounds of formula (I) are usually formulated in a standard pharmaceutical composition. Such compositions can be prepared using standard procedures.

Thus, the present invention further provides a pharmaceutical composition for use in the treatment of the above disorders which comprises the compound of formula (I) or a pharmaceutically acceptable salt thereof and a pharmaceutically acceptable carrier.

The present invention further provides a pharmaceutical composition which comprises the compound of formula (I) or a pharmaceutically acceptable salt thereof and a pharmaceutically acceptable carrier.

Compounds of formula (I) may be used in combination with other therapeutic agents, for example medicaments claimed to be useful as either disease modifying or symptomatic treatments of Alzheimer's disease. Suitable examples of such other therapeutic agents may be agents known to modify cholinergic transmission such as 5-HT₆ antagonists, M1 muscarinic agonists, M2 muscarinic antagonists or acetylcholinesterase inhibitors. When the compounds are used in combination with other therapeutic agents, the compounds may be administered either sequentially or simultaneously by any convenient route.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colorants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration.

The compound can be sterilised by exposure to ethylene oxide before suspension in a sterile vehicle. Advantageously, a surfactant or wetting agent is included in the composition to facilitate uniform distribution of the compound. The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.1 to 200 mg and even more suitably 1.0 to 200 mg. In one aspect, a suitable unit dose would be 0.1-50 mg. Such unit doses may be administered more than once a day, for example two or three a day. Such therapy may extend for a number of weeks or months.

The following Descriptions and Examples illustrate the preparation of compounds of the invention.

Description 1

### 1,1-Dimethylethyl 7-[(4-cyanophenyl)methyl]-1,2,4,5-tetrahydro-3H-3-benzazepine-3-carboxylate (D1)

To a solution of 4-bromomethyl benzonitrile (100 mg, 0.51 mmol) in ethylene glycol dimethyl ether (1 ml) was added tetrakis(triphenylphosphine)palladium(0) (30 mg, 0.025 mmol), and a solution of (3-{[(1,1-dimethylethyl)oxy]carbonyl}-2,3,4,5-tetrahydro-1H-3-benzazepin-7-yl)boronic acid (WO 2004056369) (222 mg, 0.77 mmol) in a 2:1 mixture of ethylene glycol dimethyl ether and ethanol (1 ml). The reaction was refluxed for 16 hours under argon. The reaction was cooled, diluted with ethyl acetate and water, and filtered through celite. The organic phase washed with water, brine, dried over anhydrous sodium sulfate and concentrated in vacuo. The resulting residue was purified by column chromatography eluting with a mixture of ethyl acetate:pentane: (10:90 to 40:60) to afford the title product. MS (AP+) m/e 263 [M-COOBu^{t}]⁺.

Description 2

### 4-(2,3,4,5-Tetrahydro-1H-3-benzazepin-7-ylmethyl)benzonitrile (D2)

To a solution of 1,1-dimethylethyl 7-[(4-cyanophenyl)methyl]-1,2,4,5-tetrahydro-3H-3-benzazepine-3-carboxylate (may be prepared as described in Description 1) (162 mg, 0.45 mmol) in dichloromethane (2 ml) was added trifluoroacetic acid (2 ml) and the reaction stirred for 2 hours at room temperature. The reaction mixture was concentrated in vacuo and re-dissolved in dichloromethane (5 ml), and this solution added to 0.880 ammonia (5 ml). The organic phase was separated and the aqueous phase was extracted with dichloromethane (×2). The combined organic phase washed with water, dried over anhydrous sodium sulfate and concentrated in vacuo. The residue was purified by column chromatography eluting with a mixture of 0.880 ammonia:methanol:dichloromethane (1:9:90) to afford the title product. MS (AP+) m/e 263 [M+H]⁺.

Description 3

### 3-(1,1-Dimethylethyl) 7-methyl 1,2,4,5-tetrahydro-3H-3-benzazepine-3,7-dicarboxylate (D3)

Method A

Argon was bubbled through a mixture of 1,1-dimethylethyl 7-{[(trifluoromethyl)sulfonyl]oxy}-1,2,4,5-tetrahydro-3H-3-benzazepine-3-carboxylate (10 g; 25.3 mmol) (may be prepared by the method described in Bioorganic and Medicinal Chemistry Letters 10 (22), 2553 (2000)), palladium acetate (0.29 g; 1.3 mmol), 1,3-bis(diphenylphosphino)propane (0.58 g; 1.4 mmol) and triethylamine (2.0 ml; 27.8 mmol) in dimethylsulfoxide (45 ml) and methanol (30 ml) for 30 minutes. Carbon monoxide was bubbled through the mixture for 25 minutes and the mixture heated at 70° C. under an atmosphere of carbon monoxide for 5 hours. The mixture was filtered through celite and the methanol removed by evaporation under reduced pressure. The residue was poured into water and extracted with ethyl acetate. The extracts were combined, dried (magnesium sulfate) and evaporated. This residue was purified by silica gel chromatography eluting with a mixture of pentane:ethyl acetate (20:1) to afford the title compound (D3) (3.0 g; 38%); MS (AP+) m/e 309 [M+H]⁺.

Method B

Argon was bubbled through a mixture of 1,1-dimethylethyl 7-{[(trifluoromethyl)sulfonyl]oxy}-1,2,4,5-tetrahydro-3H-3-benzazepine-3-carboxylate (30 g; 75.9 mmol) (may be prepared by the method described in Bioorganic and Medicinal Chemistry Letters 10 (22), 2553 (2000)), palladium acetate (851 mg; 3.8 mmol), 1,3-bis(diphenylphosphino)propane (4.2 g; 7.6 mmol) and triethylamine (21 ml; 152 mmol) in dimethylformamide (150 ml) and methanol (60 ml) for 90 minutes. Carbon monoxide was bubbled through the mixture for 30 minutes and the mixture heated at 65° C. under a carbon monoxide filled balloon for 4 hours. The solvent removed by evaporation to give a black gum which was purified on a 75+M biotage column eluting with a mixture of pentane:ethyl acetate (9:1) to afford the title compound as a pale yellow viscous oil. ¹H NMR (CDCl₃) δ 7.80 (2H, m), 7.18 (H, m), 3.90 (3H, s), 3.56 (4H, m), 2.95 (4H, m), 1.48 (9H, s).

Description 4

### 1,1-Dimethylethyl 7-(hydroxymethyl)-1,2,4,5-tetrahydro-3H-3-benzazepine-3-carboxylate (D4)

Method A

A solution of 3-(1,1-dimethylethyl) 7-methyl 1,2,4,5-tetrahydro-3H-3-benzazepine-3,7-dicarboxylate (may be prepared by the method described in Description 3) (0.8 g; 2.6 mmol) in diethyl ether (5 ml) was added to a 1.0M solution of lithium aluminium hydride in diethyl ether (2.6 ml: 2.6 mmol). The resulting mixture was stirred at reflux for 6 hours. The mixture was allowed to cool and was quenched with water. The layers were separated and the aqueous portion extracted with diethyl ether. The extracts were combined, dried (magnesium sulfate) and evaporated. This residue was purified by silica gel chromatography eluting with a mixture of pentane:ethyl acetate (20:1) to afford the title compound (D4) as a colourless oil (0.49 g; 38%); MS (AP+) m/e 204 [M-OtBu]⁺.

Method B

A 1M solution of lithium aluminium hydride in tetrahydrofuran (70 ml, 70 mmol) was cooled in ice/water and a solution of 3-(1,1-dimethylethyl) 7-methyl 1,2,4,5-tetrahydro-3H-3-benzazepine-3,7-dicarboxylate (may be prepared by the method described in Description 3, method B) (10.29 g, 33.7 mmol) in dry diethyl ether (70 ml) was added drop-wise under argon. After addition was complete, the mixture was allowed to warm to room temperature and stirred at room temperature for the weekend. Water (˜10 ml) was added dropwise (caution, vigorous reaction) followed by 1M sodium hydroxide solution. The mixture was diluted with water (˜500 ml) and extracted with diethyl ether (×5). The extracts were combined, washed (2×100 ml water, 100 ml brine), dried (sodium sulphate) and evaporated to afford the title compound as a pale yellow viscous gum. MS (AP+) m/e 204 [M-OtBu]⁺. H¹ NMR (CDCl₃) δ 7.13-7.07 (3H, m), 4.64 (2H, s), 3.51 (4H, m), 2.92 (4H, m), 2.56 (H, br s), 1.49 (9H, s).

Description 5

### 1,1-Dimethylethyl 7-{[(methylsulfonyl)oxy]methyl}-1,2,4,5-tetrahydro-3H-3-benzazepine-3-carboxylate (D5)

Methanesulfonyl chloride (0.11 g; 0.95 mmol) was added to a mixture of 1,1-dimethylethyl 7-(hydroxymethyl)-1,2,4,5-tetrahydro-3H-3-benzazepine-3-carboxylate (may be prepared by the method described in Description 4) (0.24 g; 0.9 mmol) and triethylamine (0.12 ml; 0.9 mmol) in dichloromethane (5 ml) and the mixture stirred at room temperature for 18 hours. The mixture was evaporated to furnish the crude title compound (D5) which may be used directly without purification.

Description 6

### 1,1-Dimethylethyl 7-[({5-[(methyloxy)carbonyl]-2-pyrazinyl}oxy)methyl]-1,2,4,5-tetrahydro-3H-3-benzazepine-3-carboxylate (D6)

A mixture of 1,1-dimethylethyl 7-{[(methylsulfonyl)oxy]methyl}-1,2,4,5-tetrahydro-3H-3-benzazepine-3-carboxylate (may be prepared by the method described in Description 5) (0.9 mmol), methyl 5-oxo-4,5-dihydro-2-pyrazinecarboxylate (0.22 g; 1.4 mmol) (may be prepared by the method described in Synlett (1994), (10), 814-16) and caesium carbonate (0.92 g; 2.8 mmol) in dimethylformamide (3 ml) was heated at 80° C. for 2 hours. The mixture was poured into water and extracted with ethyl acetate. The extracts were combined, dried (magnesium sulfate) and evaporated. This residue was purified by silica gel chromatography eluting with a mixture of pentane:ethyl acetate (1:1) to afford the title compound (D6) as a yellow solid (0.23 g; 39%); MS (AP+) m/e 314 [M-BOC]⁺.

Description 7

### 5-{[(3-{[(1,1-Dimethylethyl)oxy]carbonyl}-2,3,4,5-tetrahydro-1H-3-benzazepin-7-yl)methyl]oxy}-2-pyrazinecarboxylic acid (D7)

Sodium hydroxide (64 mg; 1.6 mmol) was added to a solution of 1,1-dimethylethyl 7-[({5-[(methyloxy)carbonyl]-2-pyrazinyl}oxy)methyl]-1,2,4,5-tetrahydro-3H-3-benzazepine-3-carboxylate (may be prepared by the method described in Description 6) (0.22 g; 0.53 mmol) in methanol (5 ml) and water (2 ml). This mixture was heated at reflux for 30 minutes and the solvent removed by evaporation under reduced pressure. The residue was acidified using 2M hydrochloric acid and extracted with ethyl acetate. The extracts were combined, dried (magnesium sulfate) and evaporated to give the title compound (D7) (0.21 g; 100%); MS (AP+) m/e 344 [M-OtBu]⁺.

Description 8

### 1,1-Dimethylethyl 7-[({5-[(methylamino)carbonyl]-2-pyrazinyl}oxy)methyl]-1,2,4,5-tetrahydro-3H-3-benzazepine-3-carboxylate (D8)

1,1′-(Oxomethanediyl)bis-1H-imidazole (0.17 g; 1.06 mmol) was added to a solution of 5-{[(3-{[(1,1-dimethylethyl)oxy]carbonyl}-2,3,4,5-tetrahydro-1H-3-benzazepin-7-yl)methyl]oxy}-2-pyrazinecarboxylic acid (may be prepared by the method described in Description 7) (0.21 g; 0.53 mmol) in tetrahydrofuran (5 ml). After stirring at room temperature for 18 hours a 2M solution of methylamine (0.53 ml; 1.06 mmol) was added and the mixture stirred at room temperature for 2 hours. The solvent was removed by evaporation and the residue purified by silica gel chromatography eluting with a mixture of dichloromethane:methanol (97:3) to afford the title compound (D8) (0.22 g; 100%) MS (AP+) m/e 313 [M-BOC]⁺.

Description 9

### N-Methyl-5-[(2,3,4,5-tetrahydro-1H-3-benzazepin-7-ylmethyl)oxy]-2-pyrazinecarboxamide (D9)

Trifluoroacetic acid (2 ml) was added to a solution of 1,1-dimethylethyl 7-[({5-[(methylamino)carbonyl]-2-pyrazinyl}oxy)methyl]-1,2,4,5-tetrahydro-3H-3-benzazepine-3-carboxylate (may be prepared by the method described in Description 8) (0.21 mmol; 0.5 mmol) in dichloromethane (5 ml) and the mixture stirred at room temperature for 1 hour. The mixture was evaporated and purified on an SCX ion exchange cartridge eluting with methanol and then 2M ammonia in methanol. Fractions containing the product were combined and evaporated to give the title compound (D9) (0.1 g; 64%) MS (AP+) m/e 313 [M+H]⁺.

Description 10

### 1,1-Dimethylethyl 7-[({5-[(methyloxy)carbonyl]-2-pyridinyl}oxy)methyl]-1,2,4,5-tetrahydro-3H-3-benzazepine-3-carboxylate (D10)

A mixture of 1,1-dimethylethyl 7-{[(methylsulfonyl)oxy]methyl}-1,2,4,5-tetrahydro-3H-3-benzazepine-3-carboxylate (may be prepared as described in Description 5) (450 mg; 1.27 mmol), methyl 6-hydroxy-3-pyridinecarboxylate (291 mg; 1.91 mmol, may be prepared using the method described in Synthesis (3), 285-293 (1995)) and caesium carbonate (828 mg; 2.54 mmol) in dimethylformamide (5 ml) was heated at 80° C. under argon for 2 hours. The mixture was allowed to cool and was poured into water. This was extracted with ethyl acetate (×3) and the extracts combined. These were dried (magnesium sulphate) and evaporated under reduced pressure. The residue was purified by column chromatography on silica eluting with a 20-50% gradient of ethyl acetate in pentane to afford the title compound. MS (ES+) m/e 357 [M-^{t}Bu]⁺

Description 11

### 6-{[(3-{[(1,1-Di methylethyl)oxy]carbonyl}-2,3,4,5-tetrahydro-1H-3-benzazepin-7-yl)methyl]oxy}-3-pyridinecarboxylic acid (D11)

A solution of sodium hydroxide (119 mg, 2.98 mmol) in water (2 ml) was added to a solution of 1,1-dimethylethyl 7-[({5-[(methyloxy)carbonyl]-2-pyridinyl}oxy)methyl]-1,2,4,5-tetrahydro-3H-3-benzazepine-3-carboxylate (may be prepared as described in Description 11) (410 mg, 0.99 mmol) in methanol (6 ml) and the resulting mixture was heated at 90° C. for 30 minutes. The mixture was allowed to cool and evaporated under reduced pressure. The residue was acidified using 2M hydrochloric acid and extracted with ethyl acetate (×3). The extracts were combined, dried (magnesium sulphate) and evaporated under reduced pressure to afford the title compound. MS (AP−) m/e 397 [M-]

Description 12

### 1,1-Dimethylethyl 7-[({5-[(methylamino)carbonyl]-2-pyridinyl}oxy)methyl]-1,2,4,5-tetrahydro-3H-3-benzazepine-3-carboxylate (D12)

A solution of 6-{[(3-{[(1,1-dimethylethyl)oxy]carbonyl}-2,3,4,5-tetrahydro-1H-3-benzazepin-7-yl)methyl]oxy}-3-pyridinecarboxylic acid (may be prepared as described in Description 12) (340 mg, 0.85 mmol) in dry tetrahydrofuran (6 ml) was treated with 1,1′-(oxomethanediyl)bis-1H-imidazole (276 mg, 1.7 mmol) and the mixture was stirred at room temperature under argon overnight. A 2M solution of methylamine in tetrahydrofuran (0.85 ml, 1.7 mmol) was added and stirring continued at room temperature for 2 hours. The mixture was evaporated under reduced pressure and the residue dissolved in ethyl acetate. This solution washed with 0.5M hydrochloric acid and the organic portion dried (magnesium sulphate) and evaporated under reduced pressure to afford the title compound. MS (AP+) m/e 312 [M-BOC]⁺

Description 13

### N-Methyl-6-[(2,3,4,5-tetrahydro-1H-3-benzazepin-7-ylmethyl)oxy]-3-pyridinecarboxamide (D13)

Trifluoroacetic acid (2.5 ml) was added drop wise to a solution of 1,1-dimethylethyl 7-[({5-[(methylamino)carbonyl]-2-pyridinyl}oxy)methyl]-1,2,4,5-tetrahydro-3H-3-benzazepine-3-carboxylate (may be prepared as described in Description 12) (319 mg, 0.78 mmol) in dichloromethane (5 ml) cooled to 0° C. under argon. The resulting mixture was allowed to warm to room temperature and stirred for 1 hour. The mixture was diluted with methanol and purified on a Bondelut SCX ion exchange cartridge eluting with methanol and then 2M ammonia in methanol. The basic fractions were combined and evaporated to afford the title compound. MS (AP+) m/e 312 [M+H]⁺.

Description 14

### 1,1-Dimethylethyl 7-[({6-[(ethyloxy)carbonyl]-3-pyridazinyl}oxy)methyl]-1,2,4,5-tetrahydro-3H-3-benzazepine-3-carboxylate (D14)

A mixture of 1,1-dimethylethyl 7-{[(methylsulfonyl)oxy]methyl}-1,2,4,5-tetrahydro-3H-3-benzazepine-3-carboxylate (may be prepared as described in Description 5) (450 mg; 1.27 mmol), ethyl 6-hydroxy-3-pyridazinecarboxylate (294 mg; 1.91 mmol, may be prepared using the method described in Chem. Pharm. Bull. 42(2), 371-2, (1994)) and caesium carbonate (828 mg; 2.54 mmol) in dimethylformamide (5 ml) was heated at 80° C. under argon for 2 hours. The mixture was allowed to cool to room temperature and was poured into ethyl acetate and water. The aqueous layer was separated and extracted with ethyl acetate (×3). The combined organic fractions were washed with water (×1), dried over magnesium sulphate and evaporated. The residue was purified on Horizon (silica column chromatography) eluting with a 20-50% gradient of ethyl acetate in pentane to afford the title compound as a white solid. MS (ES+) m/e 204 [M-BOC]⁺

Description 15

### 6-{[(3-{[(1,1-Dimethylethyl)oxy]carbonyl}-2,3,4,5-tetrahydro-1H-3-benzazepin-7-yl)methyl]oxy}-3-pyridazinecarboxylic acid (D15)

A solution of sodium hydroxide (104 mg, 2.60 mmol) in water (2 ml) was added to a solution of 1,1-dimethylethyl 7-[({6-[(ethyloxy)carbonyl]-3-pyridazinyl}oxy)methyl]-1,2,4,5-tetrahydro-3H-3-benzazepine-3-carboxylate (may be prepared as described in Description 14) (370 mg, 0.86 mmol) in methanol (6 ml) and the resulting mixture was stirred at 90° C. for 30 minutes under argon. The mixture was allowed to cool and evaporated to remove the methanol. The residue was acidified to pH 1 using 2M hydrochloric acid and extracted with ethyl acetate (×3). The extracts were dried over magnesium sulphate and evaporated to afford the title compound as a white solid. MS (AP−) m/e 398 [M-]

Description 16

### 1,1-Dimethylethyl 7-[({6-[(methylamino)carbonyl]-3-pyridazinyl}oxy)methyl]-1,2,4,5-tetrahydro-3H-3-benzazepine-3-carboxylate (D16)

A stirring solution of 6-{[(3-{[(1,1-dimethylethyl)oxy]carbonyl}-2,3,4,5-tetrahydro-1H-3-benzazepin-7-yl)methyl]oxy}-3-pyridazinecarboxylic acid (may be prepared as described in Description 15) (330 mg, 0.83 mmol) in dry tetrahydrofuran (6 ml) was treated with 1,1′-(oxomethanediyl)bis-1H-imidazole (269 mg, 1.66 mmol) and the mixture was stirred at room temperature under argon overnight. An excess of a 2M solution of methylamine in tetrahydrofuran was added and stirring was continued at room temperature for 2 hours. The tetrahydrofuran was evaporated to give a yellow oil which was dissolved in ethyl acetate (20 ml). This solution washed with 0.5M hydrochloric acid (10 ml) and the organic layer was separated, dried over magnesium sulphate and the solvent evaporated to afford the title compound as a white solid. MS (AP+) m/e 313 [M-BOC]⁺

Description 17

### N-Methyl-6-[(2,3,4,5-tetrahydro-1H-3-benzazepin-7-ylmethyl)oxy]-3-pyridazinecarboxamide (D17)

1,1-dimethylethyl 7-[({6-[(methylamino)carbonyl]-3-pyridazinyl}oxy)methyl]-1,2,4,5-tetrahydro-3H-3-benzazepine-3-carboxylate (may be prepared as described in Description 16) (327 mg, 0.79 mmol) was dissolved in dry dichloromethane (5 ml) and cooled in an ice bath to 0° C. Trifluoroacetic acid (2.5 ml) was added drop wise whilst stirring under argon and the resulting mixture was allowed to warm to room temperature and stirred for 1 hour. The mixture was diluted with methanol and purified on an SCX ion exchange cartridge eluting with methanol and then 2M ammonia in methanol. The basic fractions were combined and evaporated to afford the title compound as a white solid. MS (AP+) m/e 313 [M+H]⁺.

Description 18

### 4-[(3-{[(1,1-Dimethylethyl)oxy]carbonyl}-2,3,4,5-tetrahydro-1H-3-benzazepin-7-yl)methyl]benzoic acid (D18)

1,1-dimethylethyl 7-[(4-cyanophenyl)methyl]-1,2,4,5-tetrahydro-3H-3-benzazepine-3-carboxylate (may be prepared as described in Description 1) (333 mg, 0.92 mmol) was dissolved in ethanol (3 ml), treated with 10% aqueous sodium hydroxide solution (3 ml) and heated under reflux for 4 hours. The reaction mixture was allowed to cool to room temperature and evaporated in vacuo. The residue was diluted with water, acidified with 5M hydrochloric acid and extracted with ethyl acetate (×3). The ethyl acetate layers were combined, dried under magnesium sulphate and evaporated in vacuo to afford the title product. MS (AP+) m/e 282 [[M-COOBu^{t}]+H]⁺.

Description 19

### 1,1-Dimethylethyl 7-{[4-(3-methyl-1,2,4-oxadiazol-5-yl)phenyl]methyl}-1,2,4,5-tetrahydro-3H-3-benzazepine-3-carboxylate (D19)

4-[(3-{[(1,1-Dimethylethyl)oxy]carbonyl}-2,3,4,5-tetrahydro-1H-3-benzazepin-7-yl)methyl]benzoic acid (may be prepared as described in Description 18) (279 mg, 0.73 mmol) was dissolved in tetrahydrofuran (2.7 ml), treated with N,N′-carbonyldiimidazole (130 mg, 0.81 mmol) and heated under reflux for 2 hours. The reaction mixture was allowed to cool to room temperature and the solvent evaporated in vacuo. The residue was dissolved in toluene (3 ml), treated with acetamide oxime (162 mg) and the resulting mixture heated under reflux for 18 hours. The reaction was allowed to cool to room temperature and the solvent evaporated in vacuo. The resulting residue was purified by column chromatography eluting with ethyl acetate:pentane (1:4) to afford the title product. MS (AP+) m/e 320 [[M-COOBu^{t}]+H]⁺.

Description 20

### 7-{[4-(3-Methyl-1,2,4-oxadiazol-5-yl)phenyl]methyl}-2,3,4,5-tetrahydro-1H-3-benzazepine (D20)

1,1-Dimethylethyl 7-{[4-(3-methyl-1,2,4-oxadiazol-5-yl)phenyl]methyl}-1,2,4,5-tetrahydro-3H-3-benzazepine-3-carboxylate (may be prepared as described in Description 19) (120 mg, 0.29 mmol) in dichloromethane (2 ml) was added trifluoroacetic acid (2 ml) and the resulting mixture was stirred for 1 hour at room temperature. The reaction mixture was concentrated in vacuo and the residue dissolved in methanol and applied to an SCX ion exchange cartridge (Varian bond-elute, 5 g) and washed with methanol and 2M ammonia/methanol. The combined basic fractions were concentrated in vacuo to afford the title product. MS (AP+) m/e 320 [M+H]⁺.

Description 21

### 1,1-Dimethylethyl 7-[(6-cyano-3-pyridinyl)methyl]-1,2,4,5-tetrahydro-3H-3-benzazepine-3-carboxylate (D21)

(3-{[(1,1-Dimethylethyl)oxy]carbonyl}-2,3,4,5-tetrahydro-1H-3-benzazepin-7-yl)boronic acid (may be prepared using the general method described in WO 2004056369) (525 mg, 1.80 mmol) and 5-(bromomethyl)-2-pyridinecarbonitrile (may be prepared using the general method described in J. Med. Chem., 2003, 46, 17, 3612-3622) (477 mg, 1.64 mmol) were dissolved in a 5:8 mixture of ethanol and 1,2-dimethoxyethane (13 ml) and treated with 1M aqueous sodium carbonate solution (4.1 ml, 4.1 mmol) and tetrakis(triphenylphosphine)palladium (0) (95 mg, 0.08 mmol). The resulting mixture was heated under reflux overnight. The reaction was cooled to room temperature, diluted with ethyl acetate and filtered through celite. The filtrate washed with water, separated, dried over magnesium sulphate and evaporated. The resulting residue was purified by column chromatography eluting with ethyl acetate:pentane (1:4) to afford the title product. MS (AP+) m/e 264 [[M-COOBu^{t}]+H]⁺.

Description 22

### 5-[(3-{[(1,1-Dimethylethyl)oxy]carbonyl}-2,3,4,5-tetrahydro-1H-3-benzazepin-7-yl)methyl]-2-pyridinecarboxylic acid (D22)

1,1-Dimethylethyl 7-[(6-cyano-3-pyridinyl)methyl]-1,2,4,5-tetrahydro-3H-3-benzazepine-3-carboxylate (may be prepared as described in Description 21) (235 mg, 0.65 mmol) was dissolved in ethanol (2 ml), treated with 10% aqueous sodium hydroxide solution (2 ml) and heated under reflux for 7 hours. The reaction mixture was allowed to cool to room temperature and evaporated in vacuo. The residue was diluted with water, acidified with 5M hydrochloric acid and extracted with ethyl acetate (×2). The ethyl acetate layers were combined, dried under magnesium sulphate and evaporated in vacuo to afford the title product. MS (AP+) m/e 381 [M−H]⁺.

Description 23

### 1,1-Dimethylethyl 7-{[6-(3-methyl-1,2,4-oxadiazol-5-yl)-3-pyridinyl]methyl}-1,2,4,5-tetrahydro-3H-3-benzazepine-3-carboxylate (D23)

5-[(3-{[(1,1-Dimethylethyl)oxy]carbonyl}-2,3,4,5-tetrahydro-1H-3-benzazepin-7-yl)methyl]-2-pyridinecarboxylic acid (may be prepared as described in Description 22) (176 mg, 0.46 mmol) was dissolved in dichloromethane (9.4 ml), treated with N,N′-carbonyldiimidazole (149 mg, 0.92 mmol) and the resulting mixture stirred at room temperature for 18 hours. The solvent was removed in vacuo, the residue redissolved in toluene (3 ml), treated with acetamide oxime (51 mg, 0.69 mmol) and the resulting mixture heated under reflux for 18 hours. The reaction was allowed to cool to room temperature and the solvent evaporated in vacuo. The resulting residue was purified by column chromatography eluting with ethyl acetate:pentane (1:4 to 1:1) to afford the title product. MS (AP+) m/e 421 [M+H]⁺.

Description 24

### 1,1-Dimethylethyl 7-(bromomethyl)-1,2,4,5-tetrahydro-3H-3-benzazepine-3-carboxylate (D24)

A solution of 1,1-dimethylethyl 7-(hydroxymethyl)-1,2,4,5-tetrahydro-3H-3-benzazepine-3-carboxylate (may be prepared as described in Description 4, method B) (5.38 g, 19.4 mmol) in dry dichloromethane (100 ml) was cooled in ice/water and treated with triphenylphosphine (7.6 g, 29.1 mmol) under argon. N-Bromosuccinamide (5.2 g, 29.1 mmol) was added portion-wise and the mixture allowed to warm to room temperature and stirred for 3 hours. The solvent was removed by evaporation and the residue (black gum) was purified on a 75+M biotage cartridge eluting with 9-1 pentane-ethyl acetate. Fractions containing the product were combined and evaporated to afford the title compound as a colourless oil. MS (AP+) m/e 240 & 242 [M-COOtBu]⁺.

Description 25

### 1,1-dimethylethyl 7-(cyanomethyl)-1,2,4,5-tetrahydro-3H-3-benzazepine-3-carboxylate (D25)

To a solution of 1,1-dimethylethyl 7-(bromomethyl)-1,2,4,5-tetrahydro-3H-3-benzazepine-3-carboxylate (may be prepared as described in Description 24) (770 mg, 2.26 mmol) in a mixture of ethanol (3 ml) and water (0.5 ml) was added sodium cyanide (111 mg, 2.26 mmol) and the reaction refluxed for 1 hour. The reaction mixture was cooled and partitioned between ethyl acetate and water, and the organic phase separated and washed with brine and dried over sodium sulphate and evaporated in vacuo to a crude oil which was purified using silica gel chromatography eluting with a mixture of ethyl acetate/pentane to afford the title product as an oil 623 mg, (96%). (MS (AP+): [M-Bu^{t}]⁺ at m/z 231 (C12H14BrNO2 requires [M-Bu^{t}]⁺ at m/z 231).

Description 26

### 1,1-Dimethylethyl 7-[(5-bromo-2-pyridinyl)(cyano)methyl]-1,2,4,5-tetrahydro-3H-3-benzazepine-3-carboxylate (D26)

To an ice cooled suspension of sodium hydride (60 wt % dispersion on mineral oil, 26 mg, 0.66 mmol) in N,N-dimethylformamide (1 ml) was added drop wise a solution of 1,1-dimethylethyl 7-(cyanomethyl)-1,2,4,5-tetrahydro-3H-3-benzazepine-3-carboxylate (may be prepared as described in Description 25) (180 mg, 0.629 mmol) in N,N-dimethylformamide (1 ml) and stirred for 25 minutes. The reaction was then treated with a solution of 2,5-dibromopyridine (223 mg, 0.943 mmol) in N,N-dimethylformamide (1 ml) drop wise over 20 minutes and allowed to warm to room temperature for 1 hour. The solvent was evaporated in vacuo and the residue partitioned between ethyl acetate and water. The aqueous was separated and extracted twice with ethyl acetate. The combined organic phase was washed with water, brine, dried over sodium sulphate and evaporated in vacuo. The crude residue was purified using silica gel chromatography eluting with a mixture of ethyl acetate/pentane to afford the title product 77 mg, (28%). (MS (AP+): [M-Bu^{t}]⁺ at m/z 342/344 (C22H24BrN3O2 requires [M-Bu^{t}]⁺ at m/z 342/344).

Description 27

### (5-bromo-2-pyridinyl)(2,3,4,5-tetrahydro-1H-3-benzazepin-7-yl)acetonitrile (D27)

Trifluoroacetic acid (2 ml) was added to a solution of the product of 1,1-dimethylethyl 7-[(5-bromo-2-pyridinyl)(cyano)methyl]-1,2,4,5-tetrahydro-3H-3-benzazepine-3-carboxylate (may be prepared as described in Description 26) (235 mg, 0.53 mmol) in dichloromethane (3 ml) and the reaction stirred for 30 minutes at room temperature. The solvent was evaporated in vacuo and the residue purified using a Varian Mega Bondelut SCX cartridge eluting with methanol followed by a 2M solution of ammonia in methanol to afford the title product after evaporation 167 mg (92%). [M+H]⁺ at m/z 342/344 (C₁₇H₁₆BrN₃ requires [M+H]⁺ at m/z 342/344).

Description 28

### (5-bromo-2-pyridinyl)(3-cyclobutyl-2,3,4,5-tetrahydro-1H-3-benzazepin-7-yl)acetonitrile (D28)

Cyclobutanone (51 mg, 0.73 mmol) was added to a solution of (5-bromo-2-pyridinyl)(2,3,4,5-tetrahydro-1H-3-benzazepin-7-yl)acetonitrile (may be prepared as described in Description 27) (167 mg, 0.48 mmol) in a mixture of dichloromethane/acetic acid (99:1, v:v). The reaction was stirred at room temperature for 1 hour then treated with sodium triacetoxyborohydride (155 mg, 0.73 mmol) and stirred at room temperature for a further 2 hours. The reaction was diluted with methanol (2 ml), and purified using a Varian Mega Bond Elut SCX cartridge eluting with methanol followed by a 2M solution of ammonia in methanol to afford the title product after evaporation 182 mg (94%). [M+H]⁺ at m/z 396/398 (C21H22BrN3 requires [M+H]⁺ at m/z 396/398).

Description 29

### Methyl 5-(trimethylstannanyl)-2-pyrazinecarboxylate (D29)

To a solution of methyl 5-chloro-2-pyrazinecarboxylate (2.22 g, 12.90 mmol), tetrakis(triphenylphosphine)palladium (0) (0.75 g, 0.65 mmol) and tetrabutylammonium iodide (5.24 g, 14.19 mmol) in toluene was added hexamethylditin (4.65 g, 14.19 mmol) in toluene, such that the total volume of toluene was 60 ml. The resulting mixture was heated under reflux under argon for 30 minutes, allowed to cool to room temperature and the solvent removed under reduced pressure. The resulting residue was purified of a FLASH 75 column eluting with 1:9 ethyl acetate: pentane followed by 1:4 ethyl acetate: pentane to afford the title product. MS (AP+) m/e 302 [M+2H]⁺.

Description 30

### 1,1-Dimethylethyl 7-({5-[(methyloxy)carbonyl]-2-pyrazinyl}methyl)-1,2,4,5-tetrahydro-3H-3-benzazepine-3-carboxylate (D30)

Method A

To a solution of 1,1-dimethylethyl 7-(bromomethyl)-1,2,4,5-tetrahydro-3H-3-benzazepine-3-carboxylate (0.94 g, 2.78 mmol) (may be prepared as described in Description 24) and bis(triphenylphosphine)palladium (II) chloride (98 mg, 0.14 mmol) in dioxane was added methyl 5-(trimethylstannanyl)-2-pyrazinecarboxylate (may be prepared as described in Description 29, 1.0 g, 3.33 mmol) in dioxane, such that the total volume of dioxane was 15 ml. The resulting mixture was heated under reflux under argon for 1.5 hours, allowed to cool to room temperature and the solvent removed under reduced pressure. The resulting residue was purified by column chromatography eluting with ethyl acetate:pentane (1:1) to afford the title product. MS (AP+) m/e 398 [M+H]⁺.

Method B

### 1,1-Dimethylethyl 7-(bromomethyl)-1,2,4,5-tetrahydro-3H-3-benzazepine-3-carboxylate

(300 mg, 0.88 mmol) (may be prepared as described in Description 24), bis(triphenylphosphine)palladium (II) chloride (31 mg, 0.04 mmol) and methyl 5-(trimethylstannanyl)-2-pyrazinecarboxylate (may be prepared as described in Description 29, 318 mg, 1.06 mmol) were added together in dioxane (5 ml). The resulting mixture was heated under reflux under argon for 2 hours, allowed to cool to room temperature and the solvent removed under reduced pressure. The resulting residue was purified by column chromatography eluting with ethyl acetate:pentane (1:1) to afford the title product. MS (AP+) m/e 398 [M+H]⁺.

Description 31

### 5-[(3-{[(1,1-Dimethylethyl)oxy]carbonyl}-2,3,4,5-tetrahydro-1H-3-benzazepin-7-yl)methyl]-2-pyrazinecarboxylic acid (D31)

Method A

1,1-Dimethylethyl 7-({5-[(methyloxy)carbonyl]-2-pyrazinyl}methyl)-1,2,4,5-tetrahydro-3H-3-benzazepine-3-carboxylate (may be prepared as described in Description 30, method A) (846 mg, 2.13 mmol) was dissolved in ethanol (10 ml), treated with 2M aqueous sodium hydroxide solution (3.2 ml, 6.39 mmol) and the resulting mixture was stirred at room temperature for 30 minutes. The solvent was removed under reduced pressure and the residue diluted with water, acidified with 2M hydrochloric acid solution and extracted with ethyl acetate (×2). The ethyl acetate layers were combined, dried and evaporated to afford the title product. MS (AP+) m/e 284 [[M-COOBu^{t}]+H]⁺.

Method B

1,1-Dimethylethyl 7-({5-[(methyloxy)carbonyl]-2-pyrazinyl}methyl)-1,2,4,5-tetrahydro-3H-3-benzazepine-3-carboxylate (may be prepared as described in Description 30, method B) (231 mg, 0.58 mmol) was dissolved in ethanol (4 ml), treated with 2M aqueous sodium hydroxide solution (0.87 ml, 1.75 mmol) and the resulting mixture was stirred for 1 hour. The solvent was removed under reduced pressure and the residue diluted with water, acidified with 2M hydrochloric acid solution and extracted with ethyl acetate (×2). The ethyl acetate layers were combined, dried and evaporated to afford the title product. MS (ES−) m/e 382 [M−H]⁻.

Description 32

### 1,1-Dimethylethyl 7-{[5-(3-methyl-1,2,4-oxadiazol-5-yl)-2-pyrazinyl]methyl}-1,2,4,5-tetrahydro-3H-3-benzazepine-3-carboxylate (D32)

5-[(3-{[(1,1-Dimethylethyl)oxy]carbonyl}-2,3,4,5-tetrahydro-1H-3-benzazepin-7-yl)methyl]-2-pyrazinecarboxylic acid (may be prepared as described in Description 31) (100 mg, 0.26 mmol) was dissolved in dichloromethane (2.5 ml), treated with N,N′-carbonyldiimidazole (85 mg, 0.52 mmol) and the resulting mixture stirred at room temperature for 18 hours. The solvent was removed in vacuo, the residue redissolved in toluene (3 ml), treated with acetamide oxime (58 mg, 0.78 mmol) and the resulting mixture heated under reflux for 36 hours. The reaction was allowed to cool to room temperature and the solvent evaporated in vacuo. The resulting residue was purified by column chromatography eluting with ethyl acetate:pentane (1:1) to afford the title product. MS (AP+) m/e 322 [[M-COOBu^{t}]+H]⁺.

Description 33

### 6-(tributylstannanyl)-3-pyridinecarbonitrile (D33)

To a solution of hexabutyldistannane (3.98 ml, 7.94 mmol) in tetrahydrofuran (40 ml) at 0° C., under argon, was added a 2.5M solution of n-butyllithium in hexanes (3.18 ml, 7.94 mmol). The resulting mixture was allowed to stir at 0° C. for 15 minutes before the addition of a solution of 6-chloro-3-pyridinecarbonitrile (1.00 g, 7.22 mmol) in tetrahydrofuran (5 ml). The reaction mixture was allowed to warm slowly to room temperature overnight. The reaction mixture was evaporated and the residue was purified by chromatography using a 40+M biotage cartridge, eluting with a gradient of ethyl acetate and hexane (0-10%) to afford the title product; MS (ES+) m/e 393, 394, 395 [M+H]⁺.

Description 34

### 1,1-Dimethylethyl 7-[(5-cyano-2-pyridinyl)methyl]-1,2,4,5-tetrahydro-3H-3-benzazepine-3-carboxylate (D34)

A mixture of 6-(tributylstannanyl)-3-pyridinecarbonitrile (may be prepared as described in Description 33) (173 mg, 0.44 mmol), 1,1-dimethylethyl 7-(bromomethyl)-1,2,4,5-tetrahydro-3H-3-benzazepine-3-carboxylate (may be prepared as described in Description 24) (100 mg, 0.29 mmol) and bis(triphenylphosphine)palladium(II) chloride (11.0 mg, 0.015 mmol) in dioxan (4 ml) was heated at reflux overnight. The reaction mixture was evaporated and purified on a 25+M biotage cartridge, eluting with a gradient of ethyl acetate and pentane (10-30%) to afford the title product; MS (ES+) m/e 308 [M-tBu]⁺.

Description 35

### 6-(2,3,4,5-tetrahydro-1H-3-benzazepin-7-ylmethyl)-3-pyridinecarbonitrile (D35)

1,1-dimethylethyl 7-[(5-cyano-2-pyridinyl)methyl]-1,2,4,5-tetrahydro-3H-3-benzazepine-3-carboxylate (may be prepared as described in Description 34) (92.0 mg, 0.25 mmol) was dissolved in dichloromethane (2 ml) and cooled to 0° C. 2 ml trifluoroacetic acid was added and the mixture was stirred for 30 minutes at room temperature. The reaction mixture was then applied to a SCX cartridge (Varian bond-elute, 2 g), eluting with methanol followed by 2M ammonia/methanol. The basic fractions were combined, evaporated and purified further on a 25+5 Biotage cartridge, eluting with gradient of ammonia in methanol/dichloromethane (2-6%) to afford the title compound. MS (ES+) m/e 264 [M+H]⁺.

Description 36

### 6-[(3-{[(1,1-dimethylethyl)oxy]carbonyl}-2,3,4,5-tetrahydro-1H-3-benzazepin-7-yl)methyl]-3-pyridinecarboxylic acid (D36)

A solution of 1,1-dimethylethyl 7-[(5-cyano-2-pyridinyl)methyl]-1,2,4,5-tetrahydro-3H-3-benzazepine-3-carboxylate (may be prepared as described in Description 34) (840 mg, 2.31 mmol) in ethanol (15 ml) was treated with a 10% aqueous sodium hydroxide solution (15 ml). The resulting mixture was heated at reflux for 3 hours and then evaporated to dryness. The residue was redissolved in water (20 ml), acidified with hydrochloric acid and extracted into ethyl acetate (2×40 ml). The combined extracted were dried over magnesium sulphate and evaporated to yield the title compound. MS (ES+) m/e 327 [M-tBu]⁺.

Description 37

### 1,1-Dimethylethyl 7-({5-[(methylamino)carbonyl]-2-pyridinyl}methyl)-1,2,4,5-tetrahydro-3H-3-benzazepine-3-carboxylate (D37)

A mixture of 6-[(3-{[(1,1-dimethylethyl)oxy]carbonyl}-2,3,4,5-tetrahydro-1H-3-benzazepin-7-yl)methyl]-3-pyridinecarboxylic acid (may be prepared as described in Description 36) (100 mg, 0.26 mmol), polymer bound dicyclohexylcarbodiimide resin (248 mg, 0.52 mmol, 2.1 mmol/g) and 1-hydroxybenzotriazole (70.0 mg, 0.52 mmol) and dimethylformamide (2 ml) were stirred at room temperature for 1 hour. Methylamine (40% solution in water, 45.0 μl, 0.52 mmol) was then added and the resulting mixture stirred at room temperature for 2 hours. The reaction mixture was filtered and solvent was removed in vacuo. The product was purified by chromatography on silica, eluting with a gradient of 2M ammonia in methanol/dichloromethane (2-6%) to afford the title compound. MS (ES+) m/e 340 [M-tBu]⁺.

Descriptions 38-39 (D38-39)

Intermediates D38-39 may be prepared from 6-[(3-{[(1,1-dimethylethyl)oxy]carbonyl}-2,3,4,5-tetrahydro-1H-3-benzazepin-7-yl)methyl]-3-pyridinecarboxylic acid (may be prepared as described in Description 36) and the appropriate amine using an analogous method to that described for Description D37 (see table)

| | | LC/MS |
|---|---|---|
| Description | Amine | (M-tBu⁺) |
| 1,1-dimethylethyl 7-{[5- | Pyrrolidine | 380 |
| (1-pyrrolidinylcarbonyl)-2- | | |
| pyridinyl]methyl}-1,2,4,5-tetrahydro-3H-3- | | |
| benzazepine-3-carboxylate (D38) | | |
| 1,1-dimethylethyl 7-{[5-(aminocarbonyl)-2- | ammonia (35% | 326 |
| pyridinyl]methyl}-1,2,4,5-tetrahydro-3H-3- | solution in | |
| benzazepine-3-carboxylate (D39) | water) | |

Description 40

### 1,1-Dimethylethyl 7-{[5-(3-methyl-1,2,4-oxadiazol-5-yl)-2-pyridinyl]methyl}-1,2,4,5-tetrahydro-3H-3-benzazepine-3-carboxylate (D40)

6-[(3-{[(1,1-Dimethylethyl)oxy]carbonyl}-2,3,4,5-tetrahydro-1H-3-benzazepin-7-yl)methyl]-3-pyridinecarboxylic acid (150 mg, 0.39 mmol) (may be prepared as described in Description 36) was dissolved in tetrahydrofuran (1.5 ml), treated with N,N′-carbonyldiimidazole (70 mg, 0.43 mmol) and heated under reflux for 4 hours. The reaction mixture was allowed to cool to room temperature and the solvent evaporated in vacuo. The residue was dissolved in toluene (3 ml), treated with acetamide oxime (87 mg) and the resulting mixture heated under reflux for 54 hours. The reaction was allowed to cool to room temperature and the solvent evaporated in vacuo. The resulting residue was purified by column chromatography eluting with ethyl acetate:pentane (1:4) to afford the title product. MS (AP+) m/e 365 [[M-Bu^{t}]+H]⁺.

Description 41

### 1,1-Dimethylethyl 7-({4-[(dimethylamino)carbonyl]phenyl}methyl)-1,2,4,5-tetrahydro-3H-3-benzazepine-3-carboxylate (D41)

4-[(3-{[(1,1-Dimethylethyl)oxy]carbonyl}-2,3,4,5-tetrahydro-1H-3-benzazepin-7-yl)methyl]benzoic acid (may be prepared as described in Description 18) (216 mg, 0.57 mmol) was dissolved in tetrahydrofuran (3 ml), treated with N,N′-carbonyldiimidazole (101 mg, 0.62 mmol) and heated under reflux for 2 hours. The reaction mixture was allowed to cool to room temperature, stirred for 18 hours and the solvent evaporated in vacuo. The residue was dissolved in dichloromethane (3 ml), treated with 2M dimethylamine in THF (0.29 ml, 0.58 mmol) and the resulting mixture stirred for 4 hours. The reaction mixture was applied directly to a silica column and the product purified by column chromatography eluting with ethyl acetate:pentane (1:1) to afford the title product. MS (AP+) m/e 309 [M-COOBu^{t}]⁺.

Descriptions 42-43 (D42-43)

The following intermediates may be prepared from 4-[(3-{[(1,1-dimethylethyl)oxy]carbonyl}-2,3,4,5-tetrahydro-1H-3-benzazepin-7-yl)methyl]benzoic acid (be prepared as described in Description 18) and the corresponding amine using an analogous method to that described for Description 41.

| Description | Amine | Mass Spectrum |
|---|---|---|
| 1,1-Dimethylethyl 7-{[4-(1- | Piperidine | MS(AP+) m/e |
| piperidinylcarbonyl)phenyl]- | | 349[[M-COOBu^{t}] + H]⁺ |
| methyl}- | | |
| 1,2,4,5-tetrahydro-3H-3- | | |
| benzazepine-3- | | |
| carboxylate (D42) | | |
| 1,1-Dimethylethyl 7-{[4-(4- | Morpholine | MS(AP+) m/e |
| morpholinylcarbonyl)phenyl]- | | 351[[M-COOBu^{t}] + H]⁺ |
| methyl}- | | |
| 1,2,4,5-tetrahydro-3H-3- | | |
| benzazepine-3- | | |
| carboxylate (D43) | | |

Description 44

### 1,1-Dimethylethyl 7-({5-[(methylamino)carbonyl]-2-pyrazinyl}methyl)-1,2,4,5-tetrahydro-3H-3-benzazepine-3-carboxylate (D44)

5-[(3-{[(1,1-Dimethylethyl)oxy]carbonyl}-2,3,4,5-tetrahydro-1H-3-benzazepin-7-yl)methyl]-2-pyrazinecarboxylic acid (may be prepared as described in Description 31, method B) (119 mg, 0.31 mmol) was dissolved in dichloromethane (5 ml), treated with N,N′-carbonyldiimidazole (100 mg, 0.62 mmol) and stirred at room temperature under argon for 18 hours. The reaction mixture was treated with 2M methylamine in THF (0.62 ml, 1.24 mmol) and the resulting mixture stirred at room temperature under argon for 2 hours. The reaction mixture was applied directly to a column and the product purified by column chromatography eluting with ethyl acetate:pentane (1:1) to afford the title product. MS (AP+) m/e 397 [M+H]⁺.

Descriptions 45-46 (D45-46)

The following intermediates may be prepared from 5-[(3-{[(1,1-dimethylethyl)oxy]carbonyl}-2,3,4,5-tetrahydro-1H-3-benzazepin-7-yl)methyl]-2-pyrazinecarboxylic acid (may be prepared as described in Description 31) and the corresponding amine using an analogous method to that described for Description 44.

| Description | Amine | Mass Spectrum |
|---|---|---|
| 1,1-Dimethylethyl 7-{[5-(1- | Pyrrolidine | MS(AP+) m/e |
| pyrrolidinylcarbonyl)-2- | | 437[M + H]⁺ |
| pyrazinyl]methyl}-1,2,4,5- | | |
| tetrahydro-3H-3-benzazepine- | | |
| 3-carboxylate (D45) | | |
| 1,1-Dimethylethyl 7-[(5-{[(1- | Isopropylamine | MS(AP+) m/e |
| methylethyl)amino]carbonyl}-2- | | 425[M + H]⁺ |
| pyrazinyl)methyl]-1,2,4,5- | | |
| tetrahydro-3H-3-benzazepine- | | |
| 3-carboxylate (D46) | | |

Description 47

### 1,1-Dimethylethyl 7-({5-[(ethylamino)carbonyl]-2-pyrazinyl}methyl)-1,2,4,5-tetrahydro-3H-3-benzazepine-3-carboxylate (D47)

Step 1

5-[(3-{[(1,1-Dimethylethyl)oxy]carbonyl}-2,3,4,5-tetrahydro-1H-3-benzazepin-7-yl)methyl]-2-pyrazinecarboxylic acid (may be prepared as described in Description 31, method A) (400 mg, 1.04 mmol) was dissolved in dichloromethane (10 ml), treated with N,N′-carbonyldiimidazole (338 mg, 2.08 mmol) and stirred at room temperature for 18 hours.

Step 2

One quarter of the product of Description 47, step 1 (0.2 mmol) dissolved in 3 ml dichloromethane was treated with ethylamine hydrochloride (65 mg, 0.80 mmol) and triethylamine (0.11 ml, 0.80 mmol) and the resulting mixture stirred for 2 hours at room temperature. The reaction mixture was applied directly to a column and the product purified by eluting with ethyl acetate:pentane (1:1) to afford the title product. MS (AP+) m/e 411 [M+H]⁺.

Descriptions 48-50 (D48-50)

The following intermediates may be prepared from 5-[(3-{[(1,1-dimethylethyl)oxy]carbonyl}-2,3,4,5-tetrahydro-1H-3-benzazepin-7-yl)methyl]-2-pyrazinecarboxylic acid (may be prepared as described in Description 31) and the corresponding amine using an analogous method to that described for Description 47.

| Description | Amine | Mass Spectrum |
|---|---|---|
| 1,1-Dimethylethyl 7-({5- | Dimethylamine | MS(AP+) m/e 311[[M-COOBu^{t}] + H]⁺ |
| [(dimethylamino)carbonyl]-2- | hydrochloride | |
| pyrazinyl}methyl)-1,2,4,5- | | |
| tetrahydro-3H-3-benzazepine- | | |
| 3-carboxylate (D48) | | |
| 1,1-Dimethylethyl 7-[(5-{[(2,2,2- | 2,2,2- | MS(AP+) m/e 365[[M-COOBu^{t}] + H]⁺ |
| trifluoroethyl)amino]carbonyl}- | Trifluoroethylamine | |
| 2-pyrazinyl)methyl]-1,2,4,5- | hydrochloride | |
| tetrahydro-3H-3-benzazepine- | | |
| 3-carboxylate (D49) | | |
| 1,1-Dimethylethyl 7-[(5- | Aminoacetonitrile | MS(AP+) m/e 322[[M-COOBu^{t}] + H]⁺ |
| {[(cyanomethyl)amino]carbonyl}- | bisulfate | |
| 2-pyrazinyl)methyl]-1,2,4,5- | | |
| tetrahydro-3H-3-benzazepine- | | |
| 3-carboxylate (D50) | | |

Description 51

### 1,1-Dimethylethyl 7-{[5-(aminocarbonyl)-2-pyrazinyl]methyl}-1,2,4,5-tetrahydro-3H-3-benzazepine-3-carboxylate (D51)

5-[(3-{[(1,1-Dimethylethyl)oxy]carbonyl}-2,3,4,5-tetrahydro-1H-3-benzazepin-7-yl)methyl]-2-pyrazinecarboxylic acid (may be prepared as described in Description 31) (84 mg, 0.22 mmol) was dissolved in dichloromethane (4.5 ml), treated with N,N′-carbonyldiimidazole (71 mg, 0.44 mmol) and stirred at room temperature for 18 hours. The reaction mixture was treated with 0.88 ammonia solution (0.05 ml, 0.88 mmol) and the resulting mixture stirred for 2 hours. The reaction mixture was applied directly to a silica column and the product purified by column chromatography eluting with ethyl acetate:pentane (1:1) to afford the title product. MS (AP+) m/e 281 [[M-COOBu^{t}]+H]⁺.

Descriptions 52-53 (D52-53)

The following intermediates may be prepared from 5-[(3-{[(1,1-dimethylethyl)oxy]carbonyl}-2,3,4,5-tetrahydro-1H-3-benzazepin-7-yl)methyl]-2-pyridinecarboxylic acid (may be prepared as described in Description 22) and the corresponding amine using an analogous method to that described in Description 51.

| Description | Amine | Mass Spectrum |
|---|---|---|
| 1,1-Dimethylethyl 7-({6- | Methylamine* | MS(AP+) m/e |
| [(methylamino)carbonyl]-3- | | 296[[M-COOBu^{t}] + H]⁺ |
| pyridinyl}methyl)-1,2,4,5- | | |
| tetrahydro-3H-3- | | |
| benzazepine-3-carboxylate | | |
| (D52) | | |
| 1,1-Dimethylethyl 7-{[6- | Ammonia | MS(AP+) m/e |
| (aminocarbonyl)-3- | | 282[[M-COOBu^{t}] + H]⁺ |
| pyridinyl]methyl}-1,2,4,5- | | |
| tetrahydro-3H-3- | | |
| benzazepine-3-carboxylate | | |
| (D53) | | |

*Amine used was 2M methylamine in THF.

Description 54

### 1,1-Dimethylethyl 7-[(4-bromophenyl)methyl]-1,2,4,5-tetrahydro-3H-3-benzazepine-3-carboxylate (D54)

(3-{[(1,1-Dimethylethyl)oxy]carbonyl}-2,3,4,5-tetrahydro-1H-3-benzazepin-7-yl)boronic acid (may be prepared by the method described in WO 2004056369) (200 mg, 0.69 mmol) and 4-bromobenzyl bromide (181 mg, 0.72 mmol) were dissolved in a 2:3 mixture of ethanol and toluene (5 ml) and treated with 1M aqueous sodium carbonate solution (0.83 ml, 0.83 mmol) and tetrakis(triphenylphosphine)palladium (0) (24 mg, 0.021 mmol). The resulting mixture was stirred at room temperature under argon for 1 hour and heated under reflux for 4 hours under argon. The reaction was cooled to room temperature and left to stand overnight at room temperature. The reaction was diluted with ethyl acetate and filtered through celite. The filtrate washed with water, separated, dried over magnesium sulphate and concentrated in vacuo. The resulting residue was purified by column chromatography eluting with ethyl acetate:pentane (1:9) to afford the title product. MS (AP+) m/e 317 [[M-COOBu^{t}]+H]⁺.

Description 55

### 1,1-Dimethylethyl 7-{[4-(2-oxo-1-pyrrolidinyl)phenyl]methyl}-1,2,4,5-tetrahydro-3H-3-benzazepine-3-carboxylate (D55)

1,1-Dimethylethyl 7-[(4-bromophenyl)methyl]-1,2,4,5-tetrahydro-3H-3-benzazepine-3-carboxylate (may be prepared as described in Description 54) (174 mg, 0.42 mmol), 2-pyrrolidinone (0.06 ml, 0.84 mmol), potassium carbonate (209 mg, 1.51 mmol), copper (I) iodide (24 mg, 0.13 mmol) and dimethyl-1,2-ethanediamine (0.01 ml, 0.13 mmol) were added together in dry dioxan (4 ml) and the resulting mixture heated under reflux under argon for 18 hours. The reaction mixture was allowed to cool to room temperature, diluted with water and extracted with ethyl acetate (×2). The ethyl acetate layers were separated, combined, dried under magnesium sulphate and evaporated in vacuo. The resulting residue was purified by column chromatography eluting with ethyl acetate:pentane (1:1) to afford the title product. MS (AP+) m/e 321 [[M-COOBu^{t}]+H]⁺.

Description 56

### 1-(5-Methyl-2-pyridinyl)-2-pyrrolidinone (D56)

The title compound was prepared from 2-bromo-5-methylpyridine and 2-pyrrolidinone using an analogous method to that described in Description 55. MS (AP+) m/e 177 [M+H]⁺.

Description 57

### 1-[5-(Bromomethyl)-2-pyridinyl]-2-pyrrolidinone (D57)

To a solution of 1-(5-Methyl-2-pyridinyl)-2-pyrrolidinone (may be prepared as described in Description 56) (0.85 g, 4.82 mmol) in carbon tetrachloride (10 ml) was added N-bromosuccinimide (0.86 g, 4.83 mmol) and 2,2′-azobis(2-methylpropionitrile) (48 mg, 0.29 mmol). The resulting mixture was heated under reflux for 3 hours, allowed to cool to room temperature and filtered. The solid was discarded and the filtrate was evaporated in vacuo. The resulting residue was purified by column chromatography eluting with ethyl acetate:dichloromethane (1:9) to afford the title product. MS (AP+) m/e 257 [M+2H]⁺.

Description 58

### 1,1-Dimethylethyl 7-{[6-(2-oxo-1-pyrrolidinyl)-3-pyridinyl]methyl}-1,2,4,5-tetrahydro-3H-3-benzazepine-3-carboxylate (D58)

The title compound was prepared from (3-{[(1,1-dimethylethyl)oxy]carbonyl}-2,3,4,5-tetrahydro-1H-3-benzazepin-7-yl)boronic acid (may be prepared by the method described in WO 2004056369) and 1-[5-(bromomethyl)-2-pyridinyl]-2-pyrrolidinone (may be prepared as described in Description 57) using an analogous method to that described in Description 22. MS (AP+) m/e 422 [M+H]⁺.

Description 59

### 1,1-Dimethylethyl 7-(2-pyrazinylmethyl)-1,2,4,5-tetrahydro-3H-3-benzazepine-3-carboxylate (1)₅₉)

A mixture of 1,1-dimethylethyl 7-(bromomethyl)-1,2,4,5-tetrahydro-3H-3-benzazepine-3-carboxylate (100 mg, 0.29 mmol) (may be prepared as described in Description 24), 2-tributylstannylpyrazine (161 mg, 0.44 mmol), tetrakis(triphenylphosphine)palladium (0) (17 mg, 0.015 mmol) and lithium chloride (37 mg, 0.87 mmol) in toluene (3 ml) was heated under reflux under argon for 3 hours. The reaction mixture was allowed to cool to room temperature, filtered through celite and the solvent removed in vacuo. The resulting residue was purified by column chromatography eluting with ethyl acetate:pentane (1:4 to 1:1) to afford the title product. MS (AP+) m/e 240 [[M-COOBu^{t}]+H]⁺.

Description 60

### 1,1-Dimethylethyl 7-[(5-amino-2-pyrazinyl)methyl]-1,2,4,5-tetrahydro-3H-3-benzazepine-3-carboxylate (D60)

A mixture of 1,1-dimethylethyl 7-(bromomethyl)-1,2,4,5-tetrahydro-3H-3-benzazepine-3-carboxylate (346 mg, 1.02 mmol) (may be prepared as described in Description 24), 5-(trimethylstannanyl)-2-pyrazinamine (may be prepared by the method described in Chemistry: A European Journal, 2000, 6, 22, 4132-4139) (394 mg, 1.53 mmol), bis(triphenylphosphine)palladium (II) chloride (36 mg, 0.05 mmol) in dioxane (7 ml) was heated under reflux under argon for 2 hours. The reaction mixture was allowed to cool to room temperature and the solvent removed in vacuo. The resulting residue was purified by column chromatography eluting with ethyl acetate:pentane (1:1 to 4:1) to afford the title product. MS (AP+) m/e 355 [M+H]⁺.

Description 61

### 1,1-Dimethylethyl 7-({5-[(4-bromobutanoyl)amino]-2-pyrazinyl}methyl)-1,2,4,5-tetrahydro-3H-3-benzazepine-3-carboxylate (D61)

1,1-Dimethylethyl 7-[(5-amino-2-pyrazinyl)methyl]-1,2,4,5-tetrahydro-3H-3-benzazepine-3-carboxylate (may be prepared as described in Description 60) (150 mg, 0.42 mmol) was dissolved in dichloromethane (4 ml), treated with pyridine (0.04 ml, 0.46 mmol) and cooled to 0° C. under argon. A solution of 4-bromobutyrl chloride (0.05 ml, 0.46 mmol) in dichloromethane (2 ml) was added drop wise and the resulting mixture stirred at 0° C. for 10 minutes. The reaction mixture was allowed to warm to room temperature, stirred for 18 hours, diluted with dichloromethane and washed with saturated sodium bicarbonate solution. The dichloromethane layer was separated, dried under magnesium sulfate and evaporated in vacuo. The resulting residue was purified by column chromatography eluting with ethyl acetate:pentane (1:1) to afford the title product. MS (AP+) m/e 448 [[M-Bu^{t}]+H]⁺.

Description 62

### 1,1-Dimethylethyl 7-{[5-(2-oxo-1-pyrrolidinyl)-2-pyrazinyl]methyl}-1,2,4,5-tetrahydro-3H-3-benzazepine-3-carboxylate (D62)

1,1-Dimethylethyl 7-({5-[(4-bromobutanoyl)amino]-2-pyrazinyl}methyl)-1,2,4,5-tetrahydro-3H-3-benzazepine-3-carboxylate (may be prepared as described in Description 61) (106 mg, 0.21 mmol) was dissolved in N,N-dimethylformamide (3 ml), cooled in an ice bath and treated with sodium hydride (60% in mineral oil, 9 mg, 0.23 mmol). The resulting mixture was stirred for 10 minutes and then allowed to warm to room temperature and stirred for 2.5 hours. The reaction was cooled in an ice bath, treated with water (5 ml), allowed to warm to room temperature and extracted with ethyl acetate (×3). The ethyl acetate layers were combined, dried under magnesium sulphate and evaporated in vacuo. The resulting residue was purified by column chromatography eluting with ethyl acetate:pentane (1:1) to afford the title product. MS (AP+) m/e 323 [[M-COOBu^{t}]+H]⁺.

Description 63

### 1,1-Dimethylethyl 7-{[5-(acetylamino)-2-pyrazinyl]methyl}-1,2,4,5-tetrahydro-3H-3-benzazepine-3-carboxylate (D63)

The title compound may be prepared from 1,1-dimethylethyl 7-[(5-amino-2-pyrazinyl)methyl]-1,2,4,5-tetrahydro-3H-3-benzazepine-3-carboxylate (may be prepared as described in Description 60) and acetyl chloride using an analogous method to that described in Description 61. MS (AP+) m/e 395 [M−H]⁺.

Description 64

### N-Methyl-5-(2,3,4,5-tetrahydro-1H-3-benzazepin-7-ylmethyl)-2-pyrazinecarboxamide (D64)

1,1-dimethylethyl 7-({5-[(methylamino)carbonyl]-2-pyrazinyl}methyl)-1,2,4,5-tetrahydro-3H-3-benzazepine-3-carboxylate (may be prepared as described in Description 44) (84 mg, 0.21 mmol) was dissolved in dichloromethane (1.5 ml), treated with trifluoroacetic acid (1.5 ml) and the resulting mixture was stirred for 30 minutes. The solvent was evaporated and the residue dissolved in methanol and applied to an SCX column, eluting with methanol and 2M ammonia/methanol. The basic fractions were combined and evaporated to afford the title product. MS (AP+) m/e 297 [M+H]⁺.

Description 65

### N-Ethyl-5-(2,3,4,5-tetrahydro-1H-3-benzazepin-7-ylmethyl)-2-pyrazinecarboxamide (D65)

To a solution of 1,1-dimethylethyl 7-({5-[(ethylamino)carbonyl]-2-pyrazinyl}methyl)-1,2,4,5-tetrahydro-3H-3-benzazepine-3-carboxylate (may be prepared as described in Description 47) (56 mg, 0.14 mmol) in dichloromethane (1 ml) was added trifluoroacetic acid (1 ml) and the resulting mixture was stirred for 30 minutes. The solvent was evaporated, the residue was dissolved in methanol, applied to an SCX ion exchange cartridge and eluted with methanol and 2M ammonia/methanol. The basic fractions were combined and evaporated to afford the title product. MS (AP+) m/e 311 [M+H]⁺.

Descriptions 66-85 (D66-85)

The following intermediates may be prepared from the corresponding benzazepine starting material using an analogous method to that described in Description 20:

| | Benzazepine Starting | |
|---|---|---|
| Description | Material | Mass Spectrum |
| 5-(2,3,4,5-Tetrahydro- | 1,1-Dimethylethyl 7-[(6- | MS(AP+) m/e 264[M + H]⁺ |
| 1H-3-benzazepin-7- | cyano-3-pyridinyl)methyl]- | |
| ylmethyl)-2- | 1,2,4,5-tetrahydro-3H-3- | |
| pyridinecarbonitrile | benzazepine-3-carboxylate | |
| (D66) | (may be prepared as | |
| | described in Description 21) | |
| 7-{[6-(3-Methyl-1,2,4- | 1,1-Dimethylethyl 7-{[6-(3- | MS(AP+) m/e 321[M + H]⁺ |
| oxadiazol-5-yl)-3- | methyl-1,2,4-oxadiazol-5-yl)- | |
| pyridinyl]methyl}- | 3-pyridinyl]methyl}-1,2,4,5- | |
| 2,3,4,5-tetrahydro-1H- | tetrahydro-3H-3- | |
| 3-benzazepine (D67) | benzazepine-3-carboxylate | |
| | (may be prepared as | |
| | described in Description 23) | |
| 7-{[5-(3-Methyl-1,2,4- | 1,1-Dimethylethyl 7-{[5-(3- | MS(AP+), m/e 322[M + H]⁺ |
| oxadiazol-5-yl)-2- | methyl-1,2,4-oxadiazol-5-yl)- | |
| pyrazinyl]methyl}- | 2-pyrazinyl]methyl}-1,2,4,5- | |
| 2,3,4,5-tetrahydro-1H- | tetrahydro-3H-3- | |
| 3-benzazepine (D68) | benzazepine-3-carboxylate | |
| | (may be prepared as | |
| | described in Description 32) | |
| 7-{[5-(3-Methyl-1,2,4- | 1,1-Dimethylethyl 7-{[5-(3- | MS(AP+), m/e 321[M + H]⁺ |
| oxadiazol-5-yl)-2- | methyl-1,2,4-oxadiazol-5-yl)- | |
| pyridinyl]methyl}- | 2-pyridinyl]methyl}-1,2,4,5- | |
| 2,3,4,5-tetrahydro-1H- | tetrahydro-3H-3- | |
| 3-benzazepine (D69) | benzazepine-3-carboxylate | |
| | (may be prepared as | |
| | described in Description 40) | |
| N,N-Dimethyl-4- | 1,1-Dimethylethyl 7-({4- | MS(AP+), m/e 309[M + H]⁺ |
| (2,3,4,5-tetrahydro-1H- | [(dimethylamino)carbonyl]phenyl} | |
| 3-benzazepin-7- | methyl)-1,2,4,5- | |
| ylmethyl)benzamide | tetrahydro-3H-3- | |
| (D70) | benzazepine-3-carboxylate | |
| | (may be prepared as | |
| | described in Description 41) | |
| 7-{[4-(1- | 1,1-Dimethylethyl 7-{[4-(1- | MS(AP+), m/e 349[M + H]⁺ |
| Piperidinylcarbonyl)phenyl] | piperidinylcarbonyl)phenyl]methyl}- | |
| methyl}-2,3,4,5- | 1,2,4,5-tetrahydro-3H- | |
| tetrahydro-1H-3- | 3-benzazepine-3-carboxylate | |
| benzazepine (D71) | (may be prepared as | |
| | described in Description 42) | |
| 7-{[4-(4- | 1,1-Dimethylethyl 7-{[4-(4- | MS(AP+), m/e 351[M + H]⁺ |
| Morpholinylcarbonyl)phenyl] | morpholinylcarbonyl)phenyl] | |
| methyl}-2,3,4,5- | methyl}-1,2,4,5-tetrahydro- | |
| tetrahydro-1H-3- | 3H-3-benzazepine-3- | |
| benzazepine (D72) | carboxylate (may be | |
| | prepared as described in | |
| | Description 43) | |
| 7-{[5-(1- | 1,1-Dimethylethyl 7-{[5-(1- | MS(AP+), m/e 337[M + H]⁺ |
| Pyrrolidinylcarbonyl)- | pyrrolidinylcarbonyl)-2- | |
| 2-pyrazinyl]methyl}- | pyrazinyl]methyl}-1,2,4,5- | |
| 2,3,4,5-tetrahydro-1H- | tetrahydro-3H-3- | |
| 3-benzazepine (D73) | benzazepine-3-carboxylate | |
| | (may be prepared as | |
| | described in Description 45) | |
| N-(1-Methylethyl)-5- | 1,1-Dimethylethyl 7-[(5-{[(1- | MS(AP+), m/e 325[M + H]⁺ |
| (2,3,4,5-tetrahydro-1H- | methylethyl)amino]carbonyl}- | |
| 3-benzazepin-7- | 2-pyrazinyl)methyl]-1,2,4,5- | |
| ylmethyl)-2- | tetrahydro-3H-3- | |
| pyrazinecarboxamide | benzazepine-3-carboxylate | |
| (D74) | (may be prepared as | |
| | described in Description 46) | |
| N,N-Dimethyl-5- | 1,1-Dimethylethyl 7-({5- | MS(AP+), m/e 311[M + H]⁺ |
| (2,3,4,5-tetrahydro-1H- | [(dimethylamino)carbonyl]-2- | |
| 3-benzazepin-7- | pyrazinyl}methyl)-1,2,4,5- | |
| ylmethyl)-2- | tetrahydro-3H-3- | |
| pyrazinecarboxamide | benzazepine-3-carboxylate | |
| (D75) | (may be prepared as | |
| | described in Description 48) | |
| 5-(2,3,4,5-Tetrahydro- | 1,1-Dimethylethyl 7-[(5- | MS(AP+), m/e 365[M + H]⁺ |
| 1H-3-benzazepin-7- | {[(2,2,2- | |
| ylmethyl)-N-(2,2,2- | trifluoroethyl)amino]carbonyl}- | |
| trifluoroethyl)-2- | 2-pyrazinyl)methyl]-1,2,4,5- | |
| pyrazinecarboxamide | tetrahydro-3H-3- | |
| (D76) | benzazepine-3-carboxylate | |
| | (may be prepared as | |
| | described in Description 49) | |
| N-(Cyanomethyl)-5- | 1,1-Dimethylethyl 7-[(5- | MS(AP+), m/e 322[M + H]⁺ |
| (2,3,4,5-tetrahydro-1H- | {[(cyanomethyl)amino]carbonyl}- | |
| 3-benzazepin-7- | 2-pyrazinyl)methyl]- | |
| ylmethyl)-2- | 1,2,4,5-tetrahydro-3H-3- | |
| pyrazinecarboxamide | benzazepine-3-carboxylate | |
| (D77) | (may be prepared as | |
| | described in Description 50) | |
| 5-(2,3,4,5-Tetrahydro- | 1,1-Dimethylethyl 7-{[5- | MS(AP+), m/e 283[M + H]⁺ |
| 1H-3-benzazepin-7- | (aminocarbonyl)-2- | |
| ylmethyl)-2- | pyrazinyl]methyl}-1,2,4,5- | |
| pyrazinecarboxamide | tetrahydro-3H-3- | |
| (D78) | benzazepine-3-carboxylate | |
| | (may be prepared as | |
| | described in Description 51) | |
| N-Methyl-5-(2,3,4,5- | 1,1-Dimethylethyl 7-({6- | MS(AP+), m/e 296[M + H]⁺ |
| tetrahydro-1H-3- | [(methylamino)carbonyl]-3- | |
| benzazepin-7- | pyridinyl}methyl)-1,2,4,5- | |
| ylmethyl)-2- | tetrahydro-3H-3- | |
| pyridinecarboxamide | benzazepine-3-carboxylate | |
| (D79) | (may be prepared as | |
| | described in Description 52) | |
| 5-(2,3,4,5-Tetrahydro- | 1,1-Dimethylethyl 7-{[6- | MS(AP+), m/e 282[M + H]⁺ |
| 1H-3-benzazepin-7- | (aminocarbonyl)-3- | |
| ylmethyl)-2- | pyridinyl]methyl}-1,2,4,5- | |
| pyridinecarboxamide | tetrahydro-3H-3- | |
| (D80) | benzazepine-3-carboxylate | |
| | (may be prepared as | |
| | described in Description 53) | |
| 1-[4-(2,3,4,5- | 1,1-Dimethylethyl 7-{[4-(2- | MS(AP+), m/e 321[M + H]⁺ |
| Tetrahydro-1H-3- | oxo-1- | |
| benzazepin-7- | pyrrolidinyl)phenyl]methyl}- | |
| ylmethyl)phenyl]-2- | 1,2,4,5-tetrahydro-3H-3- | |
| pyrrolidinone (D81) | benzazepine-3-carboxylate | |
| | (may be prepared as | |
| | described in Description 55) | |
| 1-[5-(2,3,4,5- | 1,1-Dimethylethyl 7-{[6-(2- | MS(AP+), m/e 322[M + H]⁺ |
| Tetrahydro-1H-3- | oxo-1-pyrrolidinyl)-3- | |
| benzazepin-7- | pyridinyl]methyl}-1,2,4,5- | |
| ylmethyl)-2-pyridinyl]- | tetrahydro-3H-3- | |
| 2-pyrrolidinone (D82) | benzazepine-3-carboxylate | |
| | (may be prepared as | |
| | described in Description 58) | |
| 7-(2-Pyrazinylmethyl)- | 1,1-Dimethylethyl 7-(2- | MS(AP+), m/e 240[M + H]⁺ |
| 2,3,4,5-tetrahydro-1H- | pyrazinylmethyl)-1,2,4,5- | |
| 3-benzazepine (D83) | tetrahydro-3H-3- | |
| | benzazepine-3-carboxylate | |
| | (may be prepared as | |
| | described in Description 59) | |
| 1-[5-(2,3,4,5- | 1,1-Dimethylethyl 7-{[5-(2- | MS(AP+), m/e 323[M + H]⁺ |
| Tetrahydro-1H-3- | oxo-1-pyrrolidinyl)-2- | |
| benzazepin-7- | pyrazinyl]methyl}-1,2,4,5- | |
| ylmethyl)-2-pyrazinyl]- | tetrahydro-3H-3- | |
| 2-pyrrolidinone (D84) | benzazepine-3-carboxylate | |
| | (may be prepared as | |
| | described in Description 62) | |
| N-[5-(2,3,4,5- | 1,1-Dimethylethyl 7-{[5- | MS(AP+), m/e 297[M + H]⁺ |
| Tetrahydro-1H-3- | (acetylamino)-2- | |
| benzazepin-7- | pyrazinyl]methyl}-1,2,4,5- | |
| ylmethyl)-2- | tetrahydro-3H-3- | |
| pyrazinyl]acetamide | benzazepine-3-carboxylate | |
| (D85) | (may be prepared as | |
| | described in Description 63) | |

Description 86

### 1,1-Dimethylethyl 7-[(6-bromo-3-pyridinyl)methyl]-1,2,4,5-tetrahydro-3H-3-benzazepine-3-carboxylate (D86)

The title compound was prepared from (3-{[(1,1-dimethylethyl)oxy]carbonyl}-2,3,4,5-tetrahydro-1H-3-benzazepin-7-yl)boronic acid (may be prepared using the method described in WO 2004056369) and 2-bromo-5-(bromomethyl)pyridine (may be prepared using the method described in WO 2005016876) using an analogous method to that described in Description 21. MS (AP+) m/e 318 [[M-COOBu^{t}]+H]⁺.

Description 87

### 7-[(6-Bromo-3-pyridinyl)methyl]-2,3,4,5-tetrahydro-1H-3-benzazepine (D87)

The title compound was prepared from 1,1-dimethylethyl 7-[(6-bromo-3-pyridinyl)methyl]-1,2,4,5-tetrahydro-3H-3-benzazepine-3-carboxylate (may be prepared as described in Description 86) using an analogous method to that described in Description 19. MS (AP+) m/e 319 [M+2H]⁺.

Description 88

### N-methyl-6-(2,3,4,5-tetrahydro-1H-3-benzazepin-7-ylmethyl)-3-pyridinecarboxamide (D88)

Trifluoroacetic acid (3 ml) was added to a solution of 1,1-dimethylethyl 7-({5-[(methylamino)carbonyl]-2-pyridinyl}methyl)-1,2,4,5-tetrahydro-3H-3-benzazepine-3-carboxylate (may be prepared as described in Description 37) (109 mg, 0.28 mmol) in dichloromethane (5 ml) at 0° C. under argon. The mixture was stirred for 15 minutes and then applied to a SCX cartridge (Varian bond-elute, 10 g) and washed with methanol followed by a mixture of 2M ammonia/methanol. The basic fractions were combined, evaporated to afford the title compound. MS (ES+) m/e 296 [M+H]⁺.

Descriptions 89-90

Intermediates 89-90 may be prepared from appropriate amides (D38-39) using an analogous method to that described for Description 88 (see table)

| | | LC/MS |
|---|---|---|
| Description | Amide | (M + H⁺) |
| 7-{[5-(1-pyrrolidinylcarbonyl)-2- | 1,1-dimethylethyl 7-{[5-(1- | 336 |
| pyridinyl]methyl}-2,3,4,5- | pyrrolidinylcarbonyl)-2- | |
| tetrahydro-1H-3-benzazepine | pyridinyl]methyl}-1,2,4,5-tetrahydro- | |
| (D89) | 3H-3-benzazepine-3-carboxylate (may | |
| | be prepared as described in | |
| | Description 38) | |
| 6-(2,3,4,5-tetrahydro-1H-3- | 1,1-dimethylethyl 7-{[5- | 282 |
| benzazepin-7-ylmethyl)-3- | (aminocarbonyl)-2-pyridinyl]methyl}- | |
| pyridinecarboxamide (D90) | 1,2,4,5-tetrahydro-3H-3-benzazepine- | |
| | 3-carboxylate (may be prepared as | |
| | described in Description 39) | |

Description 91

### 1,1-dimethylethyl 7-{[4-(1,2,3-thiadiazol-4-yl)phenyl]methyl}-1,2,4,5-tetrahydro-3H-3-benzazepine-3-carboxylate (D91)

To a solution of 4-[4-(bromomethyl)phenyl]-1,2,3-thiadiazole (180 mg, 0.70 mml) in dimethoxyethane (2 ml) was added sequentially, tetrakis(triphenylphosphine)palladium(0) (41.0 mg, 0.04 mmol), a solution of (3-{[(1,1-dimethylethyl)oxy]carbonyl}-2,3,4,5-tetrahydro-1H-3-benzazepin-7-yl)boronic acid (may be prepared using the method described in WO2004056369) (306 mg, 1.05 mmol) in dimethoxyethane/ethanol (1:1, 2 ml), and a 2M aqueous sodium carbonate solution (0.9 ml, 1.75 mmol). The resulting mixture was heated at reflux, under argon, for 24 hours. Diluted with ethyl acetate (10 ml) and water (10 ml) and filtered through celite, washing through with ethyl acetate. The ethyl acetate layer was washed with water and brine, dried over sodium sulphate and evaporated to dryness. The product was purified by chromatography on silica, eluting with a mixture of ethyl acetate and pentane (0-60%) to afford the title compound. MS (ES+) m/e 322 [M-BOC]⁺.

Descriptions 92-94

Descriptions 92-94 may be prepared from (3-{[(1,1-dimethylethyl)oxy]carbonyl}-2,3,4,5-tetrahydro-1H-3-benzazepin-7-yl)boronic acid (may be prepared using the method described in WO2004056369) and the appropriate benzyl bromides using an analogous method to that described for Description 91 (see table)

| | | LC/MS |
|---|---|---|
| Description | Benzyl bromide | (M-BOC⁺) |
| 1,1-dimethylethyl 7-{[4-(5-oxo-2,5- | 3-[4-(bromomethyl)phenyl]- | 322 |
| dihydro-1,2,4-oxadiazol-3- | 1,2,4-oxadiazol-5(2H)-one | |
| yl)phenyl]methyl}-1,2,4,5-tetrahydro-3H- | | |
| 3-benzazepine-3-carboxylate | | |
| (D92) | | |
| 1,1-dimethylethyl 7-{[4-(1H-pyrazol-1- | 1-[4-(bromomethyl)phenyl]-1H- | 304 |
| yl)phenyl]methyl}-1,2,4,5-tetrahydro- | pyrazole | |
| 3H-3-benzazepine-3-carboxylate | | |
| (D93) | | |
| 1,1-dimethylethyl 7-{[4-(1H-1,2,4- | 1-[4-(bromomethyl)phenyl]-1H- | 305 |
| triazol-1-yl)phenyl]methyl}-1,2,4,5- | 1,2,4-triazole | |
| tetrahydro-3H-3-benzazepine-3- | | |
| carboxylate (D94) | | |

Description 95

### 7-{[4-(1,2,3-thiadiazol-4-yl)phenyl]methyl}-2,3,4,5-tetrahydro-1H-3-benzazepine (D95)

Trifluoroacetic acid (2.5 ml) was added to a solution of 1,1-dimethylethyl 7-{[4-(1,2,3-thiadiazol-4-yl)phenyl]methyl}-1,2,4,5-tetrahydro-3H-3-benzazepine-3-carboxylate (may be prepared as described in Description 91) (220 mg, 0.52 mmol) in dichloromethane (2.5 ml) at 0° C. under argon. The mixture was stirred for 30 minutes and then applied to a SCX cartridge (Varian bond-elute, 5 g) and washed with methanol followed by a mixture of 2M ammonia/methanol. The basic fractions were combined and evaporated to afford the title compound. MS (ES+) m/e 322 [M+H]⁺.

Descriptions 96-98

Descriptions 96-98 may be prepared from the appropriate BOC-benzazepine (D92-D94) and trifluoroacetic acid using an analogous method to that described for Description 95 (see table)

| | | LC/MS |
|---|---|---|
| Description | BOC-benzazepine | (M + H⁺) |
| 3-[4-(2,3,4,5-tetrahydro-1H-3- | 1,1-dimethylethyl 7-{[4-(5-oxo-2,5- | 322 |
| benzazepin-7-ylmethyl)phenyl]- | dihydro-1,2,4-oxadiazol-3- | |
| 1,2,4-oxadiazol-5(2H)-one (D96) | yl)phenyl]methyl}-1,2,4,5-tetrahydro- | |
| | 3H-3-benzazepine-3-carboxylate (may | |
| | be prepared as described in | |
| | Description 92) | |
| 7-{[4-(1H-pyrazol-1- | 1,1-dimethylethyl 7-{[4-(1H-pyrazol-1- | 304 |
| yl)phenyl]methyl}-2,3,4,5- | yl)phenyl]methyl}-1,2,4,5-tetrahydro- | |
| tetrahydro-1H-3-benzazepine | 3H-3-benzazepine-3-carboxylate (may | |
| (D97) | be prepared as described in | |
| | Description 93) | |
| 7-{[4-(1H-1,2,4-triazol-1- | 1,1-dimethylethyl 7-{[4-(1H-1,2,4- | 305 |
| yl)phenyl]methyl}-2,3,4,5- | triazol-1-yl)phenyl]methyl}-1,2,4,5- | |
| tetrahydro-1H-3-benzazepine | tetrahydro-3H-3-benzazepine-3- | |
| (D98) | carboxylate (may be prepared as | |
| | described in Description 94) | |

Description 99

### 1,1-Dimethylethyl 7-({5-[(ethyloxy)carbonyl]-1H-imidazol-1-yl}methyl)-1,2,4,5-tetrahydro-3H-3-benzazepine-3-carboxylate (D99)

To a solution of ethyl 1H-imidazole-4-carboxylate (may be obtained from Combi-Blocks; 0.113 g, 0.81 mmol) in dimethylformamide (3 ml) was added sodium hydride (0.032 g, 0.81 mmol, 60% dispersion in mineral oil). The reaction was stirred at room temperature for 20 minutes then 1,1-dimethylethyl 7-(bromomethyl)-1,2,4,5-tetrahydro-3H-3-benzazepine-3-carboxylate (may be prepared as described in Description 24) (0.250 g, 0.735 mmol) in dimethylformamide (2 ml) was added. The reaction was then heated at 50° C. for 20 minutes before being cooled to room temperature, quenched with water, diluted with brine and extracted with diethyl ether. The ether extracts were then dried, filtered, reduced in vacuo and the resulting crude reaction mixture was purified by column chromatography, eluting with a mixture of ethyl acetate/pentane (4:1) to afford the title product; 0.062 g (21%). (MS (ES+): [M+H]⁺ at m/z 400

Description 100

### 1,1-Dimethylethyl 7-({4-[(ethyloxy)carbonyl]-1H-imidazol-1-yl}methyl)-1,2,4,5-tetrahydro-3H-3-benzazepine-3-carboxylate (D100)

The title compound, 0.120 g (40%) may be isolated from the reaction used to prepare Description 99 using column chromatography, eluting with a mixture of ethyl acetate/pentane (4:1). (MS (ES+): [M+H]⁺ at m/z 400

Description 101

### 1-[(3-{[(1,1-Dimethylethyl)oxy]carbonyl}-2,3,4,5-tetrahydro-1H-3-benzazepin-7-yl)methyl]-1H-imidazole-5-carboxylic acid (D101)

1,1-Dimethylethyl 7-({5-[(ethyloxy)carbonyl]-1H-imidazol-1-yl}methyl)-1,2,4,5-tetrahydro-3H-3-benzazepine-3-carboxylate (may be prepared as described in Description 99) (0.06 g, 0.15 mmol) in methanol was treated with 2M aqueous sodium hydroxide (2 ml) and then stirred at room temperature for 3 hours. The reaction was acidified with acetic acid, extracted into ethyl acetate, dried and reduced in vacuo to furnish the title compound 0.05 g. ¹H NMR (DMSO, d6) 7.90 (1H, s), 7.45 (1H, s), 7.08 (1H, d), 6.99 (1H, s), 6.91 (1H, d), 5.50 (2H,s), 3.41 (m, 4H), 2.77 (4H, m), 1.39 (s, 9H).

Description 102

### 1,1-Dimethylethyl 7-({5-[(methylamino)carbonyl]-1H-imidazol-1-yl}methyl)-1,2,4,5-tetrahydro-3H-3-benzazepine-3-carboxylate (D102)

The title compound was prepared from 1-[(3-{[(1,1-dimethylethyl)oxy]carbonyl}-2,3,4,5-tetrahydro-1H-3-benzazepin-7-yl)methyl]-1H-imidazole-5-carboxylic acid (may be prepared as described in Description 101) and methylamine using an analogous method to that described for Description 37. (MS (ES+): [M+H]⁺ at m/z 385

Description 103

### N-Methyl-1-(2,3,4,5-tetrahydro-1H-3-benzazepin-7-ylmethyl)-1H-imidazole-5-carboxamide (D103)

The title compound was prepared from 1,1-dimethylethyl 7-({5-[(methylamino)carbonyl]-1H-imidazol-1-yl}methyl)-1,2,4,5-tetrahydro-3H-3-benzazepine-3-carboxylate (may be prepared as described in Description 102) using an analogous method to that described for Description 35. MS (ES+): [M+H]⁺ at m/z 285

Description 104

### 1-[(3-{[(1,1-dimethylethyl)oxy]carbonyl}-2,3,4,5-tetrahydro-1H-3-benzazepin-7-yl)methyl]-1H-imidazole-4-carboxylic acid (D104)

The title compound may be prepared from 1,1-dimethylethyl 7-({4-[(ethyloxy)carbonyl]-1H-imidazol-1-yl}methyl)-1,2,4,5-tetrahydro-3H-3-benzazepine-3-carboxylate (may be prepared as described in Description 100) using an analogous method to that described for Description 101. MS (ES+): [M+H]⁺ at m/z 372.

Description 105

### 1,1-Dimethylethyl 7-({4-[(methylamino)carbonyl]-1H-imidazol-1-yl}methyl)-1,2,4,5-tetrahydro-3H-3-benzazepine-3-carboxylate (D105)

The title compound was prepared from 1-[(3-{[(1,1-dimethylethyl)oxy]carbonyl}-2,3,4,5-tetrahydro-1H-3-benzazepin-7-yl)methyl]-1H-imidazole-4-carboxylic acid (may be prepared as described in Description 104) and methylamine using an analogous method to that described for Description 16. MS (ES+): [M+H]⁺ at m/z 385.

Description 106

### N-Methyl-1-(2,3,4,5-tetrahydro-1H-3-benzazepin-7-ylmethyl)-1H-imidazole-4-carboxamide (D106)

The title compound was prepared from 1,1-dimethylethyl 7-({4-[(methylamino)carbonyl]-1H-imidazol-1-yl}methyl)-1,2,4,5-tetrahydro-3H-3-benzazepine-3-carboxylate (may be prepared as described in Description 105) using an analogous method to that described for Description 35. MS (ES+): [M+H]⁺ at m/z 285

### EXAMPLE 1

### 4-[(3-Cyclobutyl-2,3,4,5-tetrahydro-1H-3-benzazepin-7-yl)methyl]benzonitrile (E1)

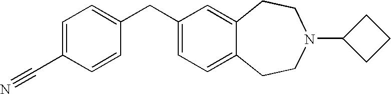

To a solution of 4-(2,3,4,5-Tetrahydro-1H-3-benzazepin-7-ylmethyl)benzonitrile (may be prepared as described in Description 2) (99 mg, 0.34 mmol) in a mixture of dichloromethane: acetic acid (99:1) was added cyclobutanone (36 mg, 0.51 mmol) and the reaction stirred for 1 hour at room temperature. The reaction was then treated with sodium triacetoxyborohydride (109 mg, 0.51 mmol) and stirred for a further 3 hours at room temperature. Methanol (1 ml) was added and the reaction stirred for 15 minutes. The reaction mixture was then applied to an SCX ion exchange cartridge (Varian bond-elute, 5 g) and washed with methanol then water, and then a 2M solution of ammonia in methanol. The combined basic fractions were concentrated in vacuo to afford the title product. MS (AP+) m/e 317 [M+H]⁺.

### EXAMPLE 2

### 5-{[(3-Cyclobutyl-2,3,4,5-tetrahydro-1H-3-benzazepin-7-yl)methyl]oxy}-N-methyl-2-pyrazinecarboxamide (E2)

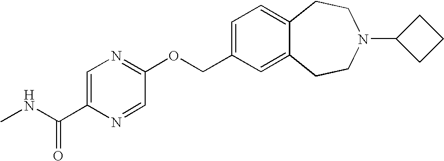

Sodium triacetoxyborohydride (68 mg; 0.32 mmol) was added to a stirring mixture of N-methyl-5-[(2,3,4,5-tetrahydro-1H-3-benzazepin-7-ylmethyl)oxy]-2-pyrazinecarboxamide (may be prepared as described in Description 9) (50 mg; 0.16 mmol), cyclobutanone (30 μl; 0.32 mmol) and glacial acetic acid in dichloromethane (5 ml) and the mixture stirred at room temperature for 3 days. The mixture was diluted with methanol and purified on an SCX ion exchange cartridge eluting with methanol and then 2M ammonia in methanol. Fractions containing the product were combined and evaporated, and the residue purified on silica gel chromatography eluting with a mixture 2M ammonia in methanol solution:dichloromethane (3:97) to afford the title product (42 mg; 64%); MS (AP+) m/e 367 [M+H]⁺.

### EXAMPLE 3

### 5-{[(3-Cyclopentyl-2,3,4,5-tetrahydro-1H-3-benzazepin-7-yl)methyl]oxy}-N-methyl-2-pyrazinecarboxamide (E3)

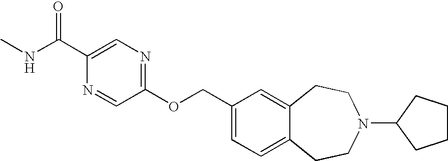

Example 3 (E3) was prepared from N-methyl-5-[(2,3,4,5-tetrahydro-1H-3-benzazepin-7-ylmethyl)oxy]-2-pyrazinecarboxamide (may be prepared as described in Description 9) in an analogous manner to Example 2, substituting cyclopentanone for cyclobutanone; (34 mg; 56%) MS (AP+) m/e 381 [M+H]⁺.

### EXAMPLE 4

### 7-[(5-bromo-2-pyridinyl)methyl]-3-cyclobutyl-2,3,4,5-tetrahydro-1H-3-benzazepine (E4)

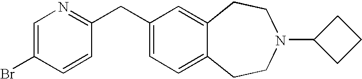

(5-bromo-2-pyridinyl)(3-cyclobutyl-2,3,4,5-tetrahydro-1H-3-benzazepin-7-yl)acetonitrile (may be prepared as described in Description 28) (180 mg, 0.45 mmol) was refluxed in 48% aqueous hydrogen bromide for 4 hours. The cooled reaction mixture was then added to a stirred aqueous solution of potassium carbonate (45 wt %) (9 ml). The pH was adjusted to 14 by addition of 12.5M aqueous sodium hydroxide and extracted with ethyl acetate, washed with water then brine and dried over sodium sulphate and evaporated in vacuo to afford the title product 25 mg, (15%). [M+H]+at m/z 371/373 (C₂₀H₂₃BrN₂ requires [M+H]⁺ at m/z 3371/373).

### EXAMPLE 5

### 1-{6-[(3-cyclobutyl-2,3,4,5-tetrahydro-1H-3-benzazepin-7-yl)methyl]-3-pyridinyl}-2-pyrrolidinone (E5)

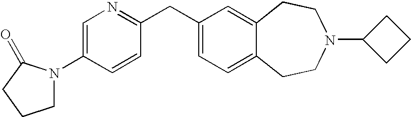

To a solution of 7-[(5-bromo-2-pyridinyl)methyl]-3-cyclobutyl-2,3,4,5-tetrahydro-1H-3-benzazepine (may be prepared as described in Example 4) (25 mg, 0.067 mmol), in 1,4-dioxan (2 ml) was added sequentially 2-pyrrolidinone (12 mg, 0.135 mmol), potassium carbonate (33 mg, 0.242 mmol), N,N′-dimethylethylenediamine (9 mg, 0.0067 mmol) and copper I iodide (2 mg, 0.0067 mmol) and the reaction heated at reflux for 4 hours. The solvent was evaporated in vacuo and the residue partitioned between ethyl acetate and water. The organic phase was separated, washed with water, brine and dried over sodium sulphate and evaporated in vacuo to a crude residue which was purified using silica gel chromatography eluting with a mixture of 2M ammonia in methanol/dichloromethane to afford the title product 10 mg, (40%). (MS (ES+): [M+H]⁺ at m/z 376 (C₂₄H₂₉BrN₃₀ requires [M+H]⁺ at m/z 376).

### EXAMPLE 6

### 6-{[(3-Cyclobutyl-2,3,4,5-tetrahydro-1H-3-benzazepin-7-yl)methyl]oxy}-N-methyl-3-pyridinecarboxamide (E6)

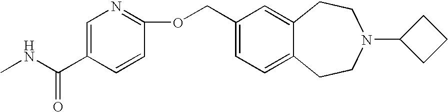

Cyclobutylketone (30 μl, 0.44 mmol) and glacial acetic acid (0.5 ml) were added to a stirring solution of N-methyl-6-[(2,3,4,5-tetrahydro-1H-3-benzazepin-7-ylmethyl)oxy]-3-pyridinecarboxamide (may be prepared as described in Description 13) (70 mg, 0.22 mmol) in dichloromethane (5 ml) cooled to 0° C. under argon. After 30 minutes sodium triacetoxyborohydride (93 mg, 0.44 mmol) was added and the mixture stirred for 3 hours whilst warming to room temperature. The mixture was diluted with methanol and purified on a Bondelut SCX ion exchange cartridge eluting with methanol and then 2M ammonia in methanol. The basic fractions were combined and evaporated. The residue was purified by column chromatography on silica eluting with 95-5 dichloromethane-2M ammonia in methanol to afford the title compound. MS (AP+) m/e 366 [M+H]⁺.

### EXAMPLES 7-8

### E7-8

Examples 7 and 8 (E7 & E8) may be prepared from N-methyl-6-[(2,3,4,5-tetrahydro-1H-3-benzazepin-7-ylmethyl)oxy]-3-pyridinecarboxamide (may be prepared as described in Description 13) and the appropriate carbonyl compound using an analogous method to that used to prepare example 6:

| | Carbonyl | |
|---|---|---|
| Example | Compound | MS (AP+) |
| 6-{[(3-Cyclopentyl- | Cyclopentanone | m/e |
| 2,3,4,5-tetrahydro- | | |
| 1H-3-benzazepin-7-yl)methyl]oxy}- | | 380[M + H]⁺. |
| N-methyl-3-pyridinecarboxamide | | |
| (E7) | | |
| N-Methyl-6-({[3-(2-methylpropyl)- | 2-Methylpropanal | m/e |
| 2,3,4,5-tetrahydro-1H-3-benzazepin- | | 368[M + H]⁺. |
| 7-yl]methyl}oxy)-3- | | |
| pyridinecarboxamide (E8) | | |

### EXAMPLE 9

### 6-{[(3-Cyclobutyl-2,3,4,5-tetrahydro-1H-3-benzazepin-7-yl)methyl]oxy}-N-methyl-3-pyridazinecarboxamide (E9)

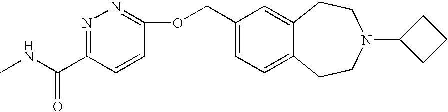

N-methyl-6-[(2,3,4,5-tetrahydro-1H-3-benzazepin-7-ylmethyl)oxy]-3-pyridazinecarboxamide (may be prepared as described in Description 17) (70 mg, 0.22 mmol) was dissolved in dry dichloromethane (5 ml) and cooled in an ice bath to 0° C. This was then treated with cyclobutanone (30 μl, 0.44 mmol) and glacial acetic acid (0.5 ml) and stirred for 30 minutes under argon. Sodium triacetoxyborohydride (93 mg, 0.44 mmol) was added portionwise and the mixture stirred for 3 hours at room temperature under argon. The mixture was diluted with methanol and purified on an SCX ion exchange cartridge eluting with methanol and then 2M ammonia in methanol. The basic fractions were combined and evaporated. The residue was purified on flash silica eluting with 5% 2M ammonia/methanol in dichloromethane. The solvent was evaporated to afford the title compound as a white solid. MS (AP+) m/e 367 [M+H]⁺.

### EXAMPLES 10-11

### E10-11

Examples 10 and 11 (E10 & E11) may be prepared from 6-{[(3-Cyclobutyl-2,3,4,5-tetrahydro-1H-3-benzazepin-7-yl)methyl]oxy}-N-methyl-3-pyridazine carboxamide (may be prepared as described in Description 17) and the appropriate carbonyl compound by an analogous method to that used to prepare example 9:

| | Carbonyl | |
|---|---|---|
| Example | Compound | MS (AP+) |
| 6-{[(3-Cyclopentyl-2,3,4,5-tetrahydro- | Cyclopentanone | m/e |
| 1H-3-benzazepin-7-yl)methyl]oxy}-N- | | 381[M + |
| methyl-3-pyridazinecarboxamide (E10) | | H]⁺. |
| N-Methyl-6-({[3-(2-methylpropyl)- | 2-Methylpropanal | m/e |
| 2,3,4,5-tetrahydro-1H-3-benzazepin-7- | | 369[M + |
| yl]methyl}oxy)-3-pyridazine | | H]⁺. |
| carboxamide (E11) | | |

### EXAMPLE 12

### 5-[(3-Cyclobutyl-2,3,4,5-tetrahydro-1H-3-benzazepin-7-yl)methyl]-2-pyridinecarbonitrile (E12)

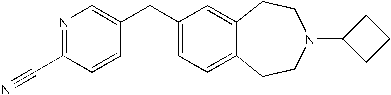

To a solution of 5-(2,3,4,5-Tetrahydro-1H-3-benzazepin-7-ylmethyl)-2-pyridinecarbonitrile (may be prepared as described in Description 66) (244 mg, 0.93 mmol) in dichloromethane (5 ml) was added cyclobutanone (0.14 ml, 1.86 mmol) and acetic acid (2 drops) and the resulting mixture was stirred for 20 minutes. The reaction was treated with sodium triacetoxyborohydride (394 mg, 1.86 mmol) and stirred for a further 18 hours. The reaction mixture was diluted with methanol and applied to an SCX ion exchange cartridge and eluted with methanol and 2M ammonia/methanol. The combined basic fractions were evaporated under reduced pressure to afford the title product. MS (AP+) m/e 318 [M+H]⁺.

### EXAMPLES 13-34

### E13-34

The following examples may be prepared from the corresponding amine and cyclobutanone using an analogous method to that described in Example 12:

| Example | Amine | Mass Spectrum |
|---|---|---|
| 3-Cyclobutyl-7-{[4-(3-methyl- | 7-{[4-(3-Methyl-1,2,4- | MS(AP+) m/e |
| 1,2,4-oxadiazol-5- | oxadiazol-5-yl)phenyl]methyl}- | 374[M + H]⁺ |
| yl)phenyl]methyl}-2,3,4,5- | 2,3,4,5-tetrahydro-1H-3- | |
| tetrahydro-1H-3- | benzazepine (may be | |
| benzazepine (E13) | prepared as described in | |
| | Description 20) | |
| 3-Cyclobutyl-7-{[6-(3-methyl- | 7-{[6-(3-Methyl-1,2,4- | MS(AP+) m/e |
| 1,2,4-oxadiazol-5-yl)-3- | oxadiazol-5-yl)-3- | 375[M + H]⁺ |
| pyridinyl]methyl}-2,3,4,5- | pyridinyl]methyl}-2,3,4,5- | |
| tetrahydro-1H-3- | tetrahydro-1H-3-benzazepine | |
| benzazepine (E14) | (may be prepared as | |
| | described in Description 67) | |
| 3-Cyclobutyl-7-{[5-(3-methyl- | 7-{[5-(3-Methyl-1,2,4- | MS(AP+), m/e |
| 1,2,4-oxadiazol-5-yl)-2- | oxadiazol-5-yl)-2- | 376[M + H]⁺ |
| pyrazinyl]methyl}-2,3,4,5- | pyrazinyl]methyl}-2,3,4,5- | |
| tetrahydro-1H-3- | tetrahydro-1H-3-benzazepine | |
| benzazepine (E15) | (may be prepared as | |
| | described in Description 68) | |
| 3-Cyclobutyl-7-{[5-(3-methyl- | 7-{[5-(3-Methyl-1,2,4- | MS(AP+), m/e |
| 1,2,4-oxadiazol-5-yl)-2- | oxadiazol-5-yl)-2- | 375[M + H]⁺ |
| pyridinyl]methyl}-2,3,4,5- | pyridinyl]methyl}-2,3,4,5- | |
| tetrahydro-1H-3- | tetrahydro-1H-3-benzazepine | |
| benzazepine (E16) | (may be prepared as | |
| | described in Description 69) | |
| 4-[(3-Cyclobutyl-2,3,4,5- | N,N-Dimethyl-4-(2,3,4,5- | MS(AP+), m/e |
| tetrahydro-1H-3-benzazepin- | tetrahydro-1H-3-benzazepin-7- | 363[M + H]⁺ |
| 7-yl)methyl]-N,N- | ylmethyl)benzamide (may be | |
| dimethylbenzamide (E17) | prepared as described in | |
| | Description 70) | |
| 3-Cyclobutyl-7-{[4-(1- | 7-{[4-(1- | MS(AP+), m/e |
| piperidinylcarbonyl)phenyl]methyl}- | Piperidinylcarbonyl)phenyl]methyl}- | 403[M + H]⁺ |
| 2,3,4,5-tetrahydro-1H- | 2,3,4,5-tetrahydro-1H-3- | |
| 3-benzazepine (E18) | benzazepine (may be | |
| | prepared as described in | |
| | Description 71) | |
| 3-Cyclobutyl-7-{[4-(4- | 7-{[4-(4- | MS(AP+), m/e |
| morpholinylcarbonyl)phenyl] | Morpholinylcarbonyl)phenyl]methyl}- | 405[M + H]⁺ |
| methyl}-2,3,4,5-tetrahydro- | 2,3,4,5-tetrahydro-1H-3- | |
| 1H-3-benzazepine (E19) | benzazepine (may be | |
| | prepared as described in | |
| | Description 72) | |
| 3-Cyclobutyl-7-{[5-(1- | 7-{[5-(1-Pyrrolidinylcarbonyl)- | MS(AP+), m/e |
| pyrrolidinylcarbonyl)-2- | 2-pyrazinyl]methyl}-2,3,4,5- | 391[M + H]⁺ |
| pyrazinyl] methyl}-2,3,4,5- | tetrahydro-1H-3-benzazepine | |
| tetrahydro-1H-3- | (may be prepared as | |
| benzazepine (E20) | described in Description 73) | |
| 5-[(3-Cyclobutyl-2,3,4,5- | N-(1-Methylethyl)-5-(2,3,4,5- | MS(AP+), m/e |
| tetrahydro-1H-3-benzazepin- | tetrahydro-1H-3-benzazepin-7- | 379[M + H]⁺ |
| 7-yl)methyl]-N-(1- | ylmethyl)-2- | |
| methylethyl)-2- | pyrazinecarboxamide (may be | |
| pyrazinecarboxamide (E21) | prepared as described in | |
| | Description 74) | |
| 5-[(3-Cyclobutyl-2,3,4,5- | N,N-Dimethyl-5-(2,3,4,5- | MS(AP+), m/e |
| tetrahydro-1H-3-benzazepin- | tetrahydro-1H-3-benzazepin-7- | 365[M + H]⁺ |
| 7-yl)methyl]-N,N-dimethyl-2- | ylmethyl)-2- | |
| pyrazinecarboxamide (E22) | pyrazinecarboxamide (may be | |
| | prepared as described in | |
| | Description 75) | |
| 5-[(3-Cyclobutyl-2,3,4,5- | 5-(2,3,4,5-Tetrahydro-1H-3- | MS(AP+), m/e |
| tetrahydro-1H-3-benzazepin- | benzazepin-7-ylmethyl)-N- | 419[M + H]⁺ |
| 7-yl)methyl]-N-(2,2,2- | (2,2,2-trifluoroethyl)-2- | |
| trifluoroethyl)-2- | pyrazinecarboxamide (may be | |
| pyrazinecarboxamide (E23) | prepared as described in | |
| | Description 76) | |
| N-(Cyanomethyl)-5-[(3- | N-(Cyanomethyl)-5-(2,3,4,5- | MS(AP+), m/e |
| cyclobutyl-2,3,4,5- | tetrahydro-1H-3-benzazepin-7- | 376[M + H]⁺ |
| tetrahydro-1H-3-benzazepin- | ylmethyl)-2- | |
| 7-yl)methyl]-2- | pyrazinecarboxamide (may be | |
| pyrazinecarboxamide (E24) | prepared as described in | |
| | Description 77) | |
| 5-[(3-Cyclobutyl-2,3,4,5- | 5-(2,3,4,5-Tetrahydro-1H-3- | MS(AP+), m/e |
| tetrahydro-1H-3-benzazepin- | benzazepin-7-ylmethyl)-2- | 337[M + H]⁺ |
| 7-yl)methyl]-2- | pyrazinecarboxamide (may be | |
| pyrazinecarboxamide (E25) | prepared as described in | |
| | Description 78) | |
| 5-[(3-Cyclobutyl-2,3,4,5- | N-Methyl-5-(2,3,4,5-tetrahydro- | MS(AP+), m/e |
| tetrahydro-1H-3-benzazepin- | 1H-3-benzazepin-7-ylmethyl)- | 350[M + H]⁺ |
| 7-yl)methyl]-N-methyl-2- | 2-pyridinecarboxamide (may | |
| pyridinecarboxamide (E26) | be prepared as described in | |
| | Description 79) | |
| 5-[(3-Cyclobutyl-2,3,4,5- | 5-(2,3,4,5-Tetrahydro-1H-3- | MS(AP+), m/e |
| tetrahydro-1H-3-benzazepin- | benzazepin-7-ylmethyl)-2- | 336[M + H]⁺ |
| 7-yl)methyl]-2- | pyridinecarboxamide (may be | |
| pyridinecarboxamide (E27) | prepared as described in | |
| | Description 80) | |
| 1-{4-[(3-Cyclobutyl-2,3,4,5- | 1-[4-(2,3,4,5-Tetrahydro-1H-3- | MS(AP+), m/e |
| tetrahydro-1H-3-benzazepin- | benzazepin-7- | 375[M + H]⁺ |
| 7-yl)methyl]phenyl}-2- | ylmethyl)phenyl]-2- | |
| pyrrolidinone (E28) | pyrrolidinone (may be | |
| | prepared as described in | |
| | Description 81) | |
| 1-{5-[(3-Cyclobutyl-2,3,4,5- | 1-[5-(2,3,4,5-Tetrahydro-1H-3- | MS(AP+), m/e |
| tetrahydro-1H-3-benzazepin- | benzazepin-7-ylmethyl)-2- | 376[M + H]⁺ |
| 7-yl)methyl]-2-pyridinyl}-2- | pyridinyl]-2-pyrrolidinone (may | |
| pyrrolidinone (E29) | be prepared as described in | |
| | Description 82) | |
| 3-Cyclobutyl-7-(2- | 7-(2-Pyrazinylmethyl)-2,3,4,5- | MS(AP+), m/e |
| pyrazinylmethyl)-2,3,4,5- | tetrahydro-1H-3-benzazepine | 294[M + H]⁺ |
| tetrahydro-1H-3- | (may be prepared as | |
| benzazepine (E30) | described in Description 83) | |
| 1-{5-[(3-Cyclobutyl-2,3,4,5- | 1-[5-(2,3,4,5-Tetrahydro-1H-3- | MS(AP+), m/e |
| tetrahydro-1H-3-benzazepin- | benzazepin-7-ylmethyl)-2- | 377[M + H]⁺ |
| 7-yl)methyl]-2-pyrazinyl}-2- | pyrazinyl]-2-pyrrolidinone (may | |
| pyrrolidinone (E31) | be prepared as described in | |
| | Description 84) | |
| N-{5-[(3-Cyclobutyl-2,3,4,5- | N-[5-(2,3,4,5-Tetrahydro-1H-3- | MS(AP+), m/e |
| tetrahydro-1H-3-benzazepin- | benzazepin-7-ylmethyl)-2- | 351[M + H]⁺ |
| 7-yl)methyl]-2- | pyrazinyl]acetamide (may be | |
| pyrazinyl}acetamide (E32) | prepared as described in | |
| | Description 85) | |

### EXAMPLE 33

### 5-[(3-Cyclobutyl-2,3,4,5-tetrahydro-1H-3-benzazepin-7-yl)methyl]-N-methyl-2-pyrazinecarboxamide (E33)

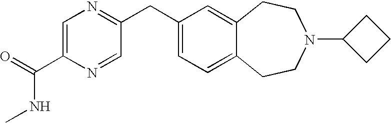 Method A

N-Methyl-5-(2,3,4,5-tetrahydro-1H-3-benzazepin-7-ylmethyl)-2-pyrazinecarboxamide (may be prepared as described in Description 64) (60 mg, 0.20 mmol) was dissolved in dichloromethane (3 ml) and treated with cyclobutanone (0.03 ml, 0.40 mmol), sodium triacetoxyborohydride (85 mg, 0.40 mmol) and acetic acid (1 drop). The reaction mixture was stirred at room temperature under argon for 2 hours. The reaction mixture was diluted with methanol and applied to an SCX ion exchange cartridge and eluted with methanol and 2M ammonia/methanol. The basic fractions were combined and evaporated under reduced pressure to afford the title product. MS (AP+) m/e 351 [M+H]⁺. ¹H NMR (CDCl₃) 9.30 (1H, s), 8.35 (1H, s), 7.70 (1H, s), 7.06-6.99 (3H, m), 4.18 (2H, s), 3.04-3.02 (3H, d), 2.90-2.83 (4H, m), 2.79-2.75 (1H, m), 2.49-2.38 (4H, m), 2.10-2.03 (2H, m), 1.93-1.88 (2H, m), 1.71-1.57 (2H, m).

Method B

N-Methyl-5-(2,3,4,5-tetrahydro-1H-3-benzazepin-7-ylmethyl)-2-pyrazinecarboxamide (may be prepared as described in Description 64) (30 mg, 0.10 mmol) was dissolved in dichloromethane (2 ml), treated with cyclobutanone (0.02 ml, 0.20 mmol) and acetic acid (1 drop) and the resulting mixture was stirred for 20 minutes. The reaction was treated with sodium triacetoxyborohydride (42 mg, 0.20 mmol) and stirred for a further 18 hours. The reaction mixture was diluted with methanol and applied to an SCX column eluting with methanol and 2M ammonia/methanol. The basic fractions were combined and evaporated under reduced pressure to afford the title product. MS (AP+) m/e 351 [M+H]⁺. ¹H NMR (CDCl₃) 9.30 (1H, s), 8.35 (1H, s), 7.70 (1H, s), 7.06-6.99 (3H, m), 4.18 (2H, s), 3.04-3.02 (3H, d), 2.90-2.83 (4H, m), 2.79-2.75 (1H, m), 2.49-2.38 (4H, m), 2.10-2.03 (2H, m), 1.93-1.88 (2H, m), 1.71-1.57 (2H, m).

Method C

N-Methyl-5-(2,3,4,5-tetrahydro-1H-3-benzazepin-7-ylmethyl)-2-pyrazinecarboxamide (may be prepared as described in Description 64) (36 mg, 0.12 mmol) was dissolved in dichloromethane (2 ml), treated with cyclobutanone (0.018 ml, 0.24 mmol) and acetic acid (1 drop) and the resulting mixture was stirred at room temperature. The reaction was treated with sodium triacetoxyborohydride (51 mg, 0.24 mmol) and stirred for 1.5 hours. The solvent was evaporated and the residue was dissolved in methanol and applied to an SCX column eluting with methanol and 2M ammonia/methanol. The product containing fraction was evaporated under reduced pressure to afford the title product. MS (AP+) m/e 351 [M+H]⁺. ¹H NMR (CDCl₃) 9.30 (1H, s), 8.35 (1H, s), 7.70 (1H, s), 7.06-6.99 (3H, m), 4.18 (2H, s), 3.04-3.02 (3H, d), 2.90-2.83 (4H, m), 2.79-2.75 (1H, m), 2.49-2.38 (4H, m), 2.10-2.03 (2H, m), 1.93-1.88 (2H, m), 1.71-1.57 (2H, m).

### EXAMPLE 33A

### 5-[(3-Cyclobutyl-2,3,4,5-tetrahydro-1H-3-benzazepin-7-yl) methyl]-N-methyl-2-pyrazinecarboxamide hydrochloride (E33A)

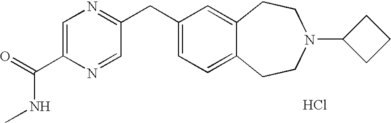

5-[(3-Cyclobutyl-2,3,4,5-tetrahydro-1H-3-benzazepin-7-yl)methyl]-N-methyl-2-pyrazinecarboxamide (may be prepared as described in Example 33, method A) was dissolved in methanol and treated with 1 equivalent of 1 M HCl in diethylether. The mixture was then evaporated under reduced pressure to afford the title product. ¹H NMR (DMSO) 10.65-10.60 (1H, m), 9.06 (1H, s), 8.81-8.80 (1H, d), 8.71 (1H, s), 7.15 (3H, s), 4.19 (2H, s), 3.64-3.41 (1H, m), 3.50-3.40 (2H, m), 3.31-3.22 (2H, m), 2.96-2.91 (2H, m), 2.82-2.81 (3H, d), 2.78-2.67 (2H, m), 2.40-2.30 (2H, m), 2.20-2.14 (2H, m), 1.77-1.60 (2H, m).

### EXAMPLE 34

### 5-[(3-Cyclobutyl-2,3,4,5-tetrahydro-1H-3-benzazepin-7-yl)methyl]-N-ethyl-2-pyrazinecarboxamide (E34)

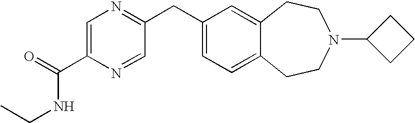

To a solution of N-ethyl-5-(2,3,4,5-tetrahydro-1H-3-benzazepin-7-ylmethyl)-2-pyrazinecarboxamide (may be prepared as described in Description 65) (37 mg, 0.12 mmol) in dichloromethane (3 ml) was added cyclobutanone (0.02 ml, 0.24 mmol), acetic acid (1 drop) and sodium triacetoxyborohydride (51 mg, 0.24 mmol). The resulting mixture was stirred for 1 hour. The reaction mixture was diluted with methanol, applied to an SCX ion exchange cartridge and eluted with methanol and 2M ammonia/methanol. The basic fractions were combined and evaporated to afford the title product. MS (AP+) m/e 365 [M+H]⁺. ¹H NMR (CDCl₃) 9.30 (1H, s), 8.35 (1H, s), 7.69 (1H, s), 7.05-6.99 (3H, m), 4.18 (2H, s), 3.55-3.47 (2H, s), 2.90-2.83 (4H, m), 2.78-2.74 (1H, m), 2.48-2.38 (4H, m), 2.09-2.03 (2H, m), 1.92-1.87 (2H, m), 1.71-1.57 (2H, m), 1.27-1.24 (3H, t).

### EXAMPLES 35-37

### E35-37

The following examples may be prepared from the corresponding amine and cyclopentanone using an analogous method to that described in Example 12:

| Example | Amine | Mass Spectrum |
|---|---|---|
| 5-[(3-Cyclopentyl-2,3,4,5- | N-Methyl-5-(2,3,4,5-tetrahydro- | MS(AP+) m/e |
| tetrahydro-1H-3-benzazepin- | 1H-3-benzazepin-7-ylmethyl)- | 365[M + H]⁺ |
| 7-yl)methyl]-N-methyl-2- | 2-pyrazinecarboxamide (may | |
| pyrazinecarboxamide (E35) | be prepared as described in | |
| | Description 64) | |
| 1-{4-[(3-Cyclopentyl-2,3,4,5- | 1-[4-(2,3,4,5-Tetrahydro-1H-3- | MS(AP+) m/e |
| tetrahydro-1H-3-benzazepin- | benzazepin-7- | 389[M + H]⁺ |
| 7-yl)methyl]phenyl}-2- | ylmethyl)phenyl]-2- | |
| pyrrolidinone (E36) | pyrrolidinone (may be | |
| | prepared as described in | |
| | Description 81) | |
| 1-{5-[(3-cyclopentyl-2,3,4,5- | 1-[5-(2,3,4,5-Tetrahydro-1H-3- | MS(AP+), m/e |
| tetrahydro-1H-3-benzazepin- | benzazepin-7-ylmethyl)-2- | 390[M + H]⁺ |
| 7-yl)methyl]-2-pyridinyl}-2- | pyridinyl]-2-pyrrolidinone (may | |
| pyrrolidinone (E37) | be prepared as described in Description | |
| | 82) | |

### EXAMPLE 38

### 7-[(6-Bromo-3-pyridinyl)methyl]-3-cyclobutyl-2,3,4,5-tetrahydro-1H-3-benzazepine (E38)

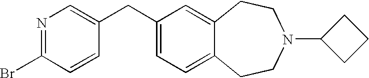

To a solution of 7-[(6-Bromo-3-pyridinyl)methyl]-2,3,4,5-tetrahydro-1H-3-benzazepine (may be prepared as described in Description 87) (177 mg, 0.56 mmol) in dichloromethane (4 ml) was added cyclobutanone (0.08 ml, 1.12 mmol) and acetic acid (2 drops) and the resulting mixture was stirred for 20 minutes at room temperature. The reaction was treated with sodium triacetoxyborohydride (237 mg, 1.12 mmol) and stirred for a further 18 hours at room temperature. The reaction mixture was diluted with methanol and applied to an SCX ion exchange cartridge (Varian bond-elute, 10 g) and washed with methanol and 2M ammonia/methanol. The combined basic fractions were concentrated in vacuo to afford the title product. MS (AP+) m/e 373 [M+2H]⁺.

### EXAMPLE 39

### 3-{5-[(3-Cyclobutyl-2,3,4,5-tetrahydro-1H-3-benzazepin-7-yl)methyl]-2-pyridinyl}-1,3-oxazolidin-2-one (E39)

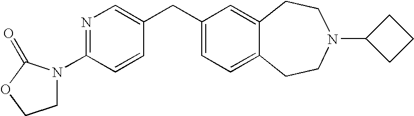

7-[(6-Bromo-3-pyridinyl)methyl]-3-cyclobutyl-2,3,4,5-tetrahydro-1H-3-benzazepine (may be prepared as described in Example 38) (98 mg, 0.26 mmol), oxazolidinone (45 mg, 0.52 mmol), potassium carbonate (130 mg, 0.94 mmol), copper (I) iodide (15 mg, 0.08 mmol) and N,N′-dimethyl-1,2-ethanediamine (0.01 ml, 0.10 mmol) were added together in dry dioxan (3 ml) and the resulting mixture heated at 140° C. in a microwave reactor for 40 minutes. The reaction mixture was diluted with methanol and applied to an SCX ion exchange cartridge (Varian bond-elute, 5 g) and washed with methanol and 2M ammonia/methanol. The combined basic fractions were concentrated in vacuo and the resulting residue was purified by column chromatography eluting with 2M ammonia in methanol:dichloromethane (1:19) to afford the title product. MS (AP+) m/e 378 [M+H]⁺.

### EXAMPLE 40

### 1-{5-[(3-Cyclobutyl-2,3,4,5-tetrahydro-1H-3-benzazepin-7-yl)methyl]-2-pyridinyl}-3-methyl-2-imidazolidinone (E40)

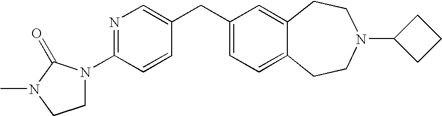

The title compound may be prepared from 7-[(6-bromo-3-pyridinyl)methyl]-3-cyclobutyl-2,3,4,5-tetrahydro-1H-3-benzazepine (may be prepared as described in Example 38) and 1-methyl-2-imidazolidinone using an analogous method to that described in Example 39. MS (AP+) m/e 391 [M+H]⁺.

### EXAMPLE 41

### 6-[(3-cyclobutyl-2,3,4,5-tetrahydro-1H-3-benzazepin-7-yl)methyl]-3-pyridinecarbonitrile (E41)

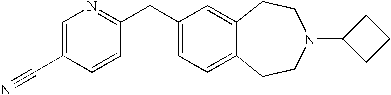

Cyclobutanone (0.02 ml, 0.27 mmol) was added to a solution of 6-(2,3,4,5-tetrahydro-1H-3-benzazepin-7-ylmethyl)-3-pyridinecarbonitrile (may be prepared as described in Description 35) (48.0 mg, 0.18 mmol) in dichloromethane (2 ml) containing glacial acetic acid (1 drop). The mixture was stirred for ˜10 minutes at room temperature, then sodium triacetoxyborohydride (57.0 mg, 0.27 mmol) was added and the mixture stirred at room temperature for 3 hours. The reaction mixture was applied to a SCX cartridge (Varian bond-elute, 2 g) and eluted with methanol followed by a mixture of 2M ammonia/methanol. The basic fractions were combined and evaporated to afford the title compound. MS (ES+) m/e 318 [M+H]⁺.

### EXAMPLE 42

### 6-[(3-cyclobutyl-2,3,4,5-tetrahydro-1H-3-benzazepin-7-yl)methyl]-N-methyl-3-pyridinecarboxamide (E42)

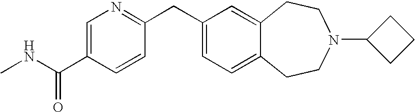

Cyclobutanone (35.0 μl, 0.47 mmol) was added to a solution of N-methyl-6-(2,3,4,5-tetrahydro-1H-3-benzazepin-7-ylmethyl)-3-pyridinecarboxamide (may be prepared as described in Description 88) (90.0 mg, 0.31 mmol) in dichloromethane (4 ml) containing glacial acetic acid (4 drops). The mixture was stirred for 15 minutes at room temperature, then sodium triacetoxyborohydride (99.0 mg, 0.47 mmol) was added and the mixture stirred at room temperature for 2 hours. A further portion of cyclobutanone (35 μl, 0.47 mmol) and sodium triacetoxyborohydride (99.0 mg, 0.47 mmol) was added and the mixture stirred a room temperature for 1 hour. The reaction mixture was applied to a SCX cartridge (Varian bond-elute, 2 g) and washed with methanol followed by a mixture of 2M ammonia/methanol. The basic fractions were combined and evaporated. The product was purified by chromatography on silica, eluting with a gradient of 2M ammonia in methanol/dichloromethane (0-10%) to afford the title compound. MS (ES+) m/e 350 [M+H]⁺.

### EXAMPLES 43-44

### E43-44

Examples 43-44 may be prepared from appropriate amides (D89-D90) and cyclobutanone using an analogous method to that described for Example 42 (see table)

| | | LC/MS |
|---|---|---|
| Example | Amide | (M + H⁺) |
| 3-cyclobutyl-7-{[5-(1- | 7-{[5-(1-pyrrolidinylcarbonyl)-2- | 390 |
| pyrrolidinylcarbonyl)-2- | pyridinyl]methyl}-2,3,4,5- | |
| pyridinyl]methyl}-2,3,4,5-tetrahydro- | tetrahydro-1H-3-benzazepine | |
| 1H-3-benzazepine (E43) | (may be prepared as described | |
| | in Description 89) | |
| 6-[(3-cyclobutyl-2,3,4,5-tetrahydro-1H- | 6-(2,3,4,5-tetrahydro-1H-3- | 336 |
| 3-benzazepin-7-yl)methyl]-3- | benzazepin-7-ylmethyl)-3- | |
| pyridinecarboxamide (E44) | pyridinecarboxamide (may be | |
| | prepared as described in | |
| | Description 90) | |

### EXAMPLE 45

### 4-[(3-cyclobutyl-2,3,4,5-tetrahydro-1H-3-benzazepin-7-yl)methyl]benzoic acid (E45)

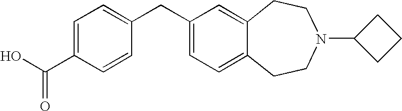

A solution of 4-[(3-cyclobutyl-2,3,4,5-tetrahydro-1H-3-benzazepin-7-yl)methyl]benzonitrile (may be prepared as described in Example 1) (360 mg, 1.14 mmol) in formic acid (3 ml) was treated with concentrated hydrochloric acid (3 ml) and heated at reflux for 24 hours. The reaction mixture was allowed to cool, evaporated to dryness and azeotroped using toluene to yield the title compound. MS (ES+) m/e 336 [M+H]⁺.

### EXAMPLE 46

### 4-[(3-cyclobutyl-2,3,4,5-tetrahydro-1H-3-benzazepin-7-yl)methyl]benzamide (E46)

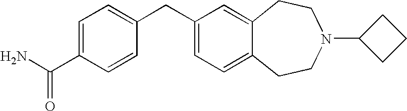

A mixture of 4-[(3-cyclobutyl-2,3,4,5-tetrahydro-1H-3-benzazepin-7-yl)methyl]benzoic acid (may be prepared as described in Example 45) (54.0 mg, 0.16 mmol), polymer bound dicyclohexylcarbodiimide resin (152 mg, 0.32 mmol, 2.1 mmol/g) and 1-hydroxybenzotriazole (43.0 mg, 0.32 mmol) and dimethylformamide (2 ml) were stirred at room temperature for 1 hour. 0.880 ammonia (8.00 mg, 0.48 mmol) was then added and the resulting mixture stirred at room temperature for 18 hours. A further portion of polymer bound dicyclohexylcarbodiimide resin (152 mg, 0.32 mmol, 2.1 mmol/g), 1-hydroxybenzotriazole (43.0 mg, 0.32 mmol) and 0.880 ammonia (8.00 mg, 0.48 mmol) was added and the reaction mixture stirred for 24 hours. The mixture was applied to a SCX cartridge (Varian bond-elute, 5 g) and washed with methanol followed by a mixture of 2M ammonia/methanol. The basic fractions were combined and evaporated. The product was purified by chromatography on silica, eluting with a mixture of 2M ammonia in methanol/dichloromethane (0-10%) to afford the title compound. MS (ES+) m/e 335 [M+H]⁺.

### EXAMPLES 47-51

Examples 47-51 may be prepared from 4-[(3-cyclobutyl-2,3,4,5-tetrahydro-1H-3-benzazepin-7-yl)methyl]benzoic acid (may be prepared as described in Example 45) and the appropriate amine using the analogous method to that described for Example 46 (see table)

| | | LC/MS |
|---|---|---|
| Example | Amine | (M + H⁺) |
| 4-[(3-cyclobutyl-2,3,4,5-tetrahydro-1H-3-benzazepin-7- | methylamine | 349 |
| yl)methyl]-N-methylbenzamide (E47) | | |
| 4-[(3-cyclobutyl-2,3,4,5-tetrahydro-1H-3-benzazepin-7- | ethylamine | 363 |
| yl)methyl]-N-ethylbenzamide (E48) | | |
| 3-cyclobutyl-7-{[4-(1-pyrrolidinylcarbonyl)phenyl]methyl}- | pyrrolidine | 389 |
| 2,3,4,5-tetrahydro-1H-3-benzazepine (E49) | | |
| 4-[(3-cyclobutyl-2,3,4,5-tetrahydro-1H-3-benzazepin-7- | tetrahydro-2H- | 419 |
| yl)methyl]-N-(tetrahydro-2H-pyran-4-yl)benzamide (E50) | pyran-4-amine | |
| 4-[(3-cyclobutyl-2,3,4,5-tetrahydro-1H-3-benzazepin-7- | (1-methylethyl) | 377 |
| yl)methyl]-N-(1-methylethyl)benzamide (E51) | amine | |

### EXAMPLE 52

### 5-[(3-cyclobutyl-2,3,4,5-tetrahydro-1H-3-benzazepin-7-yl)methyl]-2-pyridinecarboxylic acid (E52)

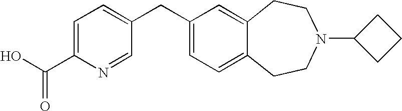

A solution of 5-[(3-cyclobutyl-2,3,4,5-tetrahydro-1H-3-benzazepin-7-yl)methyl]-2-pyridinecarbonitrile (may be prepared as described in Example 12) (230 mg, 0.72 mmol) in formic acid (2 ml) and concentrated hydrochloric acid (2 ml) was heated at reflux for 24 hours. The solvent was evaporated and the product was azeotroped using toluene to remove water. The reaction mixture was then dried in a vacuum oven at 40° C. to yield the title compound. MS (ES+) m/e 337 [M+H]⁺, 293 [M-CO₂]⁺

### EXAMPLE 53

### 5-[(3-cyclobutyl-2,3,4,5-tetrahydro-1H-3-benzazepin-7-yl)methyl]-N-(1-methylethyl)-2-pyridinecarboxamide (E53)

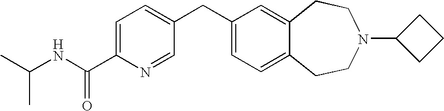

A mixture of 5-[(3-cyclobutyl-2,3,4,5-tetrahydro-1H-3-benzazepin-7-yl)methyl]-2-pyridinecarboxylic acid (may be prepared as described in Example 52) (50.0 mg, 0.15 mmol, 1 equivalent), polymer bound dicyclohexylcarbodiimide resin (152 mg, 0.30 mmol, 2 equivalents, 2.1 mmol/g) and 1-hydroxybenzotriazole (41.0 mg, 0.30 mmol, 2 equivalents) and dimethylformamide (2 ml) were stirred at room temperature for 1 hour. A suspension of (1-methylethyl)amine (27.0 mg, 0.45 mmol, 3 equivalents) in dimethylformamide (0.5 ml) was then added and the resulting mixture stirred at room temperature overnight. A further equivalent of polymer bound dicyclohexylcarbodiimide resin, 1-hydroxybenzotriazole and (1-methylethyl)amine was added and the reaction mixture stirred for 24 hours. The mixture was applied to a 5 g SCX cartridge and eluted with methanol followed by a mixture of 2M ammonia/methanol. The basic, product containing fractions were combined and evaporated. The product was dissolved in a minimum volume of dichloromethane and loaded onto a 25+5 Biotage silica cartridge, eluting with a 0-5% gradient of 2M ammonia in methanol/dichloromethane. The product containing fractions were combined and evaporated to afford the title compound. MS (ES+) m/e 378 [M+H]⁺.

### EXAMPLES 54-56

### E54-56

Examples 54-56 were prepared from 5-[(3-cyclobutyl-2,3,4,5-tetrahydro-1H-3-benzazepin-7-yl)methyl]-2-pyridinecarboxylic acid (may be prepared as described in Example 52) and the appropriate amine using the analogous method to that described for Example 53 (see table)

| | | LC/MS |
|---|---|---|
| Example | Amine | (M + H⁺) |
| 3-cyclobutyl-7-{[6-(1-pyrrolidinylcarbonyl)-3- | pyrrolidine | 390 |
| pyridinyl]methyl}-2,3,4,5-tetrahydro-1H-3-benzazepine | | |
| (E54) | | |
| 5-[(3-cyclobutyl-2,3,4,5-tetrahydro-1H-3-benzazepin-7- | tetrahydro-2H- | 420 |
| yl)methyl]-N-(tetrahydro-2H-pyran-4-yl)-2- | pyran-4-amine | |
| pyridinecarboxamide (E55) | | |
| N-(4-cyanophenyl)-5-[(3-cyclobutyl-2,3,4,5-tetrahydro- | 4- | 437 |
| 1H-3-benzazepin-7-yl)methyl]-2-pyridinecarboxamide | aminobenzonitrile | |
| (E56) | | |

### EXAMPLE 57

### 3-cyclobutyl-7-{[4-(1,2,3-thiadiazol-4-yl)phenyl]methyl}-2,3,4,5-tetrahydro-1H-3-benzazepine (E57)

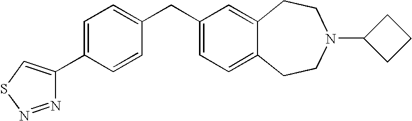

Cyclobutanone (49.0%, 0.65 mmol) was added to a solution of 7-{[4-(1,2,3-thiadiazol-4-yl)phenyl]methyl}-2,3,4,5-tetrahydro-1H-3-benzazepine ((may be prepared as described in Description 95) (140 mg, 0.43 mmol) in dichloromethane (2 ml) containing glacial acetic acid (1%). The mixture was stirred for 1 hour at room temperature, then sodium triacetoxyborohydride (138 mg, 0.65 mmol) was added and the mixture stirred at room temperature for 2 hours. The reaction was quenched with methanol and the mixture applied to a SCX cartridge (Varian bond-elute, 5 g) and washed with methanol followed by a mixture of 2M ammonia/methanol. The basic fractions were combined and evaporated. The product was purified by chromatography on silica, eluting with a mixture of 2M ammonia in methanol/dichloromethane (0-5%) to afford the title compound. MS (ES+) m/e 376 [M+H]⁺.

### EXAMPLES 58-62

### E58-62

Examples 58-62 were prepared from the appropriate benzazepine (D96-D98, D103 and D106) and cyclobutanone using an analogous method to that described for Example 57 (see table)

| | | LC/MS |
|---|---|---|
| Example | benzazepine | (M + H⁺) |
| 3-{4-[(3-cyclobutyl-2,3,4,5-tetrahydro-1H-3- | 3-[4-(2,3,4,5-tetrahydro-1H-3- | 376 |
| benzazepin-7-yl)methyl]Phenyl}-1,2,4- | benzazepin-7-ylmethyl)phenyl]- | |
| oxadiazol-5(2H)-one (E58) | 1,2,4-oxadiazol-5(2H)-one (may | |
| | be prepared as described in | |
| | Description 96) | |
| 3-cyclobutyl-7-{[4-(1H-pyrazol-1- | 7-{[4-(1H-pyrazol-1- | 358 |
| yl)phenyl]methyl}-2,3,4,5-tetrahydro-1H-3- | yl)phenyl]methyl}-2,3,4,5- | |
| benzazepine (E59) | tetrahydro-1H-3-benzazepine | |
| | (may be prepared as described | |
| | in Description 97) | |
| 3-cyclobutyl-7-{[4-(1H-1,2,4-triazol-1- | 7-{[4-(1H-1,2,4-triazol-1- | 359 |
| yl)phenyl]methyl}-2,3,4,5-tetrahydro-1H-3- | yl)phenyl]methyl}-2,3,4,5- | |
| benzazepine (E60) | tetrahydro-1H-3-benzazepine | |
| | (may be prepared as described | |
| | in Description 98) | |
| 1-[(3-cyclobutyl-2,3,4,5-tetrahydro-1H-3- | N-methyl-1-(2,3,4,5-tetrahydro- | 339 |
| benzazepin-7-yl)methyl]-N-methyl-1H- | 1H-3-benzazepin-7-ylmethyl)- | |
| imidazole-5-carboxamide (E61) | 1H-imidazole-5-carboxamide | |
| | (may be prepared as described | |
| | in Description 103) | |
| 1-[(3-cyclobutyl-2,3,4,5-tetrahydro-1H-3- | N-methyl-1-(2,3,4,5-tetrahydro- | 339 |
| benzazepin-7-yl)methyl]-N-methyl-1H- | 1H-3-benzazepin-7-ylmethyl)- | |
| imidazole-4-carboxamide (E62) | 1H-imidazole-4-carboxamide | |
| | (may be prepared as described | |
| | in Description 106) | |

Biological Data

A membrane preparation containing histamine H3 receptors may be prepared in accordance with the following procedures:

(i) Generation of Histamine H3 Cell Line

DNA encoding the human histamine H3 gene (Huvar, A. et al. (1999) Mol. Pharmacol. 55(6), 1101-1107) was cloned into a holding vector, pcDNA3.1 TOPO (InVitrogen) and its cDNA was isolated from this vector by restriction digestion of plasmid DNA with the enzymes BamH1 and Not-1 and ligated into the inducible expression vector pGene (InVitrogen) digested with the same enzymes. The GeneSwitch™ system (a system where in transgene expression is switched off in the absence of an inducer and switched on in the presence of an inducer) was performed as described in U.S. Pat. Nos. 5,364,791; 5,874,534; and 5,935,934. Ligated DNA was transformed into competent DH5α E. coli host bacterial cells and plated onto Luria Broth (LB) agar containing Zeocin™ (an antibiotic which allows the selection of cells expressing the sh ble gene which is present on pGene and pSwitch) at 50 μg ml⁻¹. Colonies containing the re-ligated plasmid were identified by restriction analysis. DNA for transfection into mammalian cells was prepared from 250 ml cultures of the host bacterium containing the pGeneH3 plasmid and isolated using a DNA preparation kit (Qiagen Midi-Prep) as per manufacturers guidelines (Qiagen). CHO K1 cells previously transfected with the pSwitch regulatory plasmid (InVitrogen) were seeded at 2×10e6 cells per T75 flask in Complete Medium, containing Hams F12 (GIBCOBRL, Life Technologies) medium supplemented with 10% v/v dialysed foetal bovine serum, L-glutamine, and hygromycin (100 μg ml⁻¹), 24 hours prior to use. Plasmid DNA was transfected into the cells using Lipofectamine plus according to the manufacturers guidelines (InVitrogen). 48 hours post transfection cells were placed into complete medium supplemented with 500 μg ml⁻¹ Zeocin™.

10-14 days post selection 10 nM Mifepristone (InVitrogen), was added to the culture medium to induce the expression of the receptor. 18 hours post induction cells were detached from the flask using ethylenediamine tetra-acetic acid (EDTA; 1:5000; InVitrogen), following several washes with phosphate buffered saline pH 7.4 and resuspended in Sorting Medium containing Minimum Essential Medium (MEM), without phenol red, and supplemented with Earles salts and 3% Foetal Clone II (Hyclone). Approximately 1×10e7 cells were examined for receptor expression by staining with a rabbit polyclonal antibody, 4a, raised against the N-terminal domain of the histamine H3 receptor, incubated on ice for 60 minutes, followed by two washes in sorting medium. Receptor bound antibody was detected by incubation of the cells for 60 minutes on ice with a goat anti rabbit antibody, conjugated with Alexa 488 fluorescence marker (Molecular Probes). Following two further washes with Sorting Medium, cells were filtered through a 50 μm Filcon™ (BD Biosciences) and then analysed on a FACS Vantage SE Flow Cytometer fitted with an Automatic Cell Deposition Unit. Control cells were non-induced cells treated in a similar manner. Positively stained cells were sorted as single cells into 96-well plates, containing Complete Medium containing 500 μg ml⁻¹ Zeocin™ and allowed to expand before reanalysis for receptor expression via antibody and ligand binding studies. One clone, 3H3, was selected for membrane preparation.

(ii) Membrane Preparation from Cultured Cells

All steps of the protocol are carried out at 4° C. and with pre-cooled reagents. The cell pellet is resuspended in 10 volumes of homogenisation buffer (50 mM N-2-hydroxyethylpiperazine-N′-2-ethanesulfonic acid (HEPES), 1 mM ethylenediamine tetra-acetic acid (EDTA), pH 7.4 with KOH, supplemented with 10e-6M leupeptin (acetyl-leucyl-leucyl-arginal; Sigma L2884), 25 μg/ml bacitracin (Sigma B0125), 1 mM phenylmethylsulfonyl fluoride (PMSF) and 2×10e-6M pepstain A (Sigma)). The cells are then homogenised by 2×15 second bursts in a 1 litre glass Waring blender, followed by centrifugation at 500 g for 20 minutes. The supernatant is then spun at 48,000 g for 30 minutes. The pellet is resuspended in homogenisation buffer (4× the volume of the original cell pellet) by vortexing for 5 seconds, followed by homogenisation in a Dounce homogeniser (10-15 strokes). At this point the preparation is aliquoted into polypropylene tubes and stored at −80° C.

(iii) Generation of Histamine H1 Cell Line

The human H1 receptor was cloned using known procedures described in the literature [Biochem. Biophys. Res. Commun. 1994, 201(2), 894]. Chinese hamster ovary cells stably expressing the human H1 receptor were generated according to known procedures described in the literature [Br. J. Pharmacol. 1996, 117(6), 1071].

Compounds of the invention may be tested for in vitro biological activity in accordance with the following assays:

(II) Histamine H3 Functional Antagonist Assay

For each compound being assayed, in a solid white 384 well plate, is added:—

(a) 0.5 μl of test compound diluted to the required concentration in DMSO (or 0.5 μl DMSO as a control);

(b) 30 μl bead/membrane/GDP mix prepared by mixing Wheat Germ Agglutinin Polystyrene LeadSeeker® (WGA PS LS) scintillation proximity assay (SPA) beads with membrane (prepared in accordance with the methodology described above) and diluting in assay buffer (20 mM N-2-Hydroxyethylpiperazine-N′-2-ethanesulfonic acid (HEPES)+100 mM NaCl+10 mM MgCl₂, pH7.4 NaOH) to give a final volume of 30 μl which contains 5 μg protein and 0.25 mg bead per well, incubating at room temperature for 60 minutes on a roller and, just prior to addition to the plate, adding 10 M final concentration of guanosine 5′ diphosphate (GDP) (Sigma; diluted in assay buffer);

(c) 15 μl 0.38 nM [³⁵S]-GTPγS (Amersham; Radioactivity concentration=37 MBq/ml; Specific activity=1160 Ci/mmol), histamine (at a concentration that results in the final assay concentration of histamine being EC₈₀).

After 2-6 hours, the plate is centrifuged for 5 min at 1500 rpm and counted on a Viewlux counter using a 613/55 filter for 5 min/plate. Data is analysed using a 4-parameter logistical equation. Basal activity used as minimum i.e. histamine not added to well.

(III) Histamine H1 Functional Antagonist Assay

The histamine H1 cell line was seeded into non-coated black-walled clear bottom 384-well tissue culture plates in alpha minimum essential medium (Gibco/Invitrogen, cat no. 22561-021), supplemented with 10% dialysed foetal calf serum (Gibco/Invitrogen cat no. 12480-021) and 2 mM L-glutamine (Gibco/Invitrogen cat no 25030-024) and maintained overnight at 5% CO₂, 37° C.

Excess medium was removed from each well to leave 10 μl. 30 μl loading dye (250 μM Brilliant Black, 2 μM Fluo-4 diluted in Tyrodes buffer+probenecid (145 mM NaCl, 2.5 mM KCl, 10 mM HEPES, 10 mM D-glucose, 1.2 mM MgCl₂, 1.5 mM CaCl₂, 2.5 mM probenecid, pH adjusted to 7.40 with NaOH 1.0 M)) was added to each well and the plates were incubated for 60 minutes at 5% CO₂, 37° C.

10 μl of test compound, diluted to the required concentration in Tyrodes buffer+probenecid (or 10 μl Tyrodes buffer+probenecid as a control) was added to each well and the plate incubated for 30 min at 37° C., 5% CO₂. The plates were then placed into a FLIPR™ (Molecular Devices, UK) to monitor cell fluorescence (λₑₓ=488 nm, λ_{EM}=540 nm) in the manner described in Sullivan et al. (In: Lambert DG (ed.), Calcium Signaling Protocols, New Jersey Humana Press, 1999, 125-136) before and after the addition of 10 μl histamine at a concentration that results in the final assay concentration of histamine being EC₈₀.

Functional antagonism is indicated by a suppression of histamine induced increase in fluorescence, as measured by the FLIPR™ system (Molecular Devices). By means of concentration effect curves, functional affinities are determined using standard pharmacological mathematical analysis.

Results

The compounds of Examples E1-E3, E5-E24, E26-E37, E39-E44, E46, E48-E51 and E53-E62 were tested in the histamine H3 functional antagonist assay. The results are expressed as functional pKᵢ (fpKᵢ) values. A functional pKi is the negative logarithm of the antagonist equilibrium dissociation constant as determined in the H3 functional antagonist assay using membrane prepared from cultured H3 cells. The results given are averages of a number of experiments. All compounds tested exhibited antagonism >6.5 fpKᵢ, more particularly the compounds of Examples E1, E5, E12-E24, E26-E37, E39-E44, E46, E48-E51, E53-E60 exhibited antagonism >8.5 fpkᵢ. Even more particularly, the compounds of Examples E1, E13, E31, E33, E46, E57 and E59-E60 exhibited antagonism >9.5 fpkᵢ.

The compounds of Examples E1-E3, E5-E37, E39-E44, E46-E49, E51, E53-E55 and E61-E62 were tested in the histamine H1 functional antagonist assay. Again, the results are expressed as functional pKᵢ (fpKᵢ) values and are averages of a number of experiments. The functional pKi may be derived from the negative logarithm of the pIC50 (concentration producing 50% inhibition) in the H1 functional antagonist assay according to the Cheng-Prusoff equation (Cheng, Y. C. and Prusoff, W. H., 1973, Biochem. Pharmacol. 22, 3099-3108). All compounds tested exhibited antagonism <6.0 fpkᵢ.

What is claimed is:

1-24. (canceled)

25. A compound of formula (I) or a pharmaceutically acceptable salt thereof: 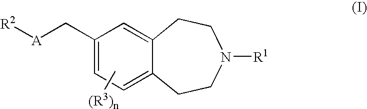
wherein
R¹ represents C₁₋₆ alkyl of —C₃₋₇ cycloalkyl, wherein the C₃₋₇ cycloalkyl group may optionally be substituted by C₁₋₃ alkyl;
A represents a bond, O, S or NR⁷;
R⁷ represents hydrogen, C₁₋₆ alkyl or aryl;
R² represents -aryl, -heteroaryl, —C₃₋₈ cycloalkyl-Y—C₃₋₈ cycloalkyl, —C₃₋₆ cycloalkyl-Y-aryl, —C₃₋₈ cycloalkyl-Y-heteroaryl, —C₃₋₈ cycloalkyl-Y—C₃₋₈ heterocyclyl, -aryl-Y—C₃₋₈ cycloalkyl, -aryl-Y-aryl, -aryl-Y-heteroaryl, -aryl-Y-heterocyclyl, -heteroaryl-Y—C₃₋₈ cycloalkyl, heteroaryl-Y-aryl, -heteroaryl-Y-heteroaryl, -heteroaryl-Y-heterocyclyl -heteroaryl-Y—C₃₋₈ cycloalkyl, -heterocyclyl-Y-aryl, -heterocyclyl-Y-heteroaryl or -heterocyclyl-Y-hetercyclyl, such that R² is linked to A via a carbon atom;
Y represents a bond, C₁₋₆ alkyl, CO, CONH, COC₂₋₆ alkenyl, O, SO₂ or NHCOC₁₋₆ alkyl;
R³ represents halogen, C₁₋₆ alkyl, C₁₋₆ alkoxy, cyano, amino or trifluoromethyl;
n is 0, 1 or 2;
wherein said alkyl, cycloalkyl, aryl, heteroaryl and heterocyclyl groups of R² may be optionally substituted by one or more substituents (e.g. 1, 2 or 3) which may be the same or different, and which are selected from the group consisting of halogen, hydroxy, cyano, nitro, ═O, SO₂H, —R⁴, —CO₂R⁴, —COR⁴, —NR⁵R⁶, —C₁₋₆ alkyl-NR⁵R⁶, —C₃₋₈ cycloalkyl-NR⁵R⁶, —CONR⁵R⁶, —NR⁵COR⁶, —NR⁵SO₂R⁶, —OCONR⁵R⁶, —NR⁵CO₂R⁶, —CONR⁵R⁶, —NR⁵COR⁶, -alkyl-OR⁸, —SOR⁸, —OR⁹, —SO₂R⁹, —OSO₂R⁹, -alkyl-SO₂R⁹, -alkyl-CONHR⁹, -alkyl-SONHR⁸, -alkyl-COR¹⁰, —CO-alkyl-R¹⁰, —O-alkyl-R¹¹ (wherein R⁴, R⁵ and R⁶ independently represent hydrogen, C₁₋₆ alkyl, —C₃₋₈ cycloalkyl, —C₁₋₆ alkyl-C₃₋₈ cycloalkyl, aryl, heterocyclyl or heteroaryl, wherein R⁸ represents C₁₋₆ alkyl. wherein R⁹ represents C₁₋₆ alkyl or aryl, wherein R¹⁰ represents a nitrogen containing heterocyclyl group);
wherein said R⁴, R⁵, R⁶, R⁸, R⁹, R¹⁰ and R¹¹ groups may be optionally substituted by one or more substituents (e.g. 1, 2 or 3) which may be the same or different, and which are selected from the group consisting of halogen, hydroxy, C₁₋₆ alkyl, C₁₋₆ alkoxy, cyano, amino, ═O or trifluoromethyl;
or solvates thereof.

26. A compound according to claim 25, wherein R¹ represents unsubstituted cyclobutyl or cyclopentyl.

27. A compound according to claim 25, wherein A represents a bond.

28. A compound according to claim 25, wherein R² represents:
-aryl;
-heteroaryl;
-aryl-Y-heteroaryl;
-heteroaryl-Y-heteroaryl;
-aryl-Y-heterocyclyl; or
-heteroaryl-Y-heterocyclyl.

29. A compound according to claim 25, wherein the aryl, heteroaryl and heterocyclyl groups of R² may optionally be substituted by one or more substituents which may be the same or different, and which are selected from the group consisting of halogen, hydroxy, cyano, nitro, ═O, —R⁴ and —CONR⁵R⁶.

30. A compound according to claim 25, wherein Y represents a bond, CO or CONH.

31. A compound according to claim 25, wherein R² represents:
-aryl optionally substituted by one or more substituents selected form cyano, —R⁴ and —CONR⁵R⁶;
-heteroaryl optionally substituted by one or more substituents selected form halogen, cyano, —CO₂R⁴ and —CONR⁵R⁶;
-aryl-Y-heteroaryl optionally substituted by one or more substituents selected from —R⁴ or ═O groups;
-heteroaryl-Y-heteroaryl optionally substituted by one or more —R⁴ groups;
-aryl-Y-heterocyclyl optionally substituted by one or more ═O groups; or
-heteroaryl-Y-heterocyclyl optionally substituted by one or more substituents selected from —R⁴ or ═O groups.

32. A compound according to claim 25, wherein R⁴, R⁵ and R⁶ are independently selected from H or C₁₋₆alkyl.

33. A compound according to claim 25, wherein n represents 0.

34. A compound according to claim 25 which is:
4-[(3-Cyclobutyl-2,3,4,5-tetrahydro-1H-3-benxaxepin-7-yl)methyl]benzonitrile;
5-{[(3-Cyclobutyl-2,3,4,5-tetrahydro-1H-3-benxaxepin-7-yl)methyl]oxy}-N-methyl-2-pyrazinecarboxamide;
5-{[(3-Cyclopentyl-2,3,4,5-tetrahydro-1H-3-benzazepin-7-yl)methyl]oxy}-N-methyl-2-pyrazinecarboxamide;
7-[(5-Bromo-2-pyridinyl)methyl]-3-cyclobutyl-2,3,4,5-tetrahydro-1H-3-benzazepine;
1-{6-[(3-Cyclobutyl-2,3,4,5-tetrahydro-1H-3-benzazepin-7-yl)methyl]-3-pyridinyl}-2-pyrrolidinone;
6-{[(3-Cyclobutyl-2,3,4,5-tetrahydro-1H-3-benzazepin-7-yl)methyl]oxy}-N-methyl-3-pyridinecarboxamide;
6-{[(3-Cyclobutyl-2,3,4,5-tetrahydro-1H-3-benzazepin-7-yl)methyl]oxy}-N-methyl-3-pyridinecarboxamide;
N-Methyl-6-({[3-(2-methylpropyl)-2,3,4,5-tetrahydro-1H-3-benzazepin-7-yl]methyl}oxy)-3-pyridinecarboxamide;
6-{[(3-Cyclobutyl-2,3,4,5-tetrahydro-1H-3-benzazepin-7-yl)methyl]oxy}-N-methyl-3-pyridazinecarboxamide;
6-{[(3-Cyclopentyl-2,3,4,5-tetrahydro-1H-3-benzazepin-7-yl)methyl]oxy}-N-methyl-3-pyridazinecarboxamide;
N-Methyl-6-({[3-(2-methylpropyl)-2,3,4,5-tetrahydro-1H-3-benzazepin-7-yl]methyl}oxy)-3-pyridazine carboxamide;
5-[(3-Cyclobutyl-2,3,4,5-tetrahydro-1H-3-benzazepin-7-yl)methyl-2-pyridinecarbonitrile;
3-Cyclobutyl-7-{[4-(3-methyl-1,2,4-oxadiazol-5-yl)phenyl]methyl}-2,3,4,5-tetrahydro-1H-3-benzazepine;
3-Cyclobutyl-7-{[6-(3-methyl-1,2,4-oxadiazol-5-yl)-3-pyridinyl]methyl}-2,3,4,5-tetrahydro-1H-3-benzazepine;
3-Cyclobutyl-7-{[5-(3-methyl-1,2,4-oxadiazol-5-yl)-2-pyrazinyl]methyl}-2,3,4,5-tetrahydro-1H-3-benzazepine;
4-[(3-Cyclobutyl-2,3,4,5-tetrahydro-1H-3-benzazepin-7-yl)methyl]-N,N-dimethylbenzamide;
3-Cyclobutyl-7-{[4-(1-piperidinylcarbonyl)phenyl]methyl}-2,3,4,5-tetrahydro-1H-3-benzazepine;
3-Cyclobutyl-7-{[4-(4-morpholinylcarbonyl)phenyl]methyl}-2,3,4,5-tetrahydro-1H-3-benzazepine;
5-[(3-Cyclobutyl-2,3,4,5-tetrahydro-1H-3-benzazepin-7-yl)methyl]-N-(1-methylethyl)-2-pyrazinecarboxamide;
5-[(3-Cyclobutyl-2,3,4,5-tetrahydro-1H-3-benzazepin-7-yl)methyl]-N,N-dimethyl-2-pyrazinecarboxamide;
5-[(3-Cyclobutyl-2,3,4,5-tetrahydro-1H-3-benzazepin-7-yl)methyl]-N-(2,2,2-trifluoroethyl)-2-pyrazinecarboxamide;
N-(Cyanomethyl)-5-[(3-cyclobutyl-2,3,4,5-tetrahydro-1H-3-benzazepin-7-yl)methyl]-2-pyrazinecarboxamide;
5-[(3-Cyclobutyl-2,3,4,5-tetrahydro-1H-3-benzazepin-7-yl)methyl]-2-pyrazinecarboxamide;
5-[(3-Cyclobutyl-2,3,4,5-tetrahydro-1H-3-benzazepin-7-yl)methyl]-N-methyl-2-pyrazinecarboxamide;
5-[(3-Cyclobutyl-2,3,4,5-tetrahydro-1H-3-benzazepin-7-yl)methyl]-2-pyridinecarboxamide;
1-{4-[(3-Cyclobutyl-2,3,4,5-tetrahydro-1H-3-benzazepin-7-yl)methyl]phenyl}-2-pyrrolidinone;
1-{5-[(3-Cyclobutyl-2,3,4,5-tetrahydro-1H-3-benzazepin-7-yl)methyl]-2-pyridinyl}-2-pyrrolidinone;
3-Cyclobutyl-7(2-pyrazinylmethyl)-2,3,4,5-tetrahydro-1H-3-benzazepine;
1-{5-[(3-Cyclobutyl-2,3,4,5-tetrahydro-1H-3-benzazepin-7-yl)methyl]-2-pyrazinyl}-2-pyrrolidinone;
N-{5-[(3-Cyclobutyl-2,3,4,5-tetrahydro-1H-3-benzazepin-7-yl)methyl]-2-pyrazinyl}acetamide;
5-[(3-Cyclobutyl-2,3,4,5-tetrahydro-1H-3-benzazepin-7-yl)methyl]-N-methyl-2-pyrazinecarboxamide;
5-[(3-Cyclobutyl-2,3,4,5-tetrahydro-1H-3-benzazepin-7-yl)methyl]-N-ethyl-2-pyrazinecarboxamide;
5-[(3-Cyclobutyl-2,3,4,5-tetrahydro-1H-3-benzazepin-7-yl)methyl]-N-methyl-2-pyrazinecarboxamide;
1-{4-[(3-Cyclopentyl-2,3,4,5-tetrahydro-1H-3-benzazepin-7-yl)methyl]phenyl}-2-pyrrolidinone;
1-{5-[(3-Cyclopentyl-2,3,4,5-tetrahydro-1H-3-benzazepin-7-yl)methyl]-2-pyridinyl}-2-pyrrolidinone;
7-[(6-Bromo-2-pyridinyl)methyl]-3-cyclobutyl-2,3,4,5-tetrahydro-1H-3-benzazepine;
3-{5-[(3-Cyclobutyl-2,3,4,5-tetrahydro-1H-3-benzazepin-7-yl)methyl]-2-pyridinyl}-1,3-oxazolidin-2-one;
1-{5-[(3-Cyclobutyl-2,3,4,5-tetrahydro-1H-3-benzazepin-7-yl)methyl]-2-pyridinyl}-3-methyl-2-imidazolidinone;
6-[(3-Cyclobutyl-2,3,4,5-tetrahydro-1H-3-benzazepin-7-yl)methyl]-3-pyridinecarbonitrile;
3-Cyclobutyl-7-{[5-(1-pyrrolidinylcarbonyl)-2-pyridinyl]methyl}-2,3,4,5-tetrahydro-1H-3-benzazepine;
6-[(3-Cyclobutyl-2,3,4,5-tetrahydro-1H-3-benzazepin-7-yl)methyl]-3-pyridinecarboxamide;
4-[(3-Cyclobutyl-2,3,4,5-tetrahydro-1H-3-benzazepin-7-yl)methyl]benzoic acid;
4-[(3-Cyclobutyl-2,3,4,5-tetrahydro-1H-3-benzazepin-7-yl)methyl]benzamide;
4-[(3-Cyclobutyl-2,3,4,5-tetrahydro-1H-3-benzazepin-7-yl)methyl]-N-methylbenzamide;
4-[(3-Cyclobutyl-2,3,4,5-tetrahydro-1H-3-benzazepin-7-yl)methyl]-N-ethylbenzamide;
3-Cyclobutyl-2,3,4,5-tetrahydro-1H-3-benzazepin-7-yl)methyl]-N-methylbenzamide;
3-Cyclobutyl-7-{[4-(1-pyrrolidinylcarbonyl)phenyl]methyl}-2,3,4,5-tetrahydro-1H-3-benzazepine;
4-[(3-Cyclobutyl-2,3,4,5-tetrahydro-1H-3-benzazepin-7-yl)methyl]-N-(tetrahydro-2H-pyran-4-yl)benzamide;
4-[(3-Cyclobutyl-2,3,4,5-tetrahydro-1H-3-benzazepin-7-yl)methyl]-N-(1-methylethyl)benzamide;
5-[(3-Cyclobutyl-2,3,4,5-tetrahydro-1H-3-benzazepin-7-yl)methyl]-2-pyridinecarboxylic acid;
5-[(3-Cyclobutyl-2,3,4,5-tetrahydro-1H-3-benzazepin-7-yl)methyl]-N-(1-methylethyl)-2-pyridinecarboxamide;
3-Cyclobutyl-7-{[6-(1-pyrrolidinylcarbonyl)-3-pyridinyl]methyl}-2,3,4,5-tetrahydro-1H-3-benzazepine;
5-[(3-Cyclobutyl-2,3,4,5-tetrahydro-1H-3-benzazepin-7-yl)methyl]-N-(tetrahydro-2H-pyran-4-yl)-2-pyridinecarboxamide;
N-(4-Cyanophethyl)-5-[(3-cyclobutyl-2,3,4,5-tetrahydro-1H-3-benzazepin-7-yl)methyl]-2-pyridinecarboxamide;
3-Cyclobutyl-7-{[4-(1,2,3-thiadiazol-4-yl)phenyl]methyl}-2,3,4,5-tetrahydro-1H-3-benzazepine;
3-{4-[(3-Cyclobutyl-2,3,4,5-tetrahydro-1H-3-benzazepin-7-yl)methyl]phenyl}-1,2,4-oxadiazol-5(2H)-one;
3-Cyclobutyl-7-{[4-(1H-pyrazol-1-yl)phenyl]methyl}-2,3,4,5-tetrahydro-1H-3-benzazepine;
3-Cyclobutyl-7-{[4-(1H-1,2,4-triazol-1-yl)phenyl]methyl}-2,3,4,5-tetrahydro-1H-3-benzazepine;
1-[(3-Cyclobutyl-2,3,4,5-tetrahydro-1H-3-benzazepin-7-yl)methyl]-N-methyl-1H-imidazole-5-carboxamide;
1-[(3-Cyclobutyl-2,3,4,5-tetrahydro-1H-3-benzazepin-7-yl)methyl]-N-methyl-1H-imidazole-4-carboxamide;
or a pharmaceutically acceptable salt thereof.

35. A pharmaceutical composition which comprises the compound of claim 25 or a pharmaceutically acceptable salt thereof and a pharmaceutically acceptable carrier or excipient.

36. A method of treatment of neurological diseases which comprises administering to a host in need thereof an effective amount of a compound of claim 25 or a pharmaceutically acceptable salt thereof.

37. A process for the preparation of the compound of formula (I) as defined in claim 25 or a pharmaceutically acceptable salt thereof, which process comprises:
(a) reacting a compound of formula (II) 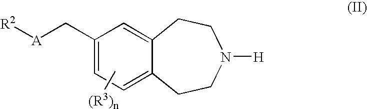
wherein R², R³, A and n are as defined in claim 25, with a compound of formula R^{1′}-L¹, wherein R^{1′} is as defined in claim 25 for R¹ or a group convertible thereto and L¹ represents a suitable leaving group such as a halogen atom;
(b) reacting a compound of formula (II) as defined in claim 25, with a ketone of formula R^{1″}=O, wherein R^{1″} is C₁₋₆ alkyl or C₃₋₇ cycloalkyl, wherein the C₃₋₇ cycloalkyl group may optionally be substituted by C₁₋₃ alkyl;
(c) hydrolysis decarboxylation of a compound of formula (XI) in which A is a bond and R² is -heteroaryl, -heteroaryl-Y-aryl, heteroaryl-Y-heteroaryl, -heteroaryl-Y-heterocyclyl or heteroaryl-Y—C₃₋₈cycloalkyl, wherein the heteroaryl ring attached to A contains a nitrogen atom ortho to the carbon bonded to A. 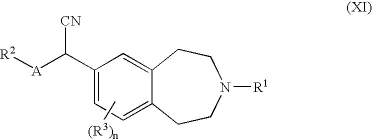
wherein R¹, R³ and n are as defined in claim 25;
(d) deprotecting a compound of formula (I) which is protected; or
(e) interconversion from one compound of formula (I) to another.
